SEQUENCE LISTING (55 sequences)

SEQ ID NO: 1 - protein, 483 aa, synthetic construct
  REGION 1..483: note = Artificial enzyme variant
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGTQRDYLDHPDVIGWTREGDGVHADSGLATLMSDGPGGSKWMEVGKNNAGEVWYDITGNQTNTVTINKDGWGQFHVSGGSVSIYVQQ

SEQ ID NO: 2 - protein, 485 aa, synthetic construct
  REGION 1..485: note = Artificial enzyme variant
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 3 - protein, 485 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 4 - protein, 485 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 5 - protein, 485 aa, synthetic construct
  REGION 1..485: note = Artificial enzyme variant
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 6 - protein, 482 aa, synthetic construct
  REGION 1..482: note = Artificial enzyme variant
HDGTNGTIMQYFEWNVPNDGQHWNRLHNNAQNLKNAGITAIWIPPAWKGTSQSDTGYGAYDLYDLGEFNQRGTVRTKYGTKAELERAIRSLKANGIQVYGDVVMNHKAGADQTEQVQAVEVNPQNRNQEVSGTYQIEAWTGFNFPGRGNQHSSFKWRWYHFDGTDFDQSRGLSNRIYKFRTKAWDWEVDTEFGNYDYLMYADLDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLAARQQYAYGTQHDYLDNQDVIGWTREGDSAHAGSGLATVMSDGPGGSKTMYVGTAHAGQVFKDITGNRTDTVTINSAGNGTFRCNKGSVSIWVKQ

SEQ ID NO: 7 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 8 - protein, 483 aa, Bacillus licheniformis [sequence omitted]

SEQ ID NO: 9 - protein, 583 aa, Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 10 - protein, 485 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 11 - protein, 480 aa, Bacillus sp.
DGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 12 - protein, 485 aa, Bacillus sp.
HHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 13 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 14 - protein, 483 aa, Bacillus subtilis
HHNGTNGTLMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRANTHSNFKWRWYHFDGVDWDQSRKLNNRIYKFRTKAWDWEVDTEFGNYDYLLYADIDMDHPEVVNELRNWGVWYTNTLGLDGFRIDAVKHIKYSFTRDWINHVRSAIGKNMFAVAEFWKNDLGAIENYLNKTNWNHSVFDVPLHFNLYYASKSGGNYDMRQIFNGTVVQKHPTHAVTFVDNHDSQPEESLESFVREWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

SEQ ID NO: 15 - protein, 485 aa, Bacillus sp.
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 16 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 17 - protein, 275 aa, Bacillus amyloliquefaciens [sequence omitted]

SEQ ID NO: 18 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 19 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSFGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGDSFYYGKGLINVQAASN

SEQ ID NO: 20 - protein, 311 aa, Bacillus sphaericus [sequence omitted]

SEQ ID NO: 21 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 22 - protein, 269 aa, synthetic construct
  REGION 1..269: note = Artificial enzyme variant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 23 - protein, 269 aa, synthetic construct
  REGION 1..269: note = Artificial enzyme variant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISAPASYANAMAVGATDQNNNRASFSQYGPGLDIVAPGVNVQSTYPGSTYASLNGTSMATPHVAGAAALVKQKNPSWSNVQIRNHLKNTATSLGSTNLYGSGLVNAEAATR

SEQ ID NO: 24 - protein, 269 aa, synthetic construct
  REGION 1..269: note = Artificial enzyme variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 25 - protein, 311 aa, synthetic construct
  REGION 1..311: note = Artificial enzyme variant
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 26 - protein, 270 aa, synthetic construct
  REGION 1..270: note = Artificial enzyme variant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASEGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 27 - protein, 270 aa, synthetic construct
  REGION 1..270: note = Artificial enzyme variant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGAADGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 28 - protein, 269 aa, synthetic construct
  REGION 1..269: note = Artificial enzyme variant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNDGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNEYGSGLVNAEAATR

SEQ ID NO: 29 - protein, 269 aa, synthetic construct
  REGION 1..269: note = Artificial enzyme variant
AQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGMGSVSSIAQGLEWAGNNVMHVANLSLGLQAPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 30 - protein, 274 aa, synthetic construct
  REGION 1..274: note = Artificial enzyme variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSSGSGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 31 - protein, 289 aa, synthetic construct
  REGION 1..289: note = Artificial enzyme variant
ALTTQSNAPWGLAAISRRTPGGSTYTYDTTAGAGTYGYVVDSGINTAHTDFGGRASLGYNAAGGAHTDTLGHGTHVAGTIASNTYGVAKRANVISVKVFVGNQASTSVILAGFNWAVNDITSKNRASRSVINMSLGGPSSQTWATAINAAYSQGVLSVVAAGNGDSNGRPLPASGQSPANVPNAITVAAADSSWRTASFTNYGPEVDVFGPGVNIQSTWYTSNSATNTISGTSMACPHVAGLALYLQALENLNTPAAVTNRIKSLATTGRITGSLSGSPNAMAFNGATA

SEQ ID NO: 32 - protein, 275 aa, synthetic construct
  REGION 1..275: note = Artificial enzyme variant
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 33 - protein, 275 aa, synthetic construct
  REGION 1..275: note = Artificial enzyme variant
AQSVPYGVSQIKAPALHSQGYTGGNVKVAVIDSGIDSSHPDLKVAGGASMVPGETNPFQDNNSHGTHVAGTVAALNNNIGVLGVAPSASLYAVKVLGADGNGQYSWIINGIEWAIANNMDVINMSLGAPSGSAALKAAVDKAVASGVVVVAAAGNEGTSGSSSTVGYPGKYPSVIAVGAVDSSNQRASFSSVGPELDVMAPGVSIQSTLPGNKYGAQNGTSMASPHVAGAAALILSKHPNWTNTQVRSSLENTTTKLGDSFYYGKGLINVQAAAQ

SEQ ID NO: 34 - protein, 275 aa, synthetic construct
  REGION 1..275: note = Artificial enzyme variant
AQSVPYGVSQIKAPALHSQGYTGGNVKVAVIDTGIDSSHPDLKVAGGASMVPGETNPFQDNNSHGTHVAGTVAALNDNIGVLGVAPSASLYAVKVLGADGNGQYSWIINGIEWAIANNMDVINMSLGAPSGSAALKAAVDKAVASGVVVVAAAGNEGTSGSSSTVGYPGKYPSVIAVGAVDSSNQRASFSSVGPELDVMAPGVSIQSTLPGNKYGAQNGTSMASPHVAGAAALILSKHPNWTNTQVRSSLENTTTKLGDSFYYGKGLINVQAAAQ

SEQ ID NO: 35 - protein, 490 aa, synthetic construct
  REGION 1..490: note = Artificial enzyme variant
MSIIKKVPLIFLCLLMFATSLFIFKPEVKAATGFYVNGNTLYDATGSPFVMRGINHAHSWFKDDSSTAIPAIAKTGANTIRIVLSDGSQYTKDDINTVKSLISLAEKNNLIAILEVHDATGNDAVSSLNDAVSYWISIKEALIGKEDRVLINIANEWYGTWDGASWASGYKQAIPKLRDAGLSHTLIVDSAGWGQYPESIHQYGKDVFNADPLKNTMFSIHMYEYAGGDASTIKSNIDGVLNQDLALIIGEFGHKHTNGDVDEETIMSYSQQKNVGWLAWSWKGNGPEWSYLDLSNDWAGDNLTSWGNTIVNGANGLKATSKISPVFDGGDHPGGSGGTENTLYNFETETQSWSGGNVMAGPWSTNEWASKDNYSLKADVQLNNNSQHYLSLTQNQNFSGKSQLKATVKHADWGNLGNGINAQLYVKTGSDWKWFDGESVEINSSNGTILTLDLSSISDLNDIKEIGVQFMGSSKSSGQTAVYVDNVTIQ

SEQ ID NO: 36 - protein, 460 aa, synthetic construct
  REGION 1..460: note = Artificial enzyme variant
ATGFYVNGNTLYDATGSPFVMRGINHAHSWFKDDSSTAIPAIAKTGANTIRIVLSDGSQYTKDDINTVKSLISLAEKNNLIAILEVHDATGNDAVSSLNDAVSYWISIKEALIGKEDRVLINIANEWYGTWDGASWASGYKQAIPKLRDAGLSHTLIVDSAGWGQYPESIHQYGKDVFNADPLKNTMFSIHMYEYAGGDASTIKSNIDGVLNQDLALIIGEFGHKHTNGDVDEETIMSYSQQKNVGWLAWSWKGNGPEWSYLDLSNDWAGDNLTSWGNTIVNGANGLKATSKISPVFDGGDHPGGSGGTENTLYNFETETQSWSGGNVMAGPWSTNEWASKDNYSLKADVQLNNNSQHYLSLTQNQNFSGKSQLKATVKHADWGNLGNGINAQLYVKTGSDWKWFDGESVEINSSNGTILTLDLSSISDLNDIKEIGVQFMGSSKSSGQTAVYVDNVTIQ

SEQ ID NO: 37 - protein, 297 aa, synthetic construct
  REGION 1..297: note = Artificial enzyme variant
ATGFYVNGNTLYDATGSPFVMRGINHAHSWFKDDSSTAIPAIAKTGANTIRIVLSDGSQYTKDDINTVKSLISLAEKNNLIAILEVHDATGNDAVSSLNDAVSYWISIKEALIGKEDRVLINIANEWYGTWDGASWASGYKQAIPKLRDAGLSHTLIVDSAGWGQYPESIHQYGKDVFNADPLKNTMFSIHMYEYAGGDASTIKSNIDGVLNQDLALIIGEFGHKHTNGDVDEETIMSYSQQKNVGWLAWSWKGNGPEWSYLDLSNDWAGDNLTSWGNTIVNGANGLKATSKISPVF

SEQ ID NO: 38 - protein, 298 aa, synthetic construct
  REGION 1..298: note = Artificial enzyme variant
MATGFYVSGNKLYDSTGKEFVMRGVNHGHTWFKNDLNEAIPAIAKTGANTVRIVLSNGVQYTRDDLDAVKNIINVVNQNKMIAVLEVHDATGKDDYDSLDAAINYWISIKEALIGKEDRVIVNIANEWMGTWNGSAWADGYKKAIPKLRNAGIKNTLIVDAAGWGQYPQSIVDYGQSVFAADSLKNTVFSIHMYEYAGKDAATVKANMENVLNKGLALIIGEFGCYHVNGDVDELAIMRYGQELGVGWLAWSWYGNSDGLRYLDMATGPNGSLTSFGNTVVNDTYGIKNTSQKAGIFQ

SEQ ID NO: 39 - protein, 289 aa, synthetic construct
  REGION 1..289: note = Artificial enzyme variant
QNGFHVSGTELLDKNGDPYVMRGVNHGHSWFKQDLEEAIPAIAETGANTVRMVLSNGQQWEKDDASELARVLAATETYGLTTVLEVHDATGSDDPADLEKAVDYWIEMADVLKGTEDRVIINVANEWYGSWRSDVWAEAYAQAIPRLRSAGLSHTLMVDAAGWGQYPASIHERGADVFASDPLKNTMFSIHMYEYAGADRATIAYNIDRVLAENLAVVIGEFGHRHHDGDVDEDAILAYTAERQVGWLAWSWYGNSGGVEYLDLAEGPSGPLTSWGKRIVYGENGLKSD

SEQ ID NO: 40 - protein, 298 aa, synthetic construct
  REGION 1..298: note = Artificial enzyme variant
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVF

SEQ ID NO: 41 - protein, 309 aa, synthetic construct
  REGION 1..309: note = Artificial enzyme variant
ANSGFYVSGTTLYDANGNPFVMRGINHGHTWYKDQATTAIEGIANTGANTVRIVLSDGGQWTKDDIHTVRNLISLAEDNHLVAVLEVHDATGQDDIASLNRAVDYWIEMRSALIGKEKTVIINIANEWFGSWEGDPWARGYKQAIPRLRNAGLNHTLMVDAAGWGQFPQSIHDYGREVFNADPQRNTMFSIHMYEYAGGTARQVRTNIDGVLNQDLALVIGEFGHRHTNGDVDEATIMSYSEQRGVGWLAWSWKGNGPEFEYLDLSNDWAGNNLTAWGNTIVHGPYGIRETSRPVTVFTGGGSDGGTSP

SEQ ID NO: 42 - protein, 415 aa, synthetic construct
  REGION 1..415: note = Artificial enzyme variant
QKPGETKEVHPQLTTFRCTKRGGCKPATNFIVLDSLSHPIHRAEGLGPGGCGDWGNPPPKDVCPDVESCAKNCIMEGIPDYSQYGVTTNGTSLRLQHILPDGRVPSPRVYLLDKTKRRYEMLHLTGFEFTFDVDATKLPCGMNSALYLSEMHPTGAKSKYNPGGAYYGTGYCDAQCFVTPFINGLGNIEGKGSCCNEMDIWEANSRASHVAPHTCNKKGLYLCEGEECAFEGVCDKNGCGWNNYRVNVTDYYGRGEEFKVNTLKPFTVVTQFLANRRGKLEKIHRFYVQDGKVIESFYTNKEGVPYTNMIDDEFCEATGSRKYMELGATQGMGEALTRGMVLAMSIWWDQGGNMEWLDHGEAGPCAKGEGAPSNIVQVEPFPEVTYTNLRWGEIGSTYQEVQKPKPKPGHGPRSD

SEQ ID NO: 43 - protein, 278 aa, synthetic construct
  REGION 1..278: note = Artificial enzyme variant
ASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNQFDIAMPGGGVGIFNGCSSQFGGLPGAQYGGISSRDQCDSFPAPLKPGCQWRFDWFQNADNPTFTFQQVQCPAEIVARSGCKRNDDSSFPVFTPPSGGNGGTGTPTSTAPGSGQTSPGGGSGCTSQKWAQCGGIGFSGCTTCVSGTTCQKLNDYYSQCL

SEQ ID NO: 44 - protein, 284 aa, synthetic construct
  REGION 1..284: note = Artificial enzyme variant
ADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 45 - protein, 214 aa, synthetic construct
  REGION 1..214: note = Artificial enzyme variant
ANGQSTRYWDCCKPSCGWRGKGPVNQPVYSCDANFQRIHDFDAVSGCEGGPAFSCADHSPWAINDNLSYGFAATALSGQTEESWCCACYALTFTSGPVAGKTMVVQSTSTGGDLGSNHFDLNIPGGGVGLFDGCTPQFGGLPGARYGGISSRQECDSFPEPLKPGCQWRFDWFQNADNPSFTFERVQCPEELVARTGCRRHDDGGFAVFKAPSA

SEQ ID NO: 46 - protein, 773 aa, synthetic construct
  REGION 1..773: note = Artificial enzyme variant
AEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPINDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSADGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEE

SEQ ID NO: 47 - protein, 524 aa, synthetic construct
  REGION 1..524: note = Artificial enzyme variant
VVHGQTAKTITIKVDTFKDRKPISPYIYGTNQDLAGDENMAARRLGGNRMTGYNWENNMSNAGSDWQHSSDNYLCSNGGLTQAECEKPGAVVTSFHDQSLKLGTYSLVTLPMAGYVAADGNGSVQESEAAPSARWNQVVNAKNAPFQLQPDLNDNYVYVDEFVHFLVNKYGTASTKAGVKGYALDNEPALWSHTHPRIHPEKVGAKELVDRSVSLSKAVKAIDAGAEVFGPVLYGFGAYKDLQTAPDWDSVKGNYSWFVDYYLDQMRLSSQVEGKRLLDVFDVHWYPEAMGGGIRITNEVGNDETKKARMQAPRTLWDPTYKEDSWIAQWFSEFLPILPRLKQSVDKYYPGTKLAMTEYSYGGENDISGGIAMTDVLGILGKNDVYMANYWKLKDGVNNYVSAAYKLYRNYDGKNSTFGDTSVSAQTSDIVNSSVHASVTNASDKELHLVVMNKSMDSAFDAQFDLSGAKTYISGKVWGFDKNSSQIKEAAPITQISGNRFTYTVPPLTAYHIVLTTGNDTSPV

SEQ ID NO: 48 - protein, 7 aa, synthetic construct
ADGEGAI

SEQ ID NO: 49 - protein, 7 aa, synthetic construct
ADGDGAI

SEQ ID NO: 50 - protein, 7 aa, synthetic construct
ADGDGSV

SEQ ID NO: 51 - protein, 7 aa, synthetic construct
ADGEGSV

SEQ ID NO: 52 - protein, 8 aa, synthetic construct
AADGSGSV

SEQ ID NO: 53 - protein, 8 aa, synthetic construct
AADGEGSV

SEQ ID NO: 54 - protein, 8 aa, synthetic construct
ASEGEGSV

SEQ ID NO: 55 - protein, 4 aa, synthetic construct
AAPF

(19) United States
(12) Patent Application Publication
(10) Pub. No.: US 2026/0365944 A1
(43) Pub. Date: Nov. 19, 2026

(54) ENZYME COMPOSITIONS COMPRISING AN AMYLASE

(71) Applicant: BASF SE, Ludwigshafen am Rhein (DE)

(72) Inventors: Oliver SPANGENBERG, Ludwigshafen (DE); Sonja KUEBELBECK, Ludwigshafen (DE); Claudia ESPER, Ludwigshafen (DE)

(21) Appl. No.: 19/103,155

(22) PCT Filed: Jul. 31, 2023
(86) PCT No.: PCT/EP2023/071179

§ 371 Date: Feb. 11, 2025

(30) Foreign Application Priority Data
Aug. 11, 2022 (EP) 22189958.6

(51) Int. Cl. C11D 3/386

(52) U.S. Cl. CPC C11D 3/38618; C11D 3/38636; C11D 3/38645; C12Y 302/01004

(57) ABSTRACT

In the present invention new enzyme compositions are provided. More specifically, the present invention provides enzyme compositions comprising an amylase and two or more additional enzymes selected from the group consisting of protease, mannanase, and cellulase and methods of making and using these enzymes compositions.

### FIELD OF THE INVENTION

In the present invention new enzyme compositions are provided. More specifically, the present invention provides enzyme compositions comprising an amylase and two or more additional enzymes selected from the group consisting of protease, mannanase, and cellulase and methods of making and using these enzymes compositions.

### BACKGROUND OF THE INVENTION

Enzymes are increasingly used in various application as sustainable alternative to petrochemistry. Enzymes are biodegradable and can be catalytically active already at lower temperatures, which results in reduction of energy consumption. In particular, in the detergent industry enzymes are implemented in washing formulations to improve cleaning efficiency and to reduce energy consumption in a washing step.

Most typically used enzymes in detergents are proteases and amylases, whereas mannanases and cellulases are used less frequently. Proteases, amylases, mannanases, and cellulases are all hydrolases that differ in the substrate that they can degrade. Proteases degrade proteins, amylases degrade starch, mannanases degrade galactomannan, and cellulases degrade cellulose.

However, successful cleaning often depends on the effectiveness of removing stains comprised of different types of soils. Thus, successful cleaning depends on the effective removal of stains comprising a complex mixture of protein, starch, galactomannan, cellulose, and other types of partially undefined soiling. The inventors of the present application identified combinations of specific enzymes particularly suitable to be used for the removal of such complex stains.

### BRIEF SUMMARY OF THE INVENTION

The present invention is directed to a composition comprising two or more enzymes selected from a different enzyme class (also called “enzyme blend” herein). Specifically, the present invention is directed to a composition comprising an amylase and one or more, preferably two or more enzymes selected from the group consisting of protease, mannanase, and cellulase. The present invention is also directed to a method of making the enzyme composition.

Using the composition of specific enzymes of the present invention provides a beneficial cleaning effect when cleaning an object. Thus, the present invention is also directed to the use of the enzyme composition for cleaning an object and to a detergent composition comprising the enzyme composition.

### DETAILED DESCRIPTION OF THE INVENTION

The present invention may be understood more readily by reference to the following detailed description of the embodiments of the invention and the examples included herein.

Although the present invention will be described with respect to particular embodiments, this description is not to be construed in a limiting sense.

### DEFINITIONS

Unless otherwise noted, the terms used herein are to be understood according to conventional usage by those of ordinary skill in the relevant art.

Before describing in detail exemplary embodiments of the present invention, definitions important for understanding the present invention are given. Unless stated otherwise or apparent from the nature of the definition, the definitions apply to all compounds, methods and uses described herein.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all of these publications and those references cited within those publications in their entireties are hereby incorporated by reference into this application in order to more fully describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of members, this is meant to also encompass a group which consists of these members only.

“Variant” enzymes differ from “parent” enzymes by certain amino acid alternations, preferably amino acid substitutions at one or more amino acid positions.

In describing the variants of the present invention, the abbreviations for single amino acids are used according to the accepted IUPAC single letter or three letter amino acid abbreviation. “Amino acid alteration” as used herein refers to amino acid substitution, deletion, or insertion. “Substitutions” are described by providing the original amino acid followed by the number of the position within the amino acid sequence, followed by the amino acid, which substitutes the original amino acid. For example, the substitution of histidine at position 120 with alanine is designated as “His120Ala” or “H120A”. Substitutions can also be described by merely naming the resulting amino acid in the variant without specifying the amino acid of the parent at this position, e.g., “X120A” or “120A” or “Xaa120Ala” or “120Ala”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by, e.g., “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as, e.g., a Lys and an Ala after Gly180 this may be indicated as: “Gly180GlyLysAla” or “G195GKA”.

In cases where a substitution and an insertion occur at the same position, this may be indicated as “S99SD+S99A” or in short “S99AD”. Variants comprising multiple alterations are separated by “+”, e.g., “Arg170Tyr+Gly195Glu”, “R170Y+G195E” or “X170Y+X195E” representing a substitution of arginine and glycine at positions 170 and 195 with tyrosine and glutamic acid, respectively. Alternatively, multiple alterations may be separated by space or a comma, e.g., “R170Y G195E” or “R170Y, G195E” respectively. Where different alternative alterations can be introduced at a position, the different alterations are separated by a comma, e.g., “Arg170Tyr, Glu” and “R170T, E”, respectively, represents a substitution of arginine at position 170 with tyrosine or glutamic acid. Alternative substitutions at a particular position can also be indicated as “X120A,G,H”, “120A,G,H”, “X120A/G/H”, or “120A/G/H”. Alternatively, different alterations or optional substitutions may be indicated in brackets, e.g., “Arg170[Tyr, Gly]” or “Arg170{Tyr, Gly}” or in short “R170 [Y, G]” or “R170 {Y, G}”.

The term “native” (or naturally or wildtype or endogenous) cell or organism or polynucleotide or polypeptide refers to the cell or organism or polynucleotide or polypeptide as found in nature (i.e., without there being any human intervention).

The term “introduction of at least two negative charges” into a particular amino acid sequence refers to the increase of the net charge of the particular amino acid sequence by at least two negative charges. Such increase of the net charge of the particular amino acid sequence by at least two negative charges is achieved by altering the amino acid sequence and can be reached by one or more amino acid sequence alterations selected from the group consisting of substitution, deletion and insertion, preferably by one or more amino acid substitutions. The increase of the net charge of the particular amino acid sequence by at least two negative charges can be achieved by removing positive charges or by introducing negative charges or by combinations thereof. The four amino acids aspartic acid (Asp, D), glutamic acid (Glu, E), lysine (Lys, K), and arginine (Arg, R) have a side chain which can be charged at neutral pH. At pH 7.0, two are negatively charged: aspartic acid (Asp, D) and glutamic acid (Glu, E) (acidic side chains), and two are positively charged: lysine (Lys, K) and arginine (Arg, R) (basic side chains). Thus, the introduction of at least two negative charges in the amino acid sequence can be reached for instance by substituting arginine by glutamic acid, substituting two non-charged leucine residues by two glutamic acid residues, by inserting two aspartic acid residues or by deleting two lysine residues. The introduction of at least two negative charges by modification of the amino acid sequence is evaluated preferably under conditions usually occurring in a washing step, preferably at pH 6-11, preferably at pH 7-9, more preferably at pH 7.5-8.5, further preferred at pH 7.0-8.0, most preferably at pH 7.0 or pH 8.0.

The term “heterologous” (or exogenous or foreign or recombinant or non-native or non-naturally) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, e.g., a stronger promoter. Similarly, the term “heterologous” (or exogenous or foreign or recombinant or non-native or non-naturally) polynucleotide refers to a polynucleotide that is not native to the host cell, a polynucleotide native to the host cell in which structural modifications, e.g., deletions, substitutions, and/or insertions, have been made by recombinant DNA techniques to alter the native polynucleotide, or a polynucleotide native to the host cell whose expression is quantitatively altered as a result of manipulation of the regulatory elements of the polynucleotide by recombinant DNA techniques, e.g., a stronger promoter, or a polynucleotide native to the host cell, but integrated not within its natural genetic environment as a result of genetic manipulation by recombinant DNA techniques. With respect to the relation between two or more polynucleotide sequences or the relation between two or more amino acid sequences, the term “heterologous” is used to characterize that the two or more polynucleotide sequences or two or more amino acid sequences are naturally not occurring in the specific combination with each other.

For the purpose of the invention, “recombinant” (or transgenic) with regards to a cell or an organism means that the cell or organism contains a heterologous polynucleotide, which is introduced by man using gene technology. With regards to a polynucleotide “recombinant” includes all constructs produced by using gene technology/recombinant DNA techniques in which either
(a) the sequence of the polynucleotide or a part thereof, or
(b) one or more genetic control sequences, which are operably linked to the polynucleotide, including but not limited to a promoter, or
(c) both a) and b)

are not located in their wildtype genetic environment or have been modified by man.

A “synthetic” compound is obtained by in vitro chemical and/or enzymatic synthesis.

Variant polynucleotide and variant polypeptide sequences may be defined by their sequence identity when compared to another sequence. Sequence identity usually is provided as “% sequence identity” or “% identity”. For calculation of sequence identities, in a first step a sequence alignment is produced. According to this invention, a pairwise global alignment is produced, meaning that two sequences are aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62). After aligning two sequences, in a second step, an identity value is determined from the alignment produced. For this purpose, the %-identity is calculated by dividing the number of identical residues by the length of the alignment region which is showing the respective sequence of the present invention over its complete length multiplied with 100: %-identity=(identical residues/length of the alignment region which is showing the respective sequence of the present invention over its complete length)*100.

For calculating the percent identity of two nucleic acid sequences the same applies as for the calculation of percent identity of two amino acid sequences with some specifications. For nucleic acid sequences encoding for a protein the pairwise alignment shall be made over the complete length of the coding region of the sequence of this invention from start to stop codon excluding introns. Introns present in the other sequence, to which the sequence of this invention is compared, shall also be removed for the pairwise alignment. After aligning two sequences, in a second step, an identity value is determined from the alignment produced. Percent identity is calculated by %-identity=(identical residues/length of the alignment region which is showing the sequence of the invention from start to stop codon excluding introns over its complete length)*100.

Moreover, the preferred alignment program for nucleic acid sequences implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Variant polypeptides may also be defined by their sequence similarity when compared to another sequence. Sequence similarity usually is provided as “% sequence similarity” or “%-similarity”. % sequence similarity takes into account that defined sets of amino acids share similar properties, e.g. by their size, by their hydrophobicity, by their charge, or by other characteristics. Herein, the exchange of one amino acid with a similar amino acid may be called “conservative mutation”.

Similar amino acids according to the invention are defined as follows, which shall also apply for determination of %-similarity according to this invention, which is also in accordance with the BLOSUM62 matrix as for example used by program “NEEDLE”, which is one of the most used amino acids similarity matrix for database searching and sequence alignments:
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W

For calculation of sequence similarity, in a first step a sequence alignment is produced as described above. After aligning two sequences, in a second step, a similarity value is determined from the alignment produced. For this purpose, the %-similarity is calculated by dividing the number of identical residues plus the number of similar residues by the length of the alignment region which is showing the sequence of the invention over its complete length multiplied with 100: %-similarity=[(identical residues+similar residues)/length of the alignment region which is showing the sequence of the invention over its complete length]*100.

A “fragment” or “subsequence” as used herein is a portion of a polynucleotide or an amino acid sequence. The term “functional fragment” refers to any nucleic acid or amino acid sequence which comprises merely a part of the full-length amino acid sequence, respectively, but still has the same or similar activity and/or function. Preferably, the functional fragment is at least 75%, at least 76%, at least 77%, at least 78%, at least 79%, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, or at least 99.5% identical to the original full length amino acid sequence. The functional fragment comprises consecutive nucleotides or amino acids compared to the original nucleic acid or original amino acid sequence, respectively.

“Genetic construct” or “expression cassette” as used herein, is a nucleic acid molecule composed of at least one sequence of interest to be expressed, operably linked to one or more control sequences (at least to a promoter) as described herein.

The term “vector” as used herein comprises any kind of construct suitable to carry foreign polynucleotide sequences for transfer to another cell, or for stable or transient expression within a given cell. The term “vector” as used herein encompasses any kind of cloning vehicles, such as but not limited to plasmids, phagemids, viral vectors (e.g., phages), bacteriophage, baculoviruses, cosmids, fosmids, artificial chromosomes, and any other vectors specific for specific hosts of interest. Foreign polynucleotide sequences usually comprise a coding sequence which may be referred to herein as “gene of interest”. The gene of interest may comprise introns and exons, depending on the kind of origin or destination of host cell.

The term “introduction of a polynucleotide” or “transformation of a polynucleotide” as referred to herein encompasses the transfer of an exogenous polynucleotide into a host cell, irrespective of the method used for transfer. That is, the term “transformation of a polynucleotide” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, NY), but it encompasses any further kind polynucleotide transfer methods such as, but not limited to, transduction or transfection.

A polynucleotide encoding a polypeptide may be “expressed”. The term “expression” or “gene expression” means the transcription of a gene or genes or genetic construct into structural RNA (e.g., rRNA, tRNA) or mRNA with or without subsequent translation of the latter into a protein. The process includes transcription of DNA and processing of the resulting mRNA product.

The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively. The resulting solution after purifying the enzyme of interest from the fermentation broth is called herein “purified enzyme solution”.

“Protein formulation” (or “enzyme preparation”), e.g., “protein variant formulation”, means any non-complex formulation comprising a small number of ingredients, wherein the ingredients serve the purpose of stabilizing the proteins comprised in the protein formulation and/or the stabilization of the protein formulation itself. Preferably, the non-complex protein formulation comprises the protein in higher concentrations than the complex formulation, e.g., than a detergent composition. Thus, preferably the non-complex protein formulation is a concentrated protein variant formulation. Preferably, non-complex protein formulations comprise 2 to 120 mg/g active enzyme, wherein complex formulations, like detergent compositions, comprise 0.002 to 6 mg/g active enzyme.

“Enzyme properties” include, but are not limited to catalytic activity, substrate/cofactor specificity, product specificity, stability in the course of time, thermostability, pH stability, and chemical stability. “Enzymatic activity” or “catalytic activity” means the catalytic effect exerted by an enzyme, expressed as units per milligram of enzyme (specific activity) or molecules of substrate transformed per minute per molecule of enzyme (molecular activity). Enzymatic activity can be specified by the enzymes actual function, e.g., proteases exerting proteolytic activity by catalyzing hydrolytic cleavage of peptide bonds, lipases exerting lipolytic activity by hydrolytic cleavage of ester bonds, amylases activity involves hydrolysis of glycosidic linkages in polysaccharides, etc.

The term “enzyme stability” according to the current invention relates to the retention of enzymatic activity as a function of time during storage or operation. Retention of enzymatic activity as a function of time during storage is called “storage stability” and is preferred within the context of the invention.

To determine and quantify changes in catalytic activity of enzymes stored or used under certain conditions over time, the “initial enzymatic activity” is measured under defined conditions at time zero (100%) and at a certain point in time later (x %). By comparison of the values measured, a potential loss of enzymatic activity can be determined in its extent. The extent of enzymatic activity loss determines an enzyme's stability or non-stability as such or in a formulation.

“Enzyme inhibitors” as used herein are compounds that slow down or halt enzymatic activity. Enzyme inhibitors frequently also stabilize the enzyme in its three-dimensional structure. Hence, enzyme inhibitors usually also act as “enzyme stabilizers”.

“pH stability” refers to the ability of an enzyme to exert enzymatic activity after exposure to certain pH value.

The terms “thermal stability”, “thermostability” or “temperature-dependent activity” refer to the ability of an enzyme to exert catalytic activity or wash performance after exposure to elevated temperatures, preferably at a temperature of 40 °C for 28 days, more preferably 56 days, preferably in a detergent composition (preferably, in model ES1-C detergent), or at 92 °C for at least 10 min.

The terms “detergent stability” or “stability under storage in a detergent composition” refer to the ability of an enzyme to exert catalytic activity or wash performance after storage in a detergent composition, preferably at a temperature of 40 °C or 50 °C for 28 days, more preferably 56 days, in a detergent composition (preferably, in model ES1-C detergent).

As used herein, “wash performance” (also called herein “cleaning performance”) of an enzyme refers to the contribution of the enzyme to the cleaning performance of a detergent composition, i.e. the cleaning performance added to the detergent composition by the performance of the enzyme. The term “wash performance” is used herein similarly for laundry and hard surface cleaning. Wash performance is compared under relevant washing conditions. The term “relevant washing conditions” is used herein to indicate the conditions, particularly washing temperature, time, washing mechanics, concentration of detergent components in the wash liquor (i.e., sud concentration), type of detergent and water hardness, actually used in households in a detergent market segment. The term “improved wash performance” is used to indicate that a better end result is obtained in stain removal under relevant washing conditions, or that less enzyme, on weight basis, is needed to obtain the same end result relative to the corresponding control conditions.

As used herein, the term “specific performance” refers to the cleaning and removal of specific stains or soils per unit of active enzyme. In some embodiments, the specific performance is determined using stains or soils such as egg, egg yolk, milk, grass, minced meat blood, chocolate sauce, baby food, sebum, etc.

“Detergent composition”, “detergent formulation”, or “detergent” means compositions designated for cleaning soiled material. Detergent compositions according to the invention include detergent compositions for different applications such as laundry and hard surface cleaning. The term “detergent component” is defined herein to mean a type of chemical, which can be used in detergent compositions. A typical detergent component is a surfactant. “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric. The term “effective amount of a detergent component” includes amounts of certain components to provide effective stain removal and/or effective cleaning conditions (e.g. pH, temperature, water hardness), amounts of certain components to effectively provide optical benefits (e.g. optical brightening, dye transfer inhibition, color care), and amounts of certain components to effectively aid the processing (maintain physical characteristics during processing, storage and use; e.g. rheology modifiers, hydrotropes, desiccants). The term “laundry” or “laundering” relates to both household laundering and industrial laundering and means the process of treating textiles and/or fabrics with a solution containing a detergent composition of the present invention. The laundering process may be carried out by using technical devices such as a household or an industrial washing machine. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics made of these materials such as garments, cloths and other articles. The terms “fabric” (a textile made by weaving, knitting or felting fibers) or “garment” (any article of clothing made of textile) as used herein, mean to include the broader term textile as well.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheep wool, angora, mohair, cashmere). Examples for fibers of synthetic origin are polyurethane fibers such as Spandex® or Lycra®, polyester fibers, polyolefins such as elastofin, or polyamide fibers such as nylon. Fibers may be single fibers or parts of textiles such as knitwear, woven or non-woven fabrics.

The term “hard surface cleaning” relates to both household hard surface cleaning and industrial hard surface cleaning and means the process of treating hard surfaces with a solution containing a detergent composition of the present invention. Hard surfaces may include any hard surfaces in the household or industry, such as floors, furnishing, walls, sanitary ceramics, glass, metallic surfaces including medical devices, cutlery, and dishes. A particular form of hard surface cleaning is dishwashing, manual dish washing (MDW) or automatic dishwashing (ADW). The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics. Cleaning performance is evaluated under relevant cleaning conditions. The term “relevant cleaning conditions” herein refers to the conditions, particularly cleaning temperature, time, cleaning mechanics, suds concentration, type of detergent and water hardness, actually used in laundry machines, automatic dish washers or in manual cleaning processes.

The term “medical device cleaning” refers to the cleaning step in reprocessing reusable medical devices. Medical device cleaning methods can be divided into two categories, manual and mechanical/automated cleaning methods. Manual cleaning is used when mechanical units are not available or medical devices to be cleaned are too fragile or difficult to clean with a mechanical unit. Mechanical/automated cleaning methods remove soiling and microorganisms through an automated cleaning and rinsing process, this includes ultrasonic cleaning and washing.

In the field of detergency, usually the term “stains” is used with reference to laundry, e.g., cleaning for textiles, fabric, or fibers, whereas the term “soils” is usually used with reference to hard surface cleaning, e.g., cleaning of dishes and cutlery. However, herein the terms “stain” and “soil” are used interchangeably.

A “sequestering builder” as used herein is different from a precipitating builder in that no significant amount of precipitate is formed when the builder is used in an amount sufficient to combine with all of the calcium ions in an aqueous solution with 7° dH hardness (German hardness) initially at neutral pH. A “strong builder” is classified as high efficiency chelator that can bind divalent cations such as Ca2+ strongly with a logarithmic stability constant (Log K_{Ca}) of the cation/chelator complex of above 4, particular above 5, above 6 or above 7. The stability constants are determined at an ionic strength of 0.1 M and at a temperature of 25 °C. A “strong sequestering builder” combines both of the above-mentioned properties.

A “complex stain” (also called “mixed stain” herein) is a stain comprising more than one soil component selected from fat, protein, starch, mannan, cellulose, preferably wherein the stain comprises a thickening agent like galactomannan or locust bean gum. An example of a complex stain is a salad dressing stain. Further food stains, like chocolate pudding or baby food, are considered to be complex stains.

The term “synergy” or “synergistic effect” as used herein refers to the interaction of two or more enzymes to produce a combined effect, e.g., wash performance, greater than the sum of their separate effects.

The term “low temperature” as used herein refers to a temperature range equal or below 40 °C, preferably 10-40 °C, more preferably 20-40 °C, more preferably 20-35 °C.

### DETAILED DESCRIPTION

In the present invention new enzyme compositions are provided. More specifically, the present invention provides enzyme compositions comprising an amylase and two or more additional enzymes selected from the group consisting of protease, mannanase, and cellulase and methods of making and using these enzymes compositions.

Particularly, the present inventors found that using in such enzyme compositions an amylase, wherein the amylase is an amylase variant of a parent amylase, wherein said amylase variant comprises
(i) an amino acid substitution at position 25 according to the numbering of SEQ ID NO: 2,
(ii) an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2, and
(iii) at least 60%, preferably at least 91% identity, but less than 100% sequence identity with any of SEQ ID NO: 1-15, preferably SEQ ID NO: 1,

provides a beneficial cleaning effect.

### Compositions

In one embodiment, the present invention is directed to an enzyme composition.

In particular, the present invention is directed to a composition comprising an amylase and one or more, preferably two or more enzymes selected from the group consisting of protease, mannanase, and cellulase, wherein
a. the amylase is an amylase variant of a parent amylase, wherein said amylase variant comprises
   (i) an amino acid substitution at position 25 according to the numbering of SEQ ID NO: 2,
   (ii) an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2, and
   (iii) at least 60%, preferably at least 91% identity, but less than 100% sequence identity with any of SEQ ID NO: 1-15, preferably SEQ ID NO: 1,
b. the protease is selected from
   I. a protease variant of a parent protease, wherein the protease variant comprises
      (i) an amino acid sequence comprising compared to SEQ ID NO: 16 at least two additional negative charges in the loop region of residues 98 to 104 according to the numbering of SEQ ID NO: 17, and
      (ii) an amino acid sequence which is at least 60%, but less than 100% identical to SEQ ID NO: 16-34, preferably SEQ ID NO: 16, or
   II. a protease variant of a parent protease, wherein the protease variant comprises
      (i) an amino acid substitution at one or more amino acid positions corresponding to amino acid positions selected from the group consisting of 3, 4, 9, 19, 22, 43, 62, 63, 76, 87, 99, 101, 103, 104, 116, 118, 128, 129, 130, 156, 160, 167, 170, 182, 185, 188, 194, 205, 213, 217, 218, 232, 245, 256, 259, 262, and 271 according to the numbering of SEQ ID NO: 17 and an amino acid sequence which has at least 60%, but less than 100% sequence identity with SEQ ID NO: 22, or
      (ii) an amino acid substitution at one or more amino acid positions corresponding to amino acid positions selected from the group consisting of 24, 53, 76, 78, 101, 128, and 217 according to the numbering of SEQ ID NO: 17 and an amino acid sequence which has at least 60%, but less than 100% sequence identity with SEQ ID NO: 17, or
      (iii) an amino acid substitution at one or more amino acid positions corresponding to amino acid positions selected from the group consisting of 9, 89, 130, 133, 144, 159, 183, 189. 217, 224, 238, 243, 252, 259, 270 and 271 according to the numbering of SEQ ID NO: 19 and an amino acid sequence which has at least 60%, but less than 100% sequence identity with SEQ ID NO: 19, or
      (iv) an amino acid sequence which has at least 60% sequence identity with SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31,
c. the mannanase is selected from
   I. a mannanase variant of a parent mannanase, wherein the mannanase variant comprises
      (i) one or more amino acid substitution selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and
      (ii) an amino acid sequence which is at least 60%, but less than 100% identical to a sequence according to positions 31-490 of SEQ ID NO: 35, or
   II. a mannanase variant of a parent mannanase, wherein the mannanase variant comprises
      (i) an amino acid substitution at one or more amino acid positions corresponding to amino acid positions selected from the group consisting of 24, 123, and 256 according to the numbering of SEQ ID NO: 39 and an amino acid sequence which is at least 60%, but less than 100% identical to SEQ ID NO: 39, or
      (ii) an amino acid substitution at one or more amino acid positions corresponding to amino acid positions selected from the group consisting of 10, 19, 30, 38, 59, 60, 67, 97, 103, 129, 143, 167, 168, 184, 225, 228, 235, 244, 258, 261, and 298 according to the numbering of SEQ ID NO: 38 and an amino acid sequence which is at least 60%, but less than 100% identical to SEQ ID NO: 38, or
      (iii) an amino acid sequence which is at least 60% identical to SEQ ID NO: 40, or
      (iv) an amino acid substitution at one or more amino acid positions corresponding to amino acid positions selected from the group consisting of 30, 81, 93, 95, 118, 138, 139, 200, 202, 210, 260, 283, 288, 294, and 295 according to the numbering of SEQ ID NO: 40 and an amino acid sequence which is at least 60%, but less than 100% identical to SEQ ID NO: 40,
d. the cellulase is selected from
   I. a cellulase variant comprising an amino acid sequence which is at least 60% identical to SEQ ID NO: 42, or
   II. a cellulase variant comprising an amino acid sequence which is at least 60% identical to SEQ ID NO: 46, SEQ ID NO: 43, SEQ ID NO: 45, SEQ ID NO: 44, or SEQ ID NO: 47.

In a preferred embodiment, the present invention is directed to a composition comprising an amylase and one or more, preferably two or more enzymes selected from the group consisting of protease, mannanase, and cellulase, wherein
a. the amylase is selected from an amylase variant of a parent amylase, wherein said amylase variant comprises
   (i) an amino acid substitution at position 25 according to the numbering of SEQ ID NO: 2,
   (ii) an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2,
   (iii) at least 60%, preferably at least 91% identity, but less than 100% sequence identity with any of SEQ ID NO: 1-15, preferably SEQ ID NO: 1,
b. the protease is selected from a protease variant of a parent protease, wherein the protease variant comprises
   (i) an amino acid sequence comprising compared to SEQ ID NO: 16 at least two additional negative charges in the loop region of residues 98 to 104 according to the numbering of SEQ ID NO: 17, and
   (ii) an amino acid sequence which is at least 60%, but less than 100% identical to SEQ ID NO: 16-34, preferably SEQ ID NO: 16,
c. the mannanase is selected from a mannanase variant of a parent mannanase, wherein the mannanase variant comprises
   (i) one or more amino acid substitution selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and
   (ii) an amino acid sequence which is at least 60%, but less than 100% identical to a sequence according to positions 31-490 of SEQ ID NO: 35, and
d. the cellulase is selected from a cellulase variant comprising an amino acid sequence which is at least 60% identical to SEQ ID NO: 42.

In one embodiment, the composition comprises an amylase and a protease, an amylase and a mannanase, or an amylase and a cellulase as described herein. In a preferred embodiment, the composition comprises an amylase and a protease or an amylase and a mannanase as described herein, preferably an amylase and a mannanase as described herein.

In one embodiment, the composition comprises an amylase and a protease and a mannanase as described herein. In one embodiment, the composition comprises an amylase and a protease and a cellulase as described herein. In one embodiment, the composition comprises an amylase and a mannanase and a cellulase as described herein. Preferably, the composition comprises an amylase and a protease and a mannanase as described herein.

In a particularly preferred embodiment, the composition comprises an amylase and a protease, a mannanase, and a cellulase as described herein.

In one embodiment, the composition comprises an amylase and one or more, preferably two or more enzymes selected from the group consisting of protease, mannanase, and cellulase, wherein the composition comprises two or more different amylases, proteases, mannanases, and/or cellulases as described herein, e.g., the combination of an anti-greying and an anti-pilling cellulase.

### Amylases

The enzyme composition of the present invention comprises an amylase.

Amylases and amylase variants described herein have amylase activity (also called herein “amylolytic activity”). “Amylolytic activity” or “amylase activity” describes the capability for the hydrolysis of glucosidic linkages in polysaccharides. Amylase activity may be determined by assays for measurement of amylase activity which are known to those skilled in the art. Examples for assays measuring amylase activity are the Phadebas assay or the EPS assay (“Infinity reagent”). In the Phadebas assay amylase activity is determined by employing Phadebas tablets as substrate (Phadebas Amylase Test, supplied by Magle Life Science). Starch is hydrolyzed by the amylase giving soluble blue fragments. The absorbance of the resulting blue solution, measured spectrophotometrically at 620 nm, is a function of the amylase activity. The measured absorbance is directly proportional to the specific activity (activity/mg of pure amylase protein) of the amylase in question under the given set of conditions.

Alternatively, amylase activity can also be determined by a method employing the Ethyliden-4-nitrophenyl-alpha-D-maltoheptaosid (EPS). D-maltoheptaoside is a blocked oligosaccharide which can be cleaved by an endo-amylase. Following the cleavage, the alpha-glucosidase included in the kit digests the substrate to liberate a free PNP molecule, which has a yellow color and thus can be measured by visible spectophotometry at 405 nm. Kits containing EPS substrate and alpha-glucosidase are manufactured for example by Roche Costum Biotech (cat. No. 10880078103). The slope of the time dependent absorption-curve is directly proportional to the specific activity (activity per mg enzyme) of the amylase in question under the given set of conditions.

The amylase of the enzyme composition is an amylase variant of a parent amylase, wherein said amylase variant comprises
(i) an amino acid substitution at position 25 according to the numbering of SEQ ID NO: 2,
(ii) an amino acid substitution at one or more, preferably two or more, amino acid positions corresponding to amino acid positions selected from the group consisting of 4, 116, 176, 181, 186, 195, 206, 225, 251, 320, 405, and 482 according to the numbering of SEQ ID NO: 2, and
(iii) at least 60%, preferably at least 91% identity, but less than 100% sequence identity with any of SEQ ID NO: 1-15, preferably SEQ ID NO: 1, preferably wherein the parent amylase of the amylase variant is an amylase according to SEQ ID NO: 1 or SEQ ID NO: 2, preferably SEQ ID NO: 1.

The amylase variant is a non-naturally occurring amylase. Preferably, the amylase variant is a purified, isolated, synthetic, and/or recombinant amylase variant. Preferably, the amylase variant is a purified and recombinant amylase variant.

Preferably, the parent amylase for the amylase variant is an amylase having at least 60% sequence identity to any of SEQ ID NO: 1-15, preferably the parent amylase for the amylase variant is an amylase according to any of SEQ ID NO: 1-15. Most preferably the parent amylase for the amylase variant is an amylase according to SEQ ID NO: 1 or SEQ ID NO: 2, preferably SEQ ID NO: 1.

The present invention is directed to an amylase variant of a parent amylase having amylase activity, wherein said variant comprises compared to the parent amylase amino acid alteration, preferably insertion, deletion, substitution, or combination thereof, most preferably substitution, at two or more positions corresponding to positions selected from the group consisting of 4, 25, 116, 176, 181, 186, 195, 206, 225, 251, 320, 405, and 482 according to the numbering of the amino acid sequence set forth in SEQ ID NO: 2.

In an alternative way, one can describe the amino acid positions 4, 25, 116, 176, 181, 186, 195, 206, 225, 251, 320, 405, and 482 (according to the numbering of SEQ ID NO: 2) with reference to the numbering of SEQ ID NO: 1, i.e., according to the numbering of SEQ ID NO: 1. In this alternative way of describing the amino acid positions of the present invention, amino acid positions 4, 25, 176, 186, 251, 405, and 482 according to the numbering of SEQ ID NO: 2 correspond to amino acid positions 4, 25, 116, 176, 181, 184, 193, 204, 223, 249, 318, 403, and 480 according to the numbering of SEQ ID NO: 1.

Preferably, the parent amylase for the amylase variant is an amylase having at least 60% sequence identity with SEQ ID NO: 1 or SEQ ID NO: 2, or any of SEQ ID NO: 3-15, preferably the parent amylase for the amylase variant is an amylase according to SEQ ID NO: 1 or SEQ ID NO: 2, or any of SEQ ID NO: 3-15. Most preferably the parent amylase for the amylase variant is an amylase according to SEQ ID NO: 1.

Preferably, the present invention is directed to an amylase variant comprising one or more amino acid substitutions selected from the group consisting of X4Q, X25H, X116K, X176K, X186E, X195F, X206Y, X225A, X251E, X320K, X405M, and X482W according to the numbering of the amino acid sequence set forth in SEQ ID NO: 2.

Preferably, the present invention is directed to an amylase variant comprising compared to a parent sequence one or more amino acid substitutions selected from the group consisting of X4Q, X25H, X116K, X176K, X186E, X195F, X206Y, X225A, X251E, X320K, X405M, and X482W according to the numbering of the amino acid sequence set forth in SEQ ID NO: 2 and wherein said variant has amylase activity, preferably wherein the parent amylase for the amylase variant is an amylase according to SEQ ID NO: 1 or any amylase having at least 60% sequence identity with SEQ ID NO: 1, most preferably the parent amylase for the amylase variant is an amylase according to SEQ ID NO: 1.

Preferably, in this embodiment, the amino acid residue in the parent amylase at the above cited positions (i.e., X) corresponds to the amino acid residue shown in SEQ ID NO: 1 at the respective position according to the numbering of SEQ ID NO: 2.

Thus, preferably, the present invention is directed to an amylase variant comprising an amino acid substitution at one or more positions corresponding to positions selected from the group consisting of G4, N25, W116, R176, R181, G186, N195, 1206, T225, T251, R320, L405, and Y482 according to the numbering of the amino acid sequence set forth in SEQ ID NO: 2.

In a preferred embodiment, the present invention is directed to an amylase variant comprising an amino acid substitution at one or more positions corresponding to positions selected from the group consisting of G4Q, N25H, W116K, R176K, R181T, G186E, N195F, 1206Y, T225A, T251E, R320K, L405M, and Y482W according to the numbering of the amino acid sequence set forth in SEQ ID NO: 2.

The present invention is preferably directed to an amylase variant comprising an amino acid substitution at position 25 and at one or more positions corresponding to positions selected from the group consisting of 4, 116, 176, 181, 186, 195, 206, 225, 251, 320, 405, and 482 according to the numbering of SEQ ID NO: 2.

Preferably, the present invention is directed to an amylase variant comprising the amino acid substitution X25H and one or more amino acid substitutions selected from the group consisting of X4Q, X116K, X176K, X186E, X195F, X206Y, X225A, X251E, X320K, X405M, and X482W according to the numbering of the amino acid sequence set forth in SEQ ID NO: 2.

Thus, preferably, the present invention is directed to an amylase variant comprising an amino acid substitution at the amino acid position N25 and at one or more positions corresponding to positions selected from the group consisting of G4, W116, R176, R181, G186, N195, 1206, T225, T251, R320, L405, and Y482 according to the numbering of SEQ ID NO: 2.

In a preferred embodiment, the present invention is directed to an amylase variant comprising the amino acid substitution N25H and amino acid substitutions at one or more positions corresponding to positions selected from the group consisting of G4Q, W116K, R176K, R181T, G186E, N195F, 1206Y, T225A, T251E, R320K, L405M, and Y482W according to the numbering of SEQ ID NO: 2.

Preferably, the amylase variant comprises compared to the parent amylase an amino acid substitution at one or more of the amino acid positions (according to the numbering of the amino acid sequence set forth in SEQ ID NO: 2) described below. Preferably, the parent amylase for the amylase variant is an amylase according to any of SEQ ID NO: 1-15, most preferably the parent amylase for the amylase variant is an amylase according to SEQ ID NO: 1. Preferably, the amino acid residue of the parent amylase at the cited positions (i.e., X) corresponds to the amino acid residue shown in SEQ ID NO: 2 at the respective position (according to the numbering of SEQ ID NO: 2).

The amylase variant preferably comprises an amino acid substitution at position 25 (according to the numbering of SEQ ID NO: 2), preferably the substitution X25H. Alternatively, the acid substitution at position 25 is X25Y or X25D.

Preferably, the amylase variant comprises an amino acid substitution at position 4 (according to the numbering of SEQ ID NO: 2), preferably the substitution X4Q.

Preferably, the amylase variant comprises an amino acid substitution at position 116 (according to the numbering of SEQ ID NO: 2), preferably the substitution X116K.

Preferably, the amylase variant comprises an amino acid substitution at position 176 (according to the numbering of SEQ ID NO: 2), preferably the substitution X176K.

Preferably, the amylase variant comprises an amino acid substitution at position 181 (according to the numbering of SEQ ID NO: 2), preferably the substitution X181T.

Preferably, the amylase variant comprises an amino acid substitution at position 186 (according to the numbering of SEQ ID NO: 2), preferably the substitution X186E.

Preferably, the amylase variant comprises an amino acid substitution at position 195 (according to the numbering of SEQ ID NO: 2), preferably the substitution X195F.

Preferably, the amylase variant comprises an amino acid substitution at position 206 (according to the numbering of SEQ ID NO: 2), preferably the substitution X206Y.

Preferably, the amylase variant comprises an amino acid substitution at position 225 (according to the numbering of SEQ ID NO: 2), preferably the substitution X225A.

Preferably, the amylase variant comprises an amino acid substitution at position 251 (according to the numbering of SEQ ID NO: 2), preferably the substitution X251E.

Preferably, the amylase variant comprises an amino acid substitution at position 320 (according to the numbering of SEQ ID NO: 2), preferably the substitution X320K.

Preferably, the amylase variant comprises an amino acid substitution at position 405 (according to the numbering of SEQ ID NO: 2), preferably the substitution X405M.

Preferably, the amylase variant comprises an amino acid substitution at position 482 (according to the numbering of SEQ ID NO: 2), preferably the substitution X482W.

Preferably, the amylase variant additionally comprises a deletion at one or more, preferably at two or more, amino acids corresponding to positions selected from the group consisting of 181, 182, 183 and 184. Preferably, the amylase variant of the present invention having one or more amino acid substitutions as described herein comprises a deletion of one or more amino acids corresponding to positions 183 and 184, preferably a deletion of both amino acids corresponding to positions 183 and 184 (according to the numbering of SEQ ID NO: 2). Preferably, the amylase variant of the present invention having one or more amino acid substitutions as described herein comprises a deletion of one or more, preferably of two or more, most preferably of two, amino acids corresponding to positions selected from the group consisting of R181, G182, D183, and G184, preferably D183* and G184*, wherein the numbering is according to the amino acid sequence set forth in SEQ ID NO: 2.

Preferably, the amylase variant comprises a deletion at two amino acids corresponding to positions selected from the group consisting of 181, 182, 183 and 184, preferably a deletion of amino acids corresponding to positions 181 and 182, 182 and 183, or 183 and 184, preferably the deletion D183* and G184*, wherein the numbering is according to SEQ ID NO: 2.

In one embodiment, the amylase variant of a parent amylase comprises
(i) an amino acid substitution at position 25 according to the numbering of SEQ ID NO: 2,
(ii) an amino acid substitution at one or more, preferably two or more, amino acid positions corresponding to amino acid positions selected from the group consisting of 116, 176, 181, 186, 195, 206, 225, 320, and 482 according to the numbering of SEQ ID NO: 2, and
(iii) at least 60%, preferably at least 91% identity, but less than 100% sequence identity with SEQ ID NO: 1-15, preferably of SEQ ID NO: 1 or SEQ ID NO: 2, preferably SEQ ID NO: 1, preferably wherein the parent amylase of the amylase variant is an amylase according to SEQ ID NO: 1 or SEQ ID NO: 2, preferably SEQ ID NO: 1.

Preferably, the amylase variant comprises an amino acid substitution at position 25 and an amino acid substitution at one or more positions selected from the group consisting of 116, 181, 225, and 320 according to the numbering of SEQ ID NO: 2.

Preferably, the amylase variant comprises an amino acid substitution at position 25 and an amino acid substitution at one or more positions selected from the group consisting of 176 and 186 according to the numbering of SEQ ID NO: 2.

Preferably, the amylase variant comprises an amino acid substitution at position 25 and an amino acid substitution at one or more positions selected from the group consisting of 195 or 206 according to the numbering of SEQ ID NO: 2.

Preferably, the amylase variant comprises an amino acid substitution at position 25 and an amino acid substitution at position 482 according to the numbering of SEQ ID NO: 2.

Preferably, said amino acid substitution at position 25 is X25H.

Preferably, said amino acid substitution at position 116 is X116K, preferably said amino acid substitution at position 176 is X176K, preferably said amino acid substitution at position 181 is X181T, preferably said amino acid substitution at position 186 is X186E, preferably said amino acid substitution at position 195 is X195F, preferably said amino acid substitution at position 206 is X206Y, preferably said amino acid substitution at position 225 is X225A, preferably said amino acid substitution at position 320 is X320K, and said amino acid substitution at position 482 is X482W.

Preferably, the amylase variant comprises an amino acid substitution at position 25, preferably X25H, and (according to the numbering of SEQ ID NO: 2)
a) a substitution at one or more positions selected from 176 and 186; preferably one or more substitutions selected from X176K and X186E, or
b) a substitution at one or more positions selected from 116, 181, 225, and 320, preferably one or more substitutions selected from X116K, X225A, and X320K, or
c) a substitution at one or more positions selected from 176 and 186, preferably one or more substitutions selected from X176K and X186E, and a substitution at one or more positions selected from 116, 181, 225, and 320, preferably one or more substitutions selected from X116K, X225A, and X320K.

Preferably, the amylase variant comprises an amino acid substitution at position 25, preferably X25H, and (according to the numbering of SEQ ID NO: 2)
a) a substitution at one or more positions selected from 176 and 186; preferably one or more substitutions selected from X176K and X186E, or
b) a substitution at one or more positions selected from 176 and 186, preferably one or more substitutions selected from X176K and X186E, and a substitution at one or more positions selected from 116, 181, 225, and 320, preferably one or more substitutions selected from X116K, X225A, and X320K.

Preferably, the amylase variant comprises an amino acid substitution at position 25, preferably X25H, and (according to the numbering of SEQ ID NO: 2)
a) a substitution at amino acid position 195, preferably X195F, or a substitution at amino acid position 206, preferably X206Y, and a substitution at one or more positions selected from 176 and 186; preferably one or more substitutions selected from X176K and X186E, or
b) a substitution at amino acid position 195, preferably X195F, or a substitution at amino acid position 206, preferably X206Y, and a substitution at one or more positions selected from 116, 181, 225, and 320, preferably one or more substitutions selected from X116K, X225A, and X320K, or
c) a substitution at amino acid position 195, preferably X195F, or a substitution at amino acid position 206, preferably X206Y, and a substitution at one or more positions selected from 176 and 186, preferably one or more substitutions selected from X176K and X186E, and a substitution at one or more positions selected from 116, 181, 225, and 320, preferably one or more substitutions selected from X116K, X225A, and X320K.

Preferably, the amylase variant comprises an amino acid substitution at position 25, preferably X25H, and (according to the numbering of SEQ ID NO: 2)
a) a substitution at amino acid position 195, preferably X195F, or a substitution at amino acid position 206, preferably X206Y, and a substitution at one or more positions selected from 176 and 186; preferably one or more substitutions selected from X176K and X186E, or
b) a substitution at amino acid position 195, preferably X195F, or a substitution at amino acid position 206, preferably X206Y, and a substitution at one or more positions selected from 176 and 186, preferably one or more substitutions selected from X176K and X186E, and a substitution at one or more positions selected from 116, 181, 225, and 320, preferably one or more substitutions selected from X116K, X225A, and X320K.

Preferably, the amylase variant comprises an amino acid substitution at position 25, preferably X25H, and (according to the numbering of SEQ ID NO: 2)
a) a substitution at amino acid position 195, preferably X195F, and a substitution at one or more positions selected from 176 and 186; preferably one or more substitutions selected from X176K and X186E, or
b) a substitution at amino acid position 195, preferably X195F, and a substitution at one or more positions selected from 176 and 186, preferably one or more substitutions selected from X176K and X186E, and a substitution at one or more positions selected from 116, 181, 225, and 320, preferably one or more substitutions selected from X116K, X225A, and X320K.

Preferably, the amylase variant comprises
(i) the amino acid substitution X25H according to the numbering of SEQ ID NO: 3,
(ii) one or more, two or more, three or more, four or more, five or more, six or more, seven or more, eight or more, or all of the amino acid substitutions selected from the group consisting of X116K, X176K, X181T, X186E, X195F, X206Y, X225A, X320K, and X482W according to the numbering of SEQ ID NO: 3.

Preferably, the present invention is directed to an amylase variant of a parent amylase, wherein said variant comprises
(i) the amino acid substitution X25H according to the numbering of SEQ ID NO: 3,
(ii) the amino acid substitution either X195F or X206Y according to the numbering of SEQ ID NO: 3,
(iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 3.

Preferably, the amylase variant comprises one of the following combinations of amino acid substitutions (according to the numbering of SEQ ID NO: 2):

X25H + X176K + X186E
X25H + X176K + X186E + X195F
X25H + X116K + X181T + X195F + X225A + X320K
X25H + X116K + X176K + X181T + X195F + X225A + X320K
X25H + X116K + X181T + X186E + X195F + X225A + X320K
X25H + X116K + X181T + X195F + X225A + X320K + X482W
X25H + X116K + X176K + X181T + X186E + X195F + X225A + X320K
X25H + X116K + X176K + X181T + X195F + X225A + X320K + X482W
X25H + X116K + X181T + X186E + X195F + X225A + X320K + X482W
X25H + X116K + X176K + X181T + X186E + X195F + X225A + X320K + X482W

Most preferred, the amylase variant comprises one of the following combinations of amino acid substitutions X25H+X116K+X176K+X181T+X186E+X195F+X225A+X320K+X482W (according to the numbering of SEQ ID NO: 2).

Preferably, the amylase variant comprises one of the following combinations of amino acid substitutions (according to the numbering of SEQ ID NO: 2):

X25H + X176K + X186E
X25H + X176K + X186E + X206Y
X25H + X116K + X181T + X206Y + X225A + X320K
X25H + X116K + X176K + X181T + X206Y + X225A + X320K
X25H + X116K + X181T + X186E + X206Y + X225A + X320K
X25H + X116K + X181T + X206Y + X225A + X320K + X482W
X25H + X116K + X176K + X181T + X186E + X206Y + X225A + X320K
X25H + X116K + X176K + X181T + X206Y + X225A + X320K + X482W
X25H + X116K + X181T + X186E + X206Y + X225A + X320K + X482W
X25H + X116K + X176K + X181T + X186E + X206Y + X225A + X320K + X482W

Most preferred, the amylase variant comprises one of the following combinations of amino acid substitutions X25H+X116K+X176K+X181T+X186E+X206Y+X225A+X320K+X482W (according to the numbering of SEQ ID NO: 2).

In one embodiment, the amylase variant of a parent amylase comprises
(i) an amino acid substitution at position 4 according to the numbering of SEQ ID NO: 2,
(ii) an amino acid substitution at one or more, preferably two or more, amino acid positions corresponding to amino acid positions selected from the group consisting of 25, 176, 186, 251, 405, and 482 according to the numbering of SEQ ID NO: 2, and
(iii) at least 60%, preferably at least 91% identity, but less than 100% sequence identity with SEQ ID NO: 1, or SEQ ID NO: 2, or in any of SEQ ID NO: 3-15, preferably of SEQ ID NO: 1 or SEQ ID NO: 2, preferably SEQ ID NO: 1, preferably wherein the parent amylase of the amylase variant is an amylase according to SEQ ID NO: 1 or SEQ ID NO: 2, preferably SEQ ID NO: 1.

Preferably, the amylase variant comprises an amino acid substitution at position 4 and an amino acid substitution at position 25 according to the numbering of SEQ ID NO: 2.

Preferably, the amylase variant comprises an amino acid substitution at position 4 and an amino acid substitution at one or more positions selected from the group consisting of 176 and 186 according to the numbering of SEQ ID NO: 2.

Preferably, the amylase variant comprises an amino acid substitution at position 4 and an amino acid substitution at one or more positions selected from the group consisting of 251 and 405 according to the numbering of SEQ ID NO: 2.

Preferably, the amylase variant comprises an amino acid substitution at position 4 and an amino acid substitution at position 482 according to the numbering of SEQ ID NO: 2.

In one embodiment, the amylase variant of a parent amylase comprises
(i) an amino acid substitution at position 25 and 4 according to the numbering of SEQ ID NO: 2,
(ii) an amino acid substitution at one or more, preferably two or more, amino acid positions corresponding to amino acid positions selected from the group consisting of 176, 186, 251, 405, and 482 according to the numbering of SEQ ID NO: 2, and
(iii) at least 60%, preferably at least 91% identity, but less than 100% sequence identity with SEQ ID NO: 1 or SEQ ID NO: 2, or in any of SEQ ID NO: 3-15, preferably of SEQ ID NO: 1 or SEQ ID NO: 2, preferably SEQ ID NO: 1, preferably wherein the parent amylase of the amylase variant is an amylase according to SEQ ID NO: 1 or SEQ ID NO: 2, preferably SEQ ID NO: 1.

Preferably, said amino acid substitution at position 4 is X4Q. Preferably, said amino acid substitution at position 25 is X25H.

Preferably, said amino acid substitution at position 176 is X176K, preferably said amino acid substitution at position 186 is X186E, preferably said amino acid substitution at position 251 is X251E, preferably said amino acid substitution at position 405 is X405M, and preferably said amino acid substitution at position 482 is X482W.

Preferably, the amylase variant comprises (according to the numbering of SEQ ID NO: 2)
a) a substitution at one or more positions selected from 25, 176, and 186; preferably one or more substitutions selected from X25H, X176K, and X186E, or
b) a substitution at one or more positions selected from 251, 405, and 482, preferably one or more substitutions selected from X251E, X405M, and X482W, or
c) a substitution at one or more positions selected from 25, 176, and 186, preferably one or more substitutions selected from X25H, X176K, and X186E, and a substitution at one or more positions selected from 251, 405, and 482, preferably one or more substitutions selected from X251E, X405M, and X482W.

Preferably, the amylase variant comprises
(i) the amino acid substitution X4G according to the numbering of SEQ ID NO: 2,
(ii) one or more, two or more, three or more, four or more, five or more, or all of the amino acid substitutions selected from the group consisting of X25H, X176K, X186E, X251E, X405M, and X482W according to the numbering of SEQ ID NO: 2.

Preferably, the amylase variant an amino acid substitution at position 4, preferably X4Q, according to the numbering of SEQ ID NO: 2, and
a) a substitution at one or more positions selected from 25, 176, and 186; preferably one or more substitutions selected from X25H, X176K, and X186E, or
b) a substitution at one or more positions selected from 251, 405, and 482, preferably one or more substitutions selected from X251E, X405M, and X482W, or
c) a substitution at one or more positions selected from 25, 176, and 186, preferably one or more substitutions selected from X25H, X176K, and X186E, and a substitution at one or more positions selected from 251, 405, and 482, preferably one or more substitutions selected from X251E, X405M, and X482W.

Preferably, the amylase variant comprises
(i) the amino acid substitution X4G according to the numbering of SEQ ID NO: 2,
(ii) the amino acid substitution X25H according to the numbering of SEQ ID NO: 2, and
(iii) the amino acid substitution X176K and X186E according to the numbering of SEQ ID NO: 2.

Preferably, the amylase variant comprises
(i) the amino acid substitution X4G according to the numbering of SEQ ID NO: 2,
(ii) the amino acid substitution X25H according to the numbering of SEQ ID NO: 2,
(iii) the amino acid substitution X176K and X186E according to the numbering of SEQ ID NO: 2, and
(iv) one or more, two or more, or all of the amino acid substitutions selected from the group consisting of X251E, X405M, and X482W according to the numbering of SEQ ID NO: 2.

Preferably, the amylase variant comprises one of the following combinations of amino acid substitutions (according to the numbering of SEQ ID NO: 2):

X4Q + X176K + X186E
X4Q + X25H + X176K + X186E
X4Q + X25H + X176K + X186E + X251E
X4Q + X25H + X176K + X186E + X405M
X4Q + X25H + X176K + X186E + X482W
X4Q + X25H + X176K + X186E + X251E + X405M
X4Q + X25H + X176K + X186E + X251E + X482W
X4Q + X25H + X176K + X186E + X405M + X482W
X4Q + X25H + X176K + X186E + X251E + X405M + X482W

Most preferred, the amylase variant comprises the following combination of amino acid substitutions: X4Q+X25H+X176K+X186E+X251E+X405M+X482W (according to the numbering of SEQ ID NO: 2).

The amylase variant according to the present invention having one or more amino acid substitutions as described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence of the parent amylase.

Preferably, the amylase variant according to the present invention having one or more of the amino acid substitutions described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 1-15, preferably SEQ ID NO: 1 or 2, preferably SEQ ID NO: 1.

Preferably, the amylase variant according to the present invention having one or more of the amino acid substitutions described herein preferably has at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 1.

Preferably, the amylase variant according to the present invention having one or more of the amino acid substitutions described herein preferably has at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 1.

In another embodiment, the amylase variant according to the present invention having amylase activity comprises or consists of the amino acid sequence set forth in any of SEQ ID NO: 1-15, preferably SEQ ID NO: 1 or SEQ ID NO: 2, preferably SEQ ID NO: 1, with one or more of the herein cited amino acid alterations.

In another embodiment, the amylase variant according to the present invention having amylase activity comprises or consists of the amino acid sequence set forth in any of SEQ ID NO: 1-15, preferably SEQ ID NO: 1 or SEQ ID NO: 2, preferably SEQ ID NO: 1, with the amino acid substitution X25H and, preferably the amino acid substitution either X195F or X206Y according to the numbering of SEQ ID NO: 2, and with 1 to 7, preferably 2 to 7 or 3 to 7, such as 1, 2, 3, 4, 5, 6, or 7 of the amino acid substitutions selected from the group consisting of X116K, X176K, X181T, X186E, X225A, X320K, and X482W according to the numbering of SEQ ID NO: 2.

In another embodiment, the amylase variant according to the present invention having amylase activity comprises or consists of the amino acid sequence set forth in any of SEQ ID NO: 1-15, preferably SEQ ID NO: 1 or SEQ ID NO: 2, preferably SEQ ID NO: 1, with the amino acid substitution X4Q according to the numbering of SEQ ID NO: 2 and with 1 to 6, preferably 2 to 6 or 3 to 6, such as 1, 2, 3, 4, 5, or 6 of the amino acid substitutions selected from the group consisting of X25H, X176K, X186E, X251E, X405M, and X482W according to the numbering of SEQ ID NO: 2.

Preferably, the amylase variant according to the present invention having amylase activity comprises or consists of the amino acid sequence set forth in any of SEQ ID NO: 1-15, preferably SEQ ID NO: 1 or SEQ ID NO: 2, preferably SEQ ID NO: 1, with one or more of the above cited amino acid alterations and further comprises 1 to 50, preferably 1 to 30, 1 to 25, 1 to 20, 1 to 15, 1 to 10, or 1 to 5, 2 to 30, 2 to 25, 2 to 20, 2 to 15 2 to 10, 2 to 8, or 2 to 5, preferably 3 to 30, 3 to 25, 3 to 20, 3 to 15 3 to 10, 3 to 8, or 3 to 5, preferably, 4 to 30, 4 to 25, 4 to 20, 4 to 15, 4 to 10, or 4 to 8 conservative amino acid exchanges, preferably including a deletion at one or more, preferably at two or more, amino acids corresponding to positions selected from the group consisting of 181, 182, 183 and 184 according to the numbering of SEQ ID NO: 2, as described herein.

Preferably, the amylase variant according to the present invention having amylase activity comprises or consists of the amino acid sequence set forth in any of SEQ ID NO: 1-15, preferably SEQ ID NO: 1 or SEQ ID NO: 2, preferably SEQ ID NO: 1, with the amino acid substitution X25H and, preferably the amino acid substitution either X195F or X206Y according to the numbering of SEQ ID NO: 2, and with 1 to 7, preferably 2 to 7 or 3 to 7, such as 1, 2, 3, 4, 5, 6, or 7 of the amino acid substitutions selected from the group consisting of X116K, X176K, X181T, X186E, X225A, X320K, and X482W according to the numbering of SEQ ID NO: 2, and further comprises 1 to 50, preferably 1 to 30, 1 to 25, 1 to 20, 1 to 15, 1 to 10, or 1 to 5, 2 to 30, 2 to 25, 2 to 20, 2 to 15 2 to 10, 2 to 8, or 2 to 5, preferably 3 to 30, 3 to 25, 3 to 20, 3 to 15 3 to 10, 3 to 8, or 3 to 5, preferably, 4 to 30, 4 to 25, 4 to 20, 4 to 15, 4 to 10, or 4 to 8 conservative amino acid exchanges, preferably including a deletion at one or more, preferably at two or more, amino acids corresponding to positions selected from the group consisting of 181, 182, 183 and 184 according to the numbering of SEQ ID NO: 2, as described herein.

Preferably, the amylase variant according to the present invention having amylase activity comprises or consists of the amino acid sequence set forth in SEQ ID NO: 1 with the amino acid substitution X25H and, preferably the amino acid substitution either X195F or X206Y according to the numbering of SEQ ID NO: 2, and with 1 to 7, preferably 2 to 7 or 3 to 7, such as 1, 2, 3, 4, 5, 6, or 7 of the amino acid substitutions selected from the group consisting of X116K, X176K, X181T, X186E, X225A, X320K, and X482W according to the numbering of SEQ ID NO: 2, and further comprises 1 to 10, preferably 1 to 5 conservative amino acid exchanges.

Preferably, the amylase variant according to the present invention having amylase activity comprises or consists of the amino acid sequence set forth in any of SEQ ID NO: 1-15, preferably SEQ ID NO: 1 or SEQ ID NO: 2, preferably SEQ ID NO: 1, with the amino acid substitution X4Q according to the numbering of SEQ ID NO: 2 and with 1 to 6, preferably 2 to 6 or 3 to 6, such as 1, 2, 3, 4, 5, or 6 of the amino acid substitutions selected from the group consisting of X25H, X176K, X186E, X251E, X405M, and X482W according to the numbering of SEQ ID NO: 2, and further comprises 1 to 50, preferably 1 to 30, 1 to 25, 1 to 20, 1 to 15, 1 to 10, or 1 to 5, 2 to 30, 2 to 25, 2 to 20, 2 to 15 2 to 10, 2 to 8, or 2 to 5, preferably 3 to 30, 3 to 25, 3 to 20, 3 to 15 3 to 10, 3 to 8, or 3 to 5, preferably, 4 to 30, 4 to 25, 4 to 20, 4 to 15, 4 to 10, or 4 to 8 conservative amino acid exchanges preferably including a deletion at one or more, preferably at two or more, amino acids corresponding to positions selected from the group consisting of 181, 182, 183 and 184 according to the numbering of SEQ ID NO: 2, as described herein.

Preferably, the amylase variant according to the present invention having amylase activity comprises or consists of the amino acid sequence set forth in SEQ ID NO: 1 with the amino acid substitution X4Q according to the numbering of SEQ ID NO: 2 and with 1 to 6, preferably 2 to 6 or 3 to 6, such as 1, 2, 3, 4, 5, or 6 of the amino acid substitutions selected from the group consisting of X25H, X176K, X186E, X251E, X405M, and X482W according to the numbering of SEQ ID NO: 2, and further comprises 1 to 10, preferably 1 to 5 conservative amino acid exchanges.

Conservative amino acid substitutions may occur over the full length of the sequence of the amylase variant. In one embodiment, such mutations are not pertaining the functional domains of the amylase variant. In one embodiment, conservative mutations are not pertaining the catalytic centers of the amylase variant.

Preferably, the present invention is directed to an amylase variant of a parent amylase, wherein said variant comprises
(i) an amino acid substitution at position 4 according to the numbering of SEQ ID NO: 2,
(ii) an amino acid substitution at one or more, preferably two or more, amino acid positions corresponding to amino acid positions selected from the group consisting of 25, 176, 186, 251, 405, and 482 according to the numbering of SEQ ID NO: 2,
(iii) at least 60%, preferably at least 91% identity, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, preferably wherein the parent amylase of the amylase variant is an amylase according to SEQ ID NO: 1 or SEQ ID NO: 2, preferably SEQ ID NO: 1, and
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the present invention is directed to an amylase variant of a parent amylase, wherein said variant comprises
(i) an amino acid substitution at position 4 according to the numbering of SEQ ID NO: 2,
(ii) an amino acid substitution at one or more, preferably two or more, more preferably three or more, amino acid positions corresponding to amino acid positions selected from the group consisting of 25, 176, 186, 251, 405, and 482 according to the numbering of SEQ ID NO: 2,
(iii) at least 80%, preferably at least 91% identity, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, preferably wherein the parent amylase of the amylase variant is an amylase according to SEQ ID NO: 1 or SEQ ID NO: 2, preferably SEQ ID NO: 1, and
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) the amino acid substitution X4G according to the numbering of SEQ ID NO: 2,
(ii) one or more, two or more, three or more, four or more, five or more, or all of the amino acid substitutions selected from the group consisting of X25H, X176K, X186E, X251E, X405M, and X482W according to the numbering of SEQ ID NO: 2,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, and
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) the amino acid substitution X4G according to the numbering of SEQ ID NO: 2,
(ii) one or more, two or more, three or more, four or more, five or more, or all of the amino acid substitutions selected from the group consisting of X25H, X176K, X186E, X251E, X405M, and X482W according to the numbering of SEQ ID NO: 2,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1, and
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) a combination of amino acid substitutions selected from the group consisting of (according to the numbering of SEQ ID NO: 2):
   X4Q+X176K+X186E,
   X4Q+X25H+X176K+X186E,
   X4Q+X25H+X176K+X186E+X251E,
   X4Q+X25H+X176K+X186E+X405M,
   X4Q+X25H+X176K+X186E+X482W,
   X4Q+X25H+X176K+X186E+X251E+X405M,
   X4Q+X25H+X176K+X186E+X251E+X482W,
   X4Q+X25H+X176K+X186E+X405M+X482W, and
   X4Q+X25H+X176K+X186E+X251E+X405M+X482W,
(ii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1, and
(iii) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) the amino acid substitution X4G according to the numbering of SEQ ID NO: 2,
(ii) one or more, two or more, three or more, four or more, five or more, or all of the amino acid substitutions selected from the group consisting of X25H, X176K, X186E, X251E, X405M, and X482W according to the numbering of SEQ ID NO: 2,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1,
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
(v) wherein the amylase variant exhibits one or more improved property, preferably relative to said parent amylase, preferably relative to the parent amylase set forth in SEQ ID NO: 1 or SEQ ID NO: 2, preferably SEQ ID NO: 1, preferably the amylase variant has an increase in stability, thermostability, storage stability, storage stability in a detergent composition, wash performance, wash performance in a laundry detergent, and/or wash performance in a dish wash detergent, preferably, the improved property is improved stability, preferably storage stability in a detergent composition, preferably wherein said improved property is expressed as an Improvement Factor (IF) of >1.0 and wherein preferably the Improvement Factor is equal or greater than 1.1, preferably, equal or greater than 1.2, more preferably, equal or greater than 1.3.

Particularly preferred, the amylase variant comprises
(i) a combination of amino acid substitutions selected from the group consisting of (according to the numbering of SEQ ID NO: 2):
   X4Q+X176K+X186E,
   X4Q+X25H+X176K+X186E,
   X4Q+X25H+X176K+X186E+X251E,
   X4Q+X25H+X176K+X186E+X405M,
   X4Q+X25H+X176K+X186E+X482W,
   X4Q+X25H+X176K+X186E+X251E+X405M,
   X4Q+X25H+X176K+X186E+X251E+X482W,
   X4Q+X25H+X176K+X186E+X405M+X482W, and
   X4Q+X25H+X176K+X186E+X251E+X405M+X482W,
(ii) at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, and
(iii) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Especially preferred is an amylase variant comprising
(i) the combination of amino acid substitutions (according to the numbering of SEQ ID NO: 2): X4Q+X25H+X176K+X186E+X251E+X405M+X482W,
(ii) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, and
(iii) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Most preferred is an amylase variant comprising or consisting of the amino acid sequence set forth in SEQ ID NO: 1 with the amino acid alterations X4Q+X25H+X176K+X186E+X251E+X405M+X482W according to the numbering of SEQ ID NO: 2.

Preferably, the present invention is directed to an amylase variant of a parent amylase, wherein said variant comprises
(i) an amino acid substitution at position 25 according to the numbering of SEQ ID NO: 2,
(ii) an amino acid substitution at one or more, preferably two or more, amino acid positions corresponding to amino acid positions selected from the group consisting of 116, 176, 181, 186, 195, 206, 225, 320, and 482 according to the numbering of SEQ ID NO: 2,
(iii) at least 60%, preferably at least 91% identity, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, preferably wherein the parent amylase of the amylase variant is an amylase according to SEQ ID NO: 1 or SEQ ID NO: 2, preferably SEQ ID NO: 1, and
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the present invention is directed to an amylase variant of a parent amylase, wherein said variant comprises
(i) an amino acid substitution at position 25 according to the numbering of SEQ ID NO: 2,
(ii) an amino acid substitution at one or more, preferably two or more, amino acid positions corresponding to amino acid positions selected from the group consisting of 116, 176, 181, 186, 195, 206, 225, 320, and 482 according to the numbering of SEQ ID NO: 2,
(iii) at least 80%, preferably at least 91% identity, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, preferably wherein the parent amylase of the amylase variant is an amylase according to SEQ ID NO: 1 or SEQ ID NO: 2, preferably SEQ ID NO: 1, and
(iv) said amylase variant invention comprises a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) the amino acid substitution X25H according to the numbering of SEQ ID NO: 2,
(ii) one or more, two or more, three or more, four or more, five or more, six or more, seven or more, eight or more, or all of the amino acid substitutions selected from the group consisting of X116K, X176K, X181T, X186E, X195F, X206Y, X225A, X320K, and X482W according to the numbering of SEQ ID NO: 2,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, and
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) the amino acid substitution X25H according to the numbering of SEQ ID NO: 2,
(ii) one or more, two or more, three or more, four or more, five or more, six or more, seven or more, eight or more, or all of the amino acid substitutions selected from the group consisting of X116K, X176K, X181T, X186E, X195F, X206Y, X225A, X320K, and X482W according to the numbering of SEQ ID NO: 2,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1, and
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the present invention is directed to an amylase variant of a parent amylase, wherein said variant comprises
(i) an amino acid substitution at position 25 according to the numbering of SEQ ID NO: 2,
(ii) an amino acid substitution at either position 195 or 206 according to the numbering of SEQ ID NO: 2,
(iii) an amino acid substitution at one or more, preferably two or more, amino acid positions corresponding to amino acid positions selected from the group consisting of 116, 176, 181, 186, 225, 320, and 482 according to the numbering of SEQ ID NO: 2,
(iv) at least 60%, preferably at least 91% identity, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, and
(v) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the present invention is directed to an amylase variant of a parent amylase, wherein said variant comprises
(i) the amino acid substitution X25H according to the numbering of SEQ ID NO: 2,
(ii) the amino acid substitution either X195F or X206Y according to the numbering of SEQ ID NO: 2,
(iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
(iv) at least 60%, preferably at least 91% identity, but less than 100% sequence identity with SEQ ID NO: 1 or SEQ ID NO: 2, or in any of SEQ ID NO: 3-15, preferably with SEQ ID NO: 1 or SEQ ID NO: 2, preferably SEQ ID NO: 1, and
(v) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the present invention is directed to an amylase variant of a parent amylase, wherein said variant comprises
(i) the amino acid substitution X25H according to the numbering of SEQ ID NO: 2,
(ii) the amino acid substitution either X195F or X206Y according to the numbering of SEQ ID NO: 2,
(iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
(iv) at least 60%, preferably at least 91% identity, but less than 100% sequence identity with SEQ ID NO: 1, and
(v) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) a combination of amino acid substitutions selected from the group consisting of (according to the numbering of SEQ ID NO: 2):
   X25H+X176K+X186E,
   X25H+X176K+X186E+X195F,
   X25H+X176K+X186E+X206Y,
   X25H+X116K+X181T+X206Y+X225A+X320K,
   X25H+X116K+X176K+X181T+X206Y+X225A+X320K,
   X25H+X116K+X181T+X186E+X206Y+X225A+X320K,
   X25H+X116K+X181T+X206Y+X225A+X320K+X482W,
   X25H+X116K+X176K+X181T+X186E+X206Y+X225A+X320K,
   X25H+X116K+X176K+X181T+X206Y+X225A+X320K+X482W,
   X25H+X116K+X181T+X186E+X206Y+X225A+X320K+X482W,
   X25H+X116K+X176K+X181T+X186E+X206Y+X225A+X320K+X482W,
   X25H+X116K+X181T+X195F+X225A+X320K,
   X25H+X116K+X176K+X181T+X195F+X225A+X320K,
   X25H+X116K+X181T+X186E+X195F+X225A+X320K,
   X25H+X116K+X181T+X195F+X225A+X320K+X482W,
   X25H+X116K+X176K+X181T+X186E+X195F+X225A+X320K,
   X25H+X116K+X176K+X181T+X195F+X225A+X320K+X482W,
   X25H+X116K+X181T+X186E+X195F+X225A+X320K+X482W, and
   X25H+X116K+X176K+X181T+X186E+X195F+X225A+X320K+X482W,
(ii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1, and
(iii) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) a combination of amino acid substitutions selected from the group consisting of (according to the numbering of SEQ ID NO: 2):
   X25H+X176K+X186E+X195F,
   X25H+X116K+X181T+X195F+X225A+X320K,
   X25H+X116K+X176K+X181T+X195F+X225A+X320K,
   X25H+X116K+X181T+X186E+X195F+X225A+X320K,
   X25H+X116K+X181T+X195F+X225A+X320K+X482W,
   X25H+X116K+X176K+X181T+X186E+X195F+X225A+X320K,
   X25H+X116K+X176K+X181T+X195F+X225A+X320K+X482W,
   X25H+X116K+X181T+X186E+X195F+X225A+X320K+X482W, and
   X25H+X116K+X176K+X181T+X186E+X195F+X225A+X320K+X482W,
(ii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1, and
(iii) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) a combination of amino acid substitutions selected from the group consisting of (according to the numbering of SEQ ID NO: 2):
   X25H+X176K+X186E+X206Y,
   X25H+X116K+X181T+X206Y+X225A+X320K,
   X25H+X116K+X176K+X181T+X206Y+X225A+X320K,
   X25H+X116K+X181T+X186E+X206Y+X225A+X320K,
   X25H+X116K+X181T+X206Y+X225A+X320K+X482W,
   X25H+X116K+X176K+X181T+X186E+X206Y+X225A+X320K,
   X25H+X116K+X176K+X181T+X206Y+X225A+X320K+X482W,
   X25H+X116K+X181T+X186E+X206Y+X225A+X320K+X482W, and
   X25H+X116K+X176K+X181T+X186E+X206Y+X225A+X320K+X482W,
(ii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1, and
(iii) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Preferably, the amylase variant comprises
(i) the amino acid substitution X25H according to the numbering of SEQ ID NO: 2,
(ii) one or more, two or more, three or more, four or more, five or more, six or more, seven or more, eight or more, or all of the amino acid substitutions selected from the group consisting of X116K, X176K, X181T, X186E, X195F, X206Y, X225A, X320K, and X482W according to the numbering of SEQ ID NO: 2,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1,
(iv) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
(v) wherein the amylase variant exhibits one or more improved property, preferably relative to said parent amylase, preferably relative to the parent amylase set forth in SEQ ID NO: 1 or SEQ ID NO: 2, preferably SEQ ID NO: 1, preferably the amylase variant has an increase in stability, thermostability, storage stability, storage stability in a detergent composition, wash performance, wash performance in a laundry detergent, and/or wash performance in a dish wash detergent, preferably, the improved property is improved stability, preferably storage stability in a detergent composition, preferably wherein said improved property is expressed as an Improvement Factor (IF) of >1.0 and wherein preferably the Improvement Factor is equal or greater than 1.1, preferably, equal or greater than 1.2, more preferably, equal or greater than 1.3.

Preferably, the amylase variant comprises
(i) the amino acid substitution X25H according to the numbering of SEQ ID NO: 2,
(ii) the amino acid substitution either X195F or X206Y according to the numbering of SEQ ID NO: 2,
(iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
(iv) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1,
(v) a deletion of one or more amino acids corresponding to positions selected from the group consisting of 181, 182, 183 and 184 corresponding to the numbering of SEQ ID NO: 2, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
(vi) wherein the amylase variant exhibits one or more improved property, preferably relative to said parent amylase, preferably relative to the parent amylase set forth in SEQ ID NO: 1 or SEQ ID NO: 2, preferably SEQ ID NO: 1, preferably the amylase variant has an increase in stability, thermostability, storage stability, storage stability in a detergent composition, wash performance, wash performance in a laundry detergent, and/or wash performance in a dish wash detergent, preferably, the improved property is improved stability, preferably wherein said improved property is expressed as an Improvement Factor (IF) of >1.0 and wherein preferably the Improvement Factor is equal or greater than 1.1, preferably, equal or greater than 1.2, more preferably, equal or greater than 1.3.

Particularly preferred, the amylase variant comprises
(i) a combination of amino acid substitutions selected from the group consisting of (according to the numbering of SEQ ID NO: 2):
   X25H+X176K+X186E,
   X25H+X176K+X186E+X195F,
   X25H+X176K+X186E+X206Y,
   X25H+X116K+X181T+X206Y+X225A+X320K,
   X25H+X116K+X176K+X181T+X206Y+X225A+X320K,
   X25H+X116K+X181T+X186E+X206Y+X225A+X320K,
   X25H+X116K+X181T+X206Y+X225A+X320K+X482W,
   X25H+X116K+X176K+X181T+X186E+X206Y+X225A+X320K,
   X25H+X116K+X176K+X181T+X206Y+X225A+X320K+X482W,
   X25H+X116K+X181T+X186E+X206Y+X225A+X320K+X482W,
   X25H+X116K+X176K+X181T+X186E+X206Y+X225A+X320K+X482W,
   X25H+X116K+X181T+X195F+X225A+X320K,
   X25H+X116K+X176K+X181T+X195F+X225A+X320K,
   X25H+X116K+X181T+X186E+X195F+X225A+X320K,
   X25H+X116K+X181T+X195F+X225A+X320K+X482W,
   X25H+X116K+X176K+X181T+X186E+X195F+X225A+X320K,
   X25H+X116K+X176K+X181T+X195F+X225A+X320K+X482W,
   X25H+X116K+X181T+X186E+X195F+X225A+X320K+X482W, and
   X25H+X116K+X176K+X181T+X186E+X195F+X225A+X320K+X482W,
(ii) at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, and
(iii) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Especially preferred is an amylase variant comprising
(i) the combination of amino acid substitutions (according to the numbering of SEQ ID NO: 2): X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably X25H+X116K+X176K+X181T+X186E+X195F+X225A+X320K+X482W,
(ii) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, and
(iii) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).

Much preferred is an amylase variant comprising or consisting of the amino acid sequence set forth in SEQ ID NO: 1 with the amino acid alterations X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably X25H+X116K+X176K+X181T+X186E+X195F+X225A+X320K+X482W according to the numbering of SEQ ID NO: 2.

### Proteases

In one embodiment, the enzyme composition of the present invention comprises a protease. Proteases and protease variants described herein have protease activity (also called “proteolytic activity” herein). This property is related to the hydrolytic activity of a protease (proteolysis, which means hydrolysis of peptide bonds linking amino acids together in a polypeptide chain) on protein containing substrates, e.g. casein, haemoglobin and BSA. Quantitatively, proteolytic activity is related to the rate of degradation of proteins by a protease or proteolytic enzyme in a defined course of time. The methods for analyzing proteolytic activity are well-known in the literature (see e.g. Gupta et al. (2002), Appl. Microbiol. Biotechnol. 60: 381-395).

For instance, proteolytic activity can be determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage, resulting in release of free pNA which has a yellow color that can be quantified by measuring the optical density at 405 nm (OD405).

The protease of the enzyme composition is selected from
a protease variant of a parent protease, wherein the protease variant comprises an amino acid sequence comprising compared to SEQ ID NO: 16 at least two additional negative charges in the loop region of residues 98 to 104 according to the numbering of SEQ ID NO: 17, and an amino acid sequence which is at least 60%, but less than 100% identical to SEQ ID NO: 16-34, preferably SEQ ID NO: 16, or
a protease variant of a parent protease, wherein the protease variant comprises
   (i) an amino acid substitution at one or more amino acid positions corresponding to amino acid positions selected from the group consisting of 3, 4, 9, 19, 22, 43, 62, 63, 76, 87, 99, 101, 103, 104, 116, 118, 128, 129, 130, 156, 160, 167, 170, 182, 185, 188, 194, 205, 213, 217, 218, 232, 245, 256, 259, 262, and 271 according to the numbering of SEQ ID NO: 17 and an amino acid sequence which is at least 60%, but less than 100% sequence identity with SEQ ID NO: 22, or
   (ii) an amino acid substitution at one or more amino acid positions corresponding to amino acid positions selected from the group consisting of 24, 53, 76, 78, 101, 128, and 217 according to the numbering of SEQ ID NO: 17 and an amino acid sequence which is at least 60%, but less than 100% sequence identity with SEQ ID NO: 17, or
   (iii) an amino acid substitution at one or more amino acid positions corresponding to amino acid positions selected from the group consisting of 9, 89, 130, 133, 144, 159, 183, 189. 217, 224, 238, 243, 252, 259, 270 and 271 according to the numbering of SEQ ID NO: 19 and an amino acid sequence which is at least 60%, but less than 100% sequence identity with SEQ ID NO: 19, or
   (iv) an amino acid sequence which is at least 60% sequence identity with SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31.

Preferably, the protease of the enzyme composition is a protease variant of a parent protease, wherein the protease variant comprises an amino acid sequence comprising compared to SEQ ID NO: 16 at least two additional negative charges in the loop region of residues 98 to 104 according to the numbering of SEQ ID NO: 17, and an amino acid sequence which is at least 60%, but less than 100% identical to SEQ ID NO: 16-34, preferably SEQ ID NO: 16.

Preferably, the protease is a variant protease of the parent protease which is preferably a subtilisin protease.

Preferably, the parent protease for the protease variant is a protease having at least 60% sequence identity to any of SEQ ID NO: 16-34, preferably the parent protease for the protease variant is protease according to any of SEQ ID NO: 16-34. Most preferably the parent protease for the protease variant is protease according to SEQ ID NO: 16.

Preferably, in this embodiment, the amino acid residue in the parent protease at the above cited positions (i.e., X) corresponds to the amino acid residue shown in SEQ ID NO: 16 at the respective position according to the numbering of SEQ ID NO: 17.

The variant protease comprises compared to SEQ ID NO: 16 at least two additional negative charges in the loop region of residues 98 to 104 according to the numbering of SEQ ID NO: 17 (BPN′ numbering, i.e., wherein the positions are numbered by their correspondence to the amino acid sequence of subtilisin BPN′ of B. amyloliquefaciens, established as SEQ ID NO: 17).

Preferably, the protease variant comprises as a catalytic triad the amino acids aspartate, histidine and serine, preferably the protease is a subitilisin protease.

Preferably, the at least two additional negative charges in the loop region of residues 98 to 104 according to the numbering of SEQ ID NO: 17 are obtained by one or more amino acid alterations selected from the group consisting of substitutions, deletions and insertions, preferably by substitutions or insertions, preferably substitutions.

Preferably, in the protease the at least two additional negative charges compared to SEQ ID NO: 16 in the loop region of residues 98 to 104 are caused by one or more amino acid substitutions at amino acid position according to the numbering of SEQ ID NO: 17 selected from the group consisting of 98, 99, 100, 101, 102, 103, and 104, preferably at position 101.

Preferably, the at least two additional negative charges in the loop region of residues 98 to 104 according to the numbering of SEQ ID NO: 17 are obtained by one or more amino acid alterations selected from the group consisting of X99E, X101D and X101E, preferably, D99E, R101D and R101E.

In a preferred embodiment, the protease comprises an amino acid sequence which comprises compared to SEQ ID NO: 16 the amino acid substitution R101E or R101D according to the numbering of SEQ ID NO: 17. In another embodiment, the at least two additional negative charges compared to SEQ ID NO: 16 in the loop region of residues 98 to 104 are not caused by the amino acid substitution X101E or X101D. Most preferred, the protease comprises an amino acid sequence which comprises compared to SEQ ID NO: 16 the amino acid substitution R101E according to the numbering of SEQ ID NO: 17.

In a preferred embodiment, the loop sequence 98-104 has compared to SEQ ID NO: 16 two additional negative charges with the following sequence ADGEGAI, ADGDGAI, ADGDGSV, ADGEGSV, AADGSGSV, AADGEGSV, or ASEGEGSV with longer sequences having an insertion in the loop sequence.

In a further preferred embodiment, the amino acid sequence of the protease comprises compared to SEQ ID NO: 16 alanine at position 103 (103A) and isoleucine at position 104 (1041) according to the numbering of SEQ ID NO: 17, more preferably, 101R, 1041, and 103A.

In a further preferred embodiment, the amino acid sequence of the protease compared to SEQ ID NO: 16 does not comprises an additional amino acid residue in the loop region from position 98 to 104 according to the numbering of SEQ ID NO: 17. Preferably, the amino acid sequence of the protease compared to SEQ ID NO: 16 does not comprises an additional amino acid residue between positions 42-43, 51-55, 155-165, 187-189, 217-218, or 218-219 according to the numbering of SEQ ID NO: 17.

In a preferred embodiment, the protease comprises compared to SEQ ID NO: 16 one or more further substitutions at one or more position selected from the group consisting of 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252, and 274 according the numbering of SEQ ID NO: 17.

Preferably, the protease further comprises one or more of the amino acid substitutions selected from the group consisting of X3T, X4I, X156D, X205I, and X262E, according to the numbering of SEQ ID NO: 17.

Preferably, the protease further comprises one or more of the amino acid substitutions selected from the group consisting of S3T, V4I, S156D, V205I, and L262E, according to the numbering of SEQ ID NO: 17.

Preferably, the protease further comprises one or more, preferably all, of the amino acid substitutions selected from the group consisting of X3T, X4I, and X205I according to the numbering of SEQ ID NO: 17.

Preferably, the protease further comprises one or more, preferably all, of the amino acid substitutions selected from the group consisting of S156D and L262E according to the numbering of SEQ ID NO: 17.

Preferably, the protease has at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with any of SEQ ID NO: 16-34.

Preferably, the protease has at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16.

Preferably, the protease variant has at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 at least two additional negative charges in the loop region of residues 98 to 104 according to the numbering of SEQ ID NO: 17.

Preferably, the protease variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 at least two additional negative charges in the loop region of residues 98 to 104 according to the numbering of SEQ ID NO: 17.

Preferably, the protease variant has at least 80%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 the amino acid substitution X101D or X101E, preferably X101E, preferably R101E, according to the numbering of SEQ ID NO: 17.

Preferably, the protease variant has at least 80%, at least 85%, at least 90%, at least 95%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 the amino acid substitution X101E, preferably R101E, according to the numbering of SEQ ID NO: 17.

Preferably the variant protease comprises an amino acid sequence which is at least 80% identical to SEQ ID NO: 16 and wherein the amino acid sequence of the protease comprises compared to SEQ ID NO: 16 at least two additional negative charges in the loop region of residues 98 to 104 according to the numbering of SEQ ID NO: 17 and the protease comprises compared to SEQ ID NO: 16 one or more substitutions at positions according the numbering of SEQ ID NO: 17 selected from the group consisting of 3, 4, 9, 15, 24, 27, 33, 36, 45, 55, 57, 58, 59, 61, 68, 76, 77, 87, 95, 96, 97, 98, 99, 100, 101, 102, 103, 104, 106, 117, 118, 120, 123, 124, 128, 129, 130, 131, 136, 137, 143, 154, 156, 160, 161, 163, 167, 170, 171, 172, 185, 194, 195, 199, 205, 206, 209, 217, 218, 222, 224, 232, 235, 236, 238, 244, 245, 248, 252, 261, 262, and 274. In another embodiment of the present invention, the protease comprising as described herein an amino acid sequence which is at least 80% identical to SEQ ID NO: 16 and wherein the amino acid sequence of the protease comprises compared to SEQ ID NO: 16 at least two additional negative charges in the loop region of residues 98 to 104, the amino acid substitution X101E or X101 D, preferably X101E, according to the numbering of SEQ ID NO: 17, further comprises according to the numbering of SEQ ID NO: 17 at least one of the amino acid residues selected from the group consisting of
a. threonine or serine at position 3 (X3T or X3S),
b. isoleucine or valine at position 4 (X4I or X4V),
c. serine, alanine, threonine or arginine at position 63 (X63S, X63A, X63T or X63R),
d. threonine, aspartic acid or glutamic acid at position 156 (X156T, X156D, or X156E),
e. serine or proline at position 194 (X194S or X194P),
f. serine, valine, or methionine at position 199 (X199S, X199V, or X199M)
g. isoleucine or valine at position 205 (X205I or X205V); and
h. aspartic acid, glutamic acid, glutamine, glycine at position or leucine at position 217 (X217D, X217E, X217Q, X217G or X217L).

Preferably, the protease has at least 80%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 the amino acid substitution X101D or X101E, preferably X101E, and one or more of the amino acid substitutions X3T, X4I, and X205I, preferably S3T, V4I, and V205I according to the numbering of SEQ ID NO: 17. Further preferred, the protease has at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 the amino acid substitution R101D or R101E, preferably R101E, and the amino acid substitutions S3T, V4I, and V205I according to the numbering of SEQ ID NO: 17.

In another embodiment, the protease comprises an amino acid sequence which is at least 80% identical to SEQ ID NO: 16 and the protease comprises compared to SEQ ID NO: 16 the amino acid substitution X101E or X101D, preferably X101E, and one or more substitutions selected from the group consisting of X156D, X262E, X137H, X3T, X45E,D,Q, X55N, X58W,Y,L, X59D,M,N,T, X61D, R, X87E, X97S, X98D,E, R, X106A,W, X117E, X120V,D,K,N, X125M, X129D, X136Q, X144W, X161T, X163A,G, X171L, X172S, X185Q, X199M, X209W, X222Q, X238H, X244T, X261T,D and X262N,Q,D according to the numbering of SEQ ID NO: 17.

In one embodiment, the protease comprises an amino acid sequence which is at least 80% identical to SEQ ID NO: 16 and the protease comprises compared to SEQ ID NO: 16 the amino acid substitution X101E or X101 D, preferably X101E, and one, preferably both, of the substitutions selected from the group consisting of X156D and X262E according to the numbering of SEQ ID NO: 17.

In another embodiment, the protease comprises an amino acid sequence which is at least 80% identical to SEQ ID NO: 16 and the protease comprises compared to SEQ ID NO: 16 the amino acid substitution R101E or R101D, preferably R101E, and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171L, A172S, N185Q, V199M, Y209W, M222Q, N238H, V244T, N261T,D and L262N,Q,D according to the numbering of SEQ ID NO: 17.

In one embodiment, the protease comprises an amino acid sequence which is at least 80% identical to SEQ ID NO: 16 and the protease comprises compared to SEQ ID NO: 16 the amino acid substitution R101E or R101D, preferably R101E, and one, preferably both, of the substitutions selected from the group consisting of S156D and L262E according to the numbering of SEQ ID NO:17.

Further preferred, the protease has at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 the amino acid substitution R101D or R101E, preferably R101E, and the amino acid substitutions S156D and L262E according to the numbering of SEQ ID NO: 17.

Proteases with substitutions as described above are shown for instance in SEQ ID NO: 28.

Thus, in one embodiment the protease variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 28.

In another embodiment, the protease comprises an amino acid sequence which is at least 80% identical to SEQ ID NO: 16 and the protease comprises compared to SEQ ID NO: 16 the amino acid substitutions R101E and one or more, preferably both, of S156D and L262E, and optionally at least one further mutation selected from I104T, H120D, Q137H, S141H, R145H and S163G according to the numbering of SEQ ID NO: 17.

Preferably, the protease has at least 80% sequence identity with SEQ ID NO: 16 and comprises compared to SEQ ID NO: 16 at least two additional negative charges in the loop region of residues 98 to 104 according to the numbering of SEQ ID NO: 17, wherein compared to SEQ ID NO: 16 the protease comprises one or more conservative amino acid exchanges as described herein. Preferably, compared to SEQ ID NO: 16 the protease comprises at least 1, at least 2, at least 3, at least 4, at least 5, at least 6, at least 10, at least 15, at least 20, at least 30 or at least 40 conservative amino acid exchanges.

Preferably, compared to SEQ ID NO: 16 a protease described herein can comprise 1, 2, 3, 4, 5, 6, 7, 8, 9, or 10 conservative amino acid exchanges in addition to the modifications resulting in at least two additional negative charges in the loop region of residues 98 to 104, preferably in addition to the substitution X101D or X101E, preferably X101E, and preferably the amino acid substitutions X3T, X4I, and X205I or the amino acid substitutions X156D and X262E the according to the numbering of SEQ ID NO: 17.

Preferably, the protease has at least 80% sequence identity with SEQ ID NO: 16 and comprises compared to SEQ ID NO: 16 at least two additional negative charges in the loop region of residues 98 to 104 according to the numbering of SEQ ID NO: 17, wherein compared to SEQ ID NO: 16 the remaining difference in amino acid sequence is due to conservative amino acid exchanges as described herein.

Preferably, the protease is a protease variant comprising the amino acid substitution X101E and optional the amino acid substitutions X3T, X4I, and X205I or the amino acid substitutions X156D and X262E according to the numbering of SEQ ID NO: 17, and an amino acid sequence which is at least 80%, but less than 100% identical to SEQ ID NO: 16.

Most preferably, the protease is a protease variant comprising the amino acid substitution X101E according to the numbering of SEQ ID NO: 17, and an amino acid sequence which is at least 80%, but less than 100% identical to SEQ ID NO: 16.

Most preferred is a protease variant comprising or consisting of the amino acid sequence set forth in SEQ ID NO: 16 with the amino acid substitution R101E according to the numbering of SEQ ID NO: 17, i.e., only one amino acid exchange compared to SEQ ID NO: 16.

Alternatively preferred is a protease variant comprising or consisting of the amino acid sequence set forth in SEQ ID NO: 16 with the amino acid substitution R101E and S3T, V4I, and V205I according to the numbering of SEQ ID NO: 17.

Alternatively preferred is a protease variant comprising or consisting of the amino acid sequence set forth in SEQ ID NO: 16 with the amino acid substitution R101E and S156D and L262E according to the numbering of SEQ ID NO: 17.

In one embodiment the protease variant of the enzyme composition comprises
(i) an amino acid substitution at one or more amino acid positions corresponding to amino acid positions selected from the group consisting of 3, 4, 9, 19, 22, 43, 62, 63, 76, 87, 99, 101, 103, 104, 116, 118, 128, 129, 130, 156, 160, 167, 170, 182, 185, 188, 194, 205, 213, 217, 218, 232, 245, 256, 259, 262, and 271 according to the numbering of SEQ ID NO: 17 and an amino acid sequence which has at least 60%, but less than 100% sequence identity with SEQ ID NO 22, or
(ii) an amino acid substitution at one or more amino acid positions corresponding to amino acid positions selected from the group consisting of 24, 53, 76, 78, 101, 128, and 217 according to the numbering of SEQ ID NO: 17 and an amino acid sequence which has at least 60%, but less than 100% sequence identity with SEQ ID NO: 17, or
(iii) an amino acid substitution at one or more amino acid positions corresponding to amino acid positions selected from the group consisting of 9, 89, 130, 133, 144, 159, 183, 189. 217, 224, 238, 243, 252, 259, 270 and 271 according to the numbering of SEQ ID NO: 19 and an amino acid sequence which has at least 60%, but less than 100% sequence identity with SEQ ID NO: 19, or
(iv) an amino acid sequence which is at least 60% sequence identity with SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31.

In one embodiment, the protease variant comprises an amino acid sequence which has at least 60%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 22 and an amino acid substitution at one or more amino acid positions corresponding to amino acid positions selected from the group consisting of 3, 4, 9, 19, 22, 43, 62, 63, 76, 87, 99, 101, 103, 104, 116, 118, 128, 129, 130, 156, 160, 167, 170, 182, 185, 188, 194, 205, 213, 217, 218, 232, 245, 256, 259, 262, and 271 according to the numbering of SEQ ID NO: 17.

Preferably, the protease variant comprises an amino acid sequence which has at least 60%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 22 and one or more amino acid substitutions selected from the group consisting of X3T, X4I, X9D/E/R, X19L, X22R/A, X43R, X62D, X63D/E, X76D, X87N, X99A/D, X101M/R/E, X103A, X1041, X116L, X118V, X128L, X129Q, X130A, X156D/E, X160D/S, X167A, X170S, X182E/D, X185E/D, X188D, X194P, X205I, X213A, X217E/D/Q, X218D, X232V, X245R, X256D/E, X259D/E, X262D/E, and X271F according to the numbering of SEQ ID NO: 17.

Preferably, the protease variant comprises an amino acid sequence which has at least 60%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 22 and one or more amino acid substitutions selected from the group consisting of S3T, V4I, S9D/E/R, R19L, T22R/A, N43R, N62D, G63D/E, N76D, S87N, S99A/D, S101M/R/E, S103A, V104I, N116L, G118V, S128L, P129Q, S130A, S156D/E, G160D/S, Y167A, R170S, Q182E/D, N185E/D, S188D, A194P, V205I, T213A, L217E/D/Q, N218D, A232V, N245R, S256D/E, S259D/E, L262D/E, and E271F according to the numbering of SEQ ID NO: 17.

In a preferred embodiment, the protease variant described above further comprises an insertion of D and E directed adjacent C-terminally of the position 99 according to the numbering of SEQ ID NO: 17.

Proteases with substitutions as described above are shown for instance in SEQ ID NO: 24, SEQ ID NO: 23, SEQ ID NO: 26, SEQ ID NO: 27, and SEQ ID NO: 29.

Thus, in one embodiment the protease variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to any of SEQ ID NO: 24, SEQ ID NO: 23, SEQ ID NO: 26, SEQ ID NO: 27, or SEQ ID NO: 29.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to any of SEQ ID NO: 24, SEQ ID NO: 23, SEQ ID NO: 26, SEQ ID NO: 27, or SEQ ID NO: 29.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 24.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 23.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 26.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 27.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 29.

In another embodiment, the protease variant comprises an amino acid sequence which has at least 60%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 17 and an amino acid substitution at one or more amino acid positions corresponding to amino acid positions selected from the group consisting of 24, 53, 76, 78, 101, 128, and 217 according to the numbering of SEQ ID NO: 17.

Preferably, the protease variant comprises an amino acid sequence which has at least 60%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 17 and one or more of the substitutions selected from the group of X24G/R, X53G, X76D, X78N, X101N, X128A/S, and X217Q/L/E/D according to the numbering of SEQ ID NO:17.

Preferably, the protease variant comprises an amino acid sequence which has at least 60%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 17 and one or more of the substitutions selected from the group of S24G/R, S53G, N76D, S78N, S101N, G128A/S, and Y217Q/L/E/D according to the numbering of SEQ ID NO:17.

Proteases with substitutions as described above are shown for instance in SEQ ID NO: 32, 33, and 34.

Thus, in one embodiment the protease variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to any of SEQ ID NO: 32, 33, or 34.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 32.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 33.

In one embodiment the protease variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 34.

In one embodiment the protease variant comprises an amino acid sequence which has at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with SEQ ID NO: 19 and an amino acid substitution at one or more amino acid positions corresponding to amino acid positions selected from the group consisting of 9, 89, 130, 133, 144, 159, 183, 189. 217, 224, 238, 243, 252, 259, 270 and 271 according to the numbering of SEQ ID NO: 19, preferably one or more, preferably all substitutions selected from the group consisting of X159T, X183S, X238N, X243N, X259N and X270A, more preferably one or more, preferably all substitutions selected from the group consisting of X9T, X130D, X133A, X144K, X159T, X183S, X189T, X217M, X224A, X238N, X243N, X252T, X259N, X270A and X271E, even more preferably one or more, preferably all substitutions selected from the group consisting of X9T, X89A, X130D, X133A, X144K, X159T, X183S, X189T, X217M, X224A, X238N, X243N, X252T, X259N, X270A and X271E.

In one embodiment the protease variant comprises an amino acid sequence which is at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with SEQ ID NO: 19 and one or more, preferably all substitutions selected from the group consisting of X159T, X183S, X238N, X243N, X259N and X270A, more preferably one or more, preferably all substitutions selected from the group consisting of X9T, X130D, X133A, X144K, X159T, X183S, X189T, X217M, X224A, X238N, X243N, X252T, X259N, X270A and X271E, even more preferably one or more, preferably all substitutions selected from the group consisting of X9T, X89A, X130D, X133A, X144K, X159T, X183S, X189T, X217M, X224A, X238N, X243N, X252T, X259N, X270A and X271E according to the numbering of SEQ ID NO: 19.

In one embodiment the protease variant comprises an amino acid sequence which is at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with SEQ ID NO: 19 and one or more, preferably all substitutions selected from the group consisting of F159T, N183S, Y238N, T243N, D259N and V270A, more preferably one or more, preferably all substitutions selected from the group consisting of P9T, N130D, T133A, N144K, F159T, N183S, S189T, Y217M, S224A, Y238N, T243N, N252T, D259N, V270A and Q271E, even more preferably one or more, preferably all substitutions selected from the group consisting of P9T, S89A, N130D, T133A, N144K, F159T, N183S, S189T, Y217M, S224A, Y238N, T243N, N252T, D259N, V270A and Q271E according to the numbering of SEQ ID NO: 19.

In one embodiment, the protease variant has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to any of the amino acid sequences shown in SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31.

Preferably, the protease variant has at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to any of the amino acid sequences shown in SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31. Preferably, the protease variant has at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to any of the amino acid sequences shown in SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31.

In one embodiment, the protease according to the present invention preferably has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 22.

In one embodiment, the protease variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 21.

In one embodiment, the protease variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 30.

In one embodiment, the protease variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 20.

In one embodiment, the protease variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 25.

In one embodiment, the protease variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 18.

In one embodiment, the protease according to the present invention preferably has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 19.

In one embodiment, the protease variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 31.

### Mannanases

In one embodiment, the enzyme composition of the present invention comprises a mannanase. The mannanases and mannanase variants described herein have mannanase activity. “Mannanase” as described herein are enzymes selected from the group of (galacto-)mannan degrading enzyme. The (galacto-)mannan degrading enzyme may be selected from β-mannosidases (EC 3.2.1.25), endo-1,4-β-mannosidases (EC 3.2.1.78), and 1,4-β-mannobiosidases (EC 3.2.1.100). Preferably, the mannan degrading enzyme is selected from the group of endo-1, 4-mannanases, preferably endo-1,4-β-mannosidases (EC 3.2.1.78), a group of enzymes which may be called endo-β-1,4-D-mannanases, β-mannanases, or mannanases herein. Endo-1, 4-mannanases randomly hydrolyze the 1,4-linkages within the mannan backbone releasing manno-oligosaccharides (MOS).

The mannanase may be selected from alkaline mannanases of family 5 or 26 (i.e., GH5 or GH26). The term “alkaline mannanase” is meant to encompass mannanases having an enzymatic activity of at least 40% of their maximum activity at a given pH ranging from 7 to 12, preferably 7.5 to 10.5.

The mannanase of the enzyme composition is selected from
a mannanase variant of a parent mannanase, wherein the mannanase variant comprises one or more amino acid substitution selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and an amino acid sequence which is at least 60%, but less than 100% identical to a sequence according to positions 31-490 of SEQ ID NO: 35 and
a mannanase variant of a parent mannanase, wherein the mannanase variant comprises
   (i) an amino acid substitution at one or more amino acid positions corresponding to amino acid positions selected from the group consisting of 24, 123, and 256 according to the numbering of SEQ ID NO: 39 and an amino acid sequence which is at least 60%, but less than 100% identical to SEQ ID NO: 39, or
   (ii) an amino acid substitution at one or more amino acid positions corresponding to amino acid positions selected from the group consisting of 10, 19, 30, 38, 59, 60, 67, 97, 103, 129, 143, 167, 168, 184, 225, 228, 235, 244, 258, 261, and 298 according to the numbering of SEQ ID NO: 38 and an amino acid sequence which is at least 60%, but less than 100% identical to SEQ ID NO: 38, or
   (iii) an amino acid sequence which is at least 60% identical to SEQ ID NO: 40, or
   (iv) an amino acid substitution at one or more amino acid positions corresponding to amino acid positions selected from the group consisting of 30, 81, 93, 95, 118, 138, 139, 200, 202, 210, 260, 283, 288, 294, and 295 according to the numbering of SEQ ID NO: 40 and an amino acid sequence which is at least 60%, but less than 100% identical to SEQ ID NO: 40.

Preferably, the mannanase of the enzyme composition is a mannanase variant of a parent mannanase, wherein the mannanase variant comprises an amino acid substitution at one or more amino acid positions selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35.

Preferably, the mannanase of the enzyme composition is a mannanase variant of a parent mannanase, wherein the mannanase variant comprises an amino acid substitution at one or more amino acid positions selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and an amino acid sequence which is at least 60%, but less than 100% sequence identity with any of SEQ ID NO: 35-37.

Preferably, the parent mannanase for the mannanase variant is a mannanase having at least 60% sequence identity to any of SEQ ID NO: 35-37, preferably the parent mannanase for the mannanase variant is mannanase according to any of SEQ ID NO: 35-37. Most preferably the parent mannanase for the mannanase variant is mannanase according to SEQ ID NO: 35. Preferably, in this embodiment, the amino acid residue in the parent protease at the above cited positions (i.e., X) corresponds to the amino acid residue shown in SEQ ID NO: 35 at the respective position according to the numbering of SEQ ID NO: 35.

Preferably, the mannanase variant comprises one or more amino acid substitution selected from the group consisting of X86N, X89V/L, X96D, X101T/V, X103Y/E/A, X107N, X108G, X109Q/A, X112N, X119Y/H/T, X122S, X124E/C/D, X126E, X127A, X129M/L/F, X231Q/K/L/P/Y, X233V, X235H/R/L/Q/N/Y, X244I/V/N, X254W, X255Y/H/R, X264Q/V, X270T, X272I, X273T, X274E/C/Q, X281L, X286E/L/Q/A, X289F/M/H, X290A, X296H/F/Y, X301E/C/T, X309L, X312F/Y, X314P, X317T, and X319D/E according to the numbering of SEQ ID NO: 35.

Preferably, the mannanase variant comprises one or more amino acid substitution selected from the group consisting of D86N, Q89V/L, N96D, L101T/V, S103Y/E/A, K107N, N108G, N109Q/A, A112N, A119Y/H/T, N122S, A124E/C/D, S126E, S127A, N129M/L/F, S231Q/K/L/P/Y, 1233V, S235H/R/L/Q/N/Y, D244I/V/N, H254W, K255Y/H/R, E264Q/V, S270T, Q272I, K273T, N274E/C/Q, S281L, G286E/L/Q/A, W289F/M/H, S290A, N296H/F/Y, D301E/C/T, T309L, N312F/Y, A314P, L317T, A319D/E according to the numbering of SEQ ID NO: 35.

Preferably, the mannanase further comprises an amino acid substitution at one or more amino acid positions selected from the group consisting of 328, 329, 330, 331, 344, 359, 374, 416, and 432 according to the numbering of SEQ ID NO: 35.

Preferably, the mannanase variant further comprises one or more amino acid substitution selected from the group consisting of X3281/Q/V, X329L/S/V/T, X330P/D/T, X331A/Q, X344Q/F/T, X359R/Y/C/Q, X374G/V/A/R/N/P, X416W, and X432P/N/L/R/S/T/G/H/I according to the numbering of SEQ ID NO: 35.

Preferably, the mannanase variant further comprises one or more amino acid substitution selected from the group consisting of D3281/Q/V, G329L/S/V/T, G330P/D/T, D331A/Q, Y344Q/F/T, M359R/Y/C/Q, Y374G/V/A/R/N/P, L416W, and W432P/N/L/R/S/T/G/H/I according to the numbering of SEQ ID NO: 35.

Preferably, the mannanase variant comprises an amino acid substitution at one or more amino acid positions selected from the group consisting of 31, 89, 96, 119, 264, 289, 312, 348, 349, 352, and 379 according to the numbering of SEQ ID NO: 35.

Preferably, the mannanase variant comprises amino acid substitutions at position 89 and 96 and an amino acid substitution at one or more amino acid positions selected from the group consisting of 31, 119, 264, 289, and 312 according to the numbering of SEQ ID NO: 35.

Preferably, the mannanase variant comprises an amino acid substitution at one or more amino acid positions selected from the group consisting of 89, 96, 119, 264, 289, 312, 348, 349, and 352 according to the numbering of SEQ ID NO: 35.

Preferably, the mannanase variant comprises one or more amino acid substitution selected from the group consisting of X31V, X89V, X96D, X119Y/H/T, X264Q/V, X289F/M/H, X312F/Y, X348S/R/N/M/G, X349T/S/D/G, X352N/G, and X379V according to the numbering of SEQ ID NO: 35.

Preferably, the mannanase variant comprises at least one of amino acid substitutions X89V and X96D and one or more amino acid substitutions selected from X31V, X119Y/H/T, X264Q/V, X289F/M/H, and X312F/Y according to the numbering of SEQ ID NO: 35.

Preferably, the mannanase variant comprises one or more amino acid substitution selected from the group consisting of A31V, Q89V, N96D, A119Y/H/T, E264Q/V, W289F/M/H, N312F/Y, T348S/R/N/M/G, E349T/S/D/G, S352N/G, D379V according to the numbering of SEQ ID NO: 35.

Preferably, the mannanase comprises at least one of amino acid substitution Q89V and N96D and one or more amino acid substitutions selected from A31V, A119Y/H/T, E264Q/V, W289F/M/H, and N312F/Y according to the numbering of SEQ ID NO: 35.

More preferably, the mannanase variant comprises at least one, at least two, at least three, at least four, at least five, at least six, at least seven, at least eight or all amino acid substitutions X89V, X96D, X119Y/H/T, X264Q/V, X289F/M/H, X312F/Y, X348S/R/N/M/G, X349T/S/D/G, and X352N/G according to the numbering of SEQ ID NO: 35.

More preferably, the mannanase variant comprises at least one, at least two, at least three, at least four, at least five, at least six, at least seven, at least eight or all amino acid substitutions Q89V, N96D, A119Y/H/T, E264Q/V, W289F/M/H, N312F/Y, T348S/R/N/M/G, E349T/S/D/G, and S352N/G according to the numbering of SEQ ID NO: 35.

More preferably, the mannanase variant comprises at least one, at least two, at least three, at least four, at least five, at least six, at least seven, at least eight or all amino acid substitutions X89V, X96D, X119Y/H/T, X264Q/V, X289F/M/H, X312F/Y, X348S/R/N/M/G, X349T/S/D/G, and X352N/G according to the numbering of SEQ ID NO: 35.

More preferably, the mannanase variant comprises at least one, at least two, at least three, at least four, at least five, at least six, at least seven, at least eight or all amino acid substitutions Q89V, N96D, A119Y/H/T, E264Q/V, W289F/M/H, N312F/Y, T348S/R/N/M/G, E349T/S/D/G, and S352N/G according to the numbering of SEQ ID NO: 35.

More preferably, the mannanase variant comprises at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35.

More preferably, the mannanase variant comprises at least one, at least two, at least three, at least four, at least five, at least six, at least seven, at least eight or all amino acid substitutions Q89V, N96D, A119H, E264Q, W289M, N312Y, T348N, E349G, and S352G according to the numbering of SEQ ID NO: 35.

More preferably, the mannanase variant comprises the amino acid substitutions X89V, X96D, X119H, X264Q, X289M, and X312Y according to the numbering of SEQ ID NO: 35.

More preferably, the mannanase variant comprises the amino acid substitutions Q89V, N96D, A119H, E264Q, W289M, and N312Y according to the numbering of SEQ ID NO: 35.

Most preferably, the mannanase variant comprises the amino acid substitutions X89V, X96D, X119H, X264Q, X289M, X312Y, X348N, X349G, and X352G according to the numbering of SEQ ID NO: 35.

Most preferably, the mannanase variant comprises the amino acid substitutions Q89V, N96D, A119H, E264Q, W289M, N312Y, T348N, E349G, and S352G according to the numbering of SEQ ID NO: 35.

Preferably, the mannanase variant further comprises an amino acid substitution at one or more amino acid positions selected from the group consisting of 341, 346, 348, 349, 352, 356, and 379 according to the numbering of SEQ ID NO: 35.

Preferably, the mannanase variant further comprises an amino acid substitution at one or more amino acid positions selected from the group consisting of X341F, X346T, X348S/R/N/M/G, X349T/S/G/D, X352N/G, X356Y/V/T/Q/H/C, and X379V according to the numbering of SEQ ID NO: 35.

Preferably, the mannanase variant further comprises an amino acid substitution at one or more amino acid positions selected from the group consisting of N341F, F346T, T348S/R/N/M/G, E349T/S/G/D, S352N/G, G356Y/V/T/Q/H/C, and D379V according to the numbering of SEQ ID NO: 35.

Preferably, the mannanase variant comprises an amino acid sequence which is at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to any of the amino acid sequences shown in SEQ ID NO: 35-37 or to a sequence according to positions 31-490 of SEQ ID NO: 35, preferably to a sequence according to positions 31-490 of SEQ ID NO: 35.

More preferably, the mannanase variant comprises an amino acid sequence which is at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35.

Most preferably, the mannanase variant comprises an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35.

Preferably, the mannanase of the enzyme composition is a mannanase variant of a parent mannanase, wherein the mannanase variant comprises an amino acid substitution at one or more amino acid positions selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and an amino acid sequence which is at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to any of the amino acid sequences shown in SEQ ID NO: 35-37 or to a sequence according to positions 31-490 of SEQ ID NO: 35, preferably to a sequence according to positions 31-490 of SEQ ID NO: 35.

Preferably, the mannanase of the enzyme composition is a mannanase variant of a parent mannanase, wherein the mannanase variant comprises an amino acid substitution at one or more amino acid positions selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35.

Preferably, the mannanase variant comprises one or more amino acid substitution selected from the group consisting of X86N, X89V/L, X96D, X101T/V, X103Y/E/A, X107N, X108G, X109Q/A, X112N, X119Y/H/T, X122S, X124E/C/D, X126E, X127A, X129M/L/F, X231Q/K/L/P/Y, X233V, X235H/R/L/Q/N/Y, X244I/V/N, X254W, X255Y/H/R, X264Q/V, X270T, X272I, X273T, X274E/C/Q, X281L, X286E/L/Q/A, X289F/M/H, X290A, X296H/F/Y, X301E/C/T, X309L, X312F/Y, X314P, X317T, and X319D/E according to the numbering of SEQ ID NO: 35 an amino acid sequence which is at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to any of the amino acid sequences shown in SEQ ID NO: 35-37 or to a sequence according to positions 31-490 of SEQ ID NO: 35, preferably to a sequence according to positions 31-490 of SEQ ID NO: 35.

Preferably, the mannanase variant comprises one or more amino acid substitution selected from the group consisting of X86N, X89V/L, X96D, X101T/V, X103Y/E/A, X107N, X108G, X109Q/A, X112N, X119Y/H/T, X122S, X124E/C/D, X126E, X127A, X129M/L/F, X231Q/K/L/P/Y, X233V, X235H/R/L/Q/N/Y, X244I/V/N, X254W, X255Y/H/R, X264Q/V, X270T, X272I, X273T, X274E/C/Q, X281L, X286E/L/Q/A, X289F/M/H, X290A, X296H/F/Y, X301E/C/T, X309L, X312F/Y, X314P, X317T, and X319D/E according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.

Preferably, the mannanase variant comprises one or more amino acid substitution selected from the group consisting of X86N, X89V/L, X96D, X101T/V, X103Y/E/A, X107N, X108G, X109Q/A, X112N, X119Y/H/T, X122S, X124E/C/D, X126E, X127A, X129M/L/F, X231Q/K/L/P/Y, X233V, X235H/R/L/Q/N/Y, X244I/V/N, X254W, X255Y/H/R, X264Q/V, X270T, X272I, X273T, X274E/C/Q, X281L, X286E/L/Q/A, X289F/M/H, X290A, X296H/F/Y, X301E/C/T, X309L, X312F/Y, X314P, X317T, and X319D/E according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.

Preferably, the mannanase variant comprises one or more amino acid substitution selected from the group consisting of X31V, X89V, X96D, X119Y/H/T, X264Q/V, X289F/M/H, X312F/Y, X348S/R/N/M/G, X349T/S/D/G, X352N/G, and X379V according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.

More preferably, the mannanase variant comprises at least one, at least two, at least three, at least four, at least five, at least six, at least seven, at least eight or all amino acid substitutions X89V, X96D, X119Y/H/T, X264Q/V, X289F/M/H, X312F/Y, X348S/R/N/M/G, X349T/S/D/G, and X352N/G according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.

More preferably, the mannanase variant comprises at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.

More preferably, the mannanase variant comprises the amino acid substitutions X89V, X96D, X119H, X264Q, X289M, and X312Y according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.

Most preferred is a mannanase variant comprising or consisting of the amino acid sequence according to positions 31-490 of SEQ ID NO: 35 with the amino acid substitution Q89V, N96D, A119H, E264Q, W289M, N312Y, T348N, E349G, and S352G according to the numbering of SEQ ID NO: 35.

In one embodiment the mannanase variant of the enzyme composition comprises
(i) an amino acid substitution at one or more amino acid positions corresponding to amino acid positions selected from the group consisting of 24, 123, and 256 according to the numbering of SEQ ID NO: 39 and an amino acid sequence which is at least 60%, but less than 100% identical to SEQ ID NO: 39, or
(ii) an amino acid substitution at one or more amino acid positions corresponding to amino acid positions selected from the group consisting of 10, 19, 30, 38, 59, 60, 67, 97, 103, 129, 143, 167, 168, 184, 225, 228, 235, 244, 258, 261, and 298 according to the numbering of SEQ ID NO: 38 and an amino acid sequence which is at least 60%, but less than 100% identical to SEQ ID NO: 38, or
(iii) an amino acid sequence which is at least 60% identical to SEQ ID NO: 40, or
(iv) an amino acid substitution at one or more amino acid positions corresponding to amino acid positions selected from the group consisting of 30, 81, 93, 95, 118, 138, 139, 200, 202, 210, 260, 283, 288, 294, and 295 according to the numbering of SEQ ID NO: 40 and an amino acid sequence which is at least 60%, but less than 100% identical to SEQ ID NO: 40.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 39 and comprises an amino acid substitution at one or more amino acid positions corresponding to amino acid positions selected from the group consisting of 24, 123, and 256 according to the numbering of SEQ ID NO: 39.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 39 and comprises one or more amino acid substitutions selected from the group consisting of X241, X1231, and X256G, preferably the combination of X241 and X256G according to the numbering of SEQ ID NO: 39.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 39 and comprises one or more amino acid substitutions selected from the group consisting of V241, V1231, and S256G, preferably the combination of V241 and S256G according to the numbering of SEQ ID NO: 39.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 38 and comprises an amino acid substitution at one or more amino acid positions corresponding to amino acid positions selected from the group consisting of 10, 19, 30, 38, 59, 60, 67, 97, 103, 129, 143, 167, 168, 184, 225, 228, 235, 244, 258, and 261, preferably in combination with the deletion at position 298*, according to the numbering of SEQ ID NO: 38.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 38 and comprises one or more amino acid substitutions selected from the group consisting of X10T, X19P, X30S, X38T, X59S, X60L, X67N, X97N, X103V, X129Y, X143Q, X167F, X168S, X184Q, X225P/G, X228T, X235Y, X244K, X258S, and X261N, preferably in combination with the deletion X298*, according to the numbering of SEQ ID NO: 38.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 38 and comprises one or more amino acid substitutions selected from the group consisting of N10T, E19P, T30S, E38T, V59S, Q60L, D67N, D97N, 1103V, M129Y, K143Q, Y167F, P168S, L184Q, C225P/G, V228T, L235Y, L244K, D258S, and R261N, preferably in combination with the deletion Q298*, according to the numbering of SEQ ID NO: 38.

In one embodiment, the mannanase variant has at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 40.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 40 and comprises an amino acid substitution at one or more amino acid positions corresponding to amino acid positions selected from the group consisting of 30, 81, 93, 95, 118, 138, 139, 200, 202, 210, 260, 283, 288, 294, and 295, preferably amino acid substitution at one or more amino acid positions corresponding to amino acid positions selected from the group consisting of 260, 288, 294, and 295, according to the numbering of SEQ ID NO: 40.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 40 and comprises one or more amino acid substitutions selected from the group consisting of X30T, X81P, X93Q, X95D, X118K, X138P, X139R, X200T, X202R, X210G, X260F, X283H, X2881, X294P, and X295V, preferably one or more amino acid substitutions selected from the group consisting of X260F, X2881, X294P, and X295V, according to the numbering of SEQ ID NO: 40.

In one embodiment, the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 40 and comprises one or more amino acid substitutions selected from the group consisting of A30T, L81P, Y93Q, S95D, D118K, A138P, D139R, N200T, S202R, R210G, W260F, N283H, L2881, L294P, and S295V, preferably one or more amino acid substitutions selected from the group consisting of W260F, L2881, L294P, and S295V, according to the numbering of SEQ ID NO: 40.

Mannanases with substitutions as described above are shown for instance in SEQ ID NO: 41.

Thus, in one embodiment the mannanase variant comprises an amino acid sequence which is at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 41.

### Cellulases

In one embodiment, the enzyme composition of the present invention comprises a cellulase. The cellulases and cellulase variants described herein have cellulase activity. “Cellulases” are enzymes capable of hydrolysing cellulose. Cellulases may be selected from cellobiohydrolases (1,4-β-D-glucan cellobiohydrolase, EC 3.2.1.91), endo-ss-1,4-glucanases (EC 3.2.1.4) and ssglucosidases (EC 3.2.1.21). Endoglucanases of EC class 3.2.1.4 may be named endoglucanase, endo-1,4-ss-D-glucan 4-glucano hydrolase, endo-1,4-beta-glucanase, carboxymethyl cellulase, and beta-1,4-glucanase.

Endoglucanases may be classified by amino acid sequence similarities (Henrissat, B. Accessed at UniProt Oct. 26, 2011) as family 5 that contains more than 20 endoglucanases of EC 3.2.1.4. Reference is also made to T.-M. Enveri, “Microbial Cellulases” in W. M. Fogarty, Microbial Enzymes and Biotechnology, Applied Science Publishers, p. 183-224 (1983); Methods in Enzymology, (1988) Vol. 160, p. 200-391 (edited by Wood, W. A. and Kellogg, S. T.); Beguin, P., “Molecular Biology of Cellulose Degradation”, Annu. Rev. Microbiol. (1990), Vol. 44, pp. 219248; Begun, P. and Aubert, J-P., “The biological degradation of cellulose”, FEMS Microbiology Reviews 13 (1994) p. 25-58; Henrissat, B., “Cellulases and their interaction with cellulose”, Cellulose (1994), Vol. 1, pp. 169-196.

The cellulase of the enzyme composition is selected from
a cellulase variant comprising an amino acid sequence which is at least 60% identical to SEQ ID NO: 42, and
a cellulase variant comprising an amino acid sequence which is at least 60% identical to SEQ ID NO: 46, SEQ ID NO: 43, SEQ ID NO: 45, SEQ ID NO: 44, or SEQ ID NO: 47.

Preferably, the cellulase variant of the enzyme composition is a cellulase comprising an amino acid sequence which is at least 60% identical to SEQ ID NO: 42.

Preferably, the cellulase variant has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to the amino acid sequence of SEQ ID NO: 42.

Preferably, the cellulase variant has at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to the amino acid sequence of SEQ ID NO: 42.

Preferably, the cellulase variant has at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to the amino acid sequence of SEQ ID NO: 42.

Most preferably the cellulase variant comprises or consists of SEQ ID NO: 42.

In one embodiment the cellulase of the enzyme composition comprises an amino acid sequence which is at least 60% identical to SEQ ID NO: 46, SEQ ID NO: 43, SEQ ID NO: 45, SEQ ID NO: 44, or SEQ ID NO: 47.

Preferably, the cellulase variant has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to any of the amino acid sequences shown SEQ ID NO: 43-47.

Preferably, the cellulase variant has at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to any of the amino acid sequences shown SEQ ID NO: 43-47.

Preferably, the cellulase variant has at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to any of the amino acid sequences shown in SEQ ID NO: 43-47.

In one embodiment, the cellulase according to the present invention preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 46.

In one embodiment, the cellulase variant has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 43.

In one embodiment, the cellulase variant has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 45.

In one embodiment, the cellulase variant has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with SEQ ID NO: 44.

In one embodiment, the cellulase variant has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity to any of the amino acid sequences shown in SEQ ID NO: 47.

Preferably, the composition comprises an amylase and one or more, preferably two or more enzymes selected from the group consisting of protease, mannanase, and cellulase, wherein
a. the amylase is an amylase variant of a parent amylase, wherein said amylase variant comprises
   (i) an amino acid substitution at position 25 according to the numbering of SEQ ID NO: 2,
   (ii) an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2,
   (iii) at least 80%, preferably at least 91% identity, but less than 100% sequence identity with any of SEQ ID NO: 1,
b. the protease is selected from
   I. a protease variant of a parent protease, wherein the protease variant comprises
      (i) an amino acid sequence comprising compared to SEQ ID NO: 16 at least two additional negative charges in the loop region of residues 98 to 104 according to the numbering of SEQ ID NO: 17, and
      (ii) an amino acid sequence which is at least 80%, but less than 100% identical to any of SEQ ID NO: 16, or
   II. a protease variant of a parent protease, wherein the protease variant comprises
      (i) an amino acid substitution at one or more amino acid positions corresponding to amino acid positions selected from the group consisting of 3, 4, 9, 19, 22, 43, 62, 63, 76, 87, 99, 101, 103, 104, 116, 118, 128, 129, 130, 156, 160, 167, 170, 182, 185, 188, 194, 205, 213, 217, 218, 232, 245, 256, 259, 262, and 271 according to the numbering of SEQ ID NO: 17 and an amino acid sequence which is at least 60%, but less than 100% sequence identity with SEQ ID NO: 22, or
      (ii) an amino acid substitution at one or more amino acid positions corresponding to amino acid positions selected from the group consisting of 24, 53, 76, 78, 101, 128, and 217 according to the numbering of SEQ ID NO: 17 and an amino acid sequence which is at least 80%, but less than 100% sequence identity with SEQ ID NO: 17, or
      (iii) an amino acid substitution at one or more amino acid positions corresponding to amino acid positions selected from the group consisting of 9, 89, 130, 133, 144, 159, 183, 189. 217, 224, 238, 243, 252, 259, 270 and 271 according to the numbering of SEQ ID NO: 19 and an amino acid sequence which is at least 60%, but less than 100% sequence identity with SEQ ID NO: 19, or
      (iv) an amino acid sequence which is at least 80% sequence identity with SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31,
c. the mannanase is selected from
   I. a mannanase variant of a parent mannanase, wherein the mannanase variant comprises
      (i) one or more amino acid substitution selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and
      (ii) an amino acid sequence which is at least 80%, but less than 100% identical to a sequence according to positions 31-490 of SEQ ID NO: 35, and
   II. a mannanase variant of a parent mannanase, wherein the mannanase variant comprises
      (i) an amino acid substitution at one or more amino acid positions corresponding to amino acid positions selected from the group consisting of 24, 123, and 256 according to the numbering of SEQ ID NO: 39 and an amino acid sequence which is at least 80%, but less than 100% identical to SEQ ID NO: 39, or
      (ii) an amino acid substitution at one or more amino acid positions corresponding to amino acid positions selected from the group consisting of 10, 19, 30, 38, 59, 60, 67, 97, 103, 129, 143, 167, 168, 184, 225, 228, 235, 244, 258, 261, and 298 according to the numbering of SEQ ID NO: 38 and an amino acid sequence which is at least 80%, but less than 100% identical to SEQ ID NO: 38, or
      (iii) an amino acid sequence which is at least 60% identical to SEQ ID NO: 40, or
      (iv) an amino acid substitution at one or more amino acid positions corresponding to amino acid positions selected from the group consisting of 30, 81, 93, 95, 118, 138, 139, 200, 202, 210, 260, 283, 288, 294, and 295 according to the numbering of SEQ ID NO: 40 and an amino acid sequence which is at least 80%, but less than 100% identical to SEQ ID NO: 40,
d. the cellulase is selected from
   I. a cellulase variant comprising an amino acid sequence which is at least 80% identical to SEQ ID NO: 42, or
   II. a cellulase variant comprising an amino acid sequence which is at least 80% identical to SEQ ID NO: 46, SEQ ID NO: 43, SEQ ID NO: 45, SEQ ID NO: 44, or SEQ ID NO: 47.

More preferably, the composition comprises an amylase and one or more, preferably two or more enzymes selected from the group consisting of protease, mannanase, and cellulase, wherein
a. the amylase is an amylase variant of a parent amylase, wherein said amylase variant comprises
   (i) an amino acid substitution at position 25 according to the numbering of SEQ ID NO: 2,
   (ii) an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2, and
   (iii) at least 80%, preferably at least 91% identity, but less than 100% sequence identity with any of SEQ ID NO: 1,
b. the protease a protease variant of a parent protease, wherein the protease variant comprises
   (i) an amino acid sequence comprising compared to SEQ ID NO: 16 at least two additional negative charges in the loop region of residues 98 to 104 according to the numbering of SEQ ID NO: 17, and
   (ii) an amino acid sequence which is at least 80%, but less than 100% identical to SEQ ID NO: 16,
c. the mannanase is a mannanase variant of a parent mannanase, wherein the mannanase variant comprises
   (i) one or more amino acid substitution selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and
   (ii) an amino acid sequence which is at least 80%, but less than 100% identical to a sequence according to positions 31-490 of SEQ ID NO: 35, and
d. the cellulase is a cellulase variant comprising an amino acid sequence which is at least 80% identical to SEQ ID NO: 42.

More preferably, the composition comprises an amylase and one or more, preferably two or more enzymes selected from the group consisting of protease, mannanase, and cellulase, wherein
a. the amylase is an amylase variant comprising
   (i) the amino acid substitution X4G according to the numbering of SEQ ID NO: 2,
   (ii) one or more, two or more, three or more, four or more, five or more, or all of the amino acid substitutions selected from the group consisting of X25H, X176K, X186E, X251E, X405M, and X482W according to the numbering of SEQ ID NO: 2,
   (iii) at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1, and
   (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2),
      or wherein the amylase is an amylase variant comprising
      (i) the amino acid substitution X25H according to the numbering of SEQ ID NO: 2,
      (ii) the amino acid substitution either X195F or X206Y according to the numbering of SEQ ID NO: 2,
      (iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
      (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
      (v) at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1,
b. the protease is a protease variant comprising
   (i) the amino acid substitution X101E according to the numbering of SEQ ID NO: 17, and
   (ii) an amino acid sequence which is at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 16,
c. the mannanase is a mannanase variant comprising
   (i) at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35, and
   (ii) an amino acid sequence which is at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35, and
d. the cellulase is a cellulase variant comprising an amino acid sequence which is at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 42.

More preferably, composition comprises an amylase and one or more, preferably two or more enzymes selected from the group consisting of protease, mannanase, and cellulase, wherein
a. the amylase variant comprises or consists of the amino acid sequence set forth in SEQ ID NO: 1 with the amino acid alterations X4Q+X25H+X176K+X186E+X251E+X405M+X482W, X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably X4Q+X25H+X176K+X186E+X251E+X405M+X482W according to the numbering of SEQ ID NO: 2,
b. the protease is a protease variant comprising or consisting of the amino acid sequence set forth in SEQ ID NO: 16 with the amino acid substitution R101E according to the numbering of SEQ ID NO: 17, i.e., only one amino acid exchange compared to SEQ ID NO: 16,
c. the mannanase is a mannanase variant comprising or consisting of the amino acid sequence according to positions 31-490 of SEQ ID NO: 35 with the amino acid substitution Q89V, N96D, A119H, E264Q, W289M, N312Y, T348N, E349G, and S352G according to the numbering of SEQ ID NO: 35,
d. the cellulase is a cellulase variant comprising or consisting of an amino acid sequence which is 100% identical to SEQ ID NO: 42.

Alternatively, the composition comprises a protease variant with the amino acid substitution X101E and the amino acid substitutions X3T, X4I, and X205I or the amino acid substitutions X156D and X262E according to the numbering of SEQ ID NO: 17, and an amino acid sequence which is at least 90%, but less than 100% identical to SEQ ID NO: 16.

Alternatively, the protease variant comprises an amino acid sequence which has at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 22 and one or more amino acid substitutions selected from the group consisting of X3T, X4I, X9D/E/R, X19L, X22R/A, X43R, X62D, X63D/E, X76D, X87N, X99A/D, X101M/R/E, X103A, X1041, X116L, X118V, X128L, X129Q, X130A, X156D/E, X160D/S, X167A, X170S, X182E/D, X185E/D, X188D, X194P, X205I, X213A, X217E/D/Q, X218D, X232V, X245R, X256D/E, X259D/E, X262D/E, and X271F according to the numbering of SEQ ID NO: 17, preferably the protease variant further comprises an insertion of D and E directly adjacent C-terminally of the position 99 according to the numbering of SEQ ID NO: 17, preferably, the protease variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to any of SEQ ID NO: 24.

Alternatively, the mannanase variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 40 and comprises one or more amino acid substitutions selected from the group consisting of X30T, X81P, X93Q, X95D, X118K, X138P, X139R, X200T, X202R, X210G, X260F, X283H, X2881, X294P, and X295V, preferably one or more amino acid substitutions selected from the group consisting of X260F, X2881, X294P, and X295V, according to the numbering of SEQ ID NO: 40, preferably the mannanase variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 41.

More preferably, the composition comprises an amylase and one or more, preferably two or more enzymes selected from the group consisting of protease, mannanase, and cellulase, wherein
a. the amylase is an amylase variant comprising
   (i) the amino acid substitution X4G according to the numbering of SEQ ID NO: 2,
   (ii) one or more, two or more, three or more, four or more, five or more, or all of the amino acid substitutions selected from the group consisting of X25H, X176K, X186E, X251E, X405M, and X482W according to the numbering of SEQ ID NO: 2,
   (iii) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1, and
   (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2),
   or wherein the amylase is an amylase variant comprising
   (i) the amino acid substitution X25H according to the numbering of SEQ ID NO: 2,
   (ii) the amino acid substitution either X195F or X206Y according to the numbering of SEQ ID NO: 2,
   (iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
   (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
   (v) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1,
b. the protease is a protease variant comprising
   (i) the amino acid substitution X101E according to the numbering of SEQ ID NO: 17, and
   (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 16,
      or the protease is a protease variant comprising the amino acid substitution X101E and the amino acid substitutions X3T, X4I, and X205I or the amino acid substitutions X156D and X262E according to the numbering of SEQ ID NO: 17, and an amino acid sequence which is at least 90%, but less than 100% identical to SEQ ID NO: 16,
      or the protease is a protease variant comprising an amino acid sequence which has at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 22 and one or more amino acid substitutions selected from the group consisting of X3T, X4I, X9D/E/R, X19L, X22R/A, X43R, X62D, X63D/E, X76D, X87N, X99A/D, X101M/R/E, X103A, X1041, X116L, X118V, X128L, X129Q, X130A, X156D/E, X160D/S, X167A, X170S, X182E/D, X185E/D, X188D, X194P, X205I, X213A, X217E/D/Q, X218D, X232V, X245R, X256D/E, X259D/E, X262D/E, and X271F according to the numbering of SEQ ID NO: 17, preferably the protease variant further comprises an insertion of D and E directly adjacent C-terminally of the position 99 according to the numbering of SEQ ID NO: 17, preferably, the protease variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to any of SEQ ID NO: 24,
c. the mannanase is a mannanase variant comprising
   (i) at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35, and
   (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35,
      or the mannanase is a mannanase variant comprising an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 40 and comprises one or more amino acid substitutions selected from the group consisting of X30T, X81P, X93Q, X95D, X118K, X138P, X139R, X200T, X202R, X210G, X260F, X283H, X2881, X294P, and X295V, preferably one or more amino acid substitutions selected from the group consisting of X260F, X2881, X294P, and X295V, according to the numbering of SEQ ID NO: 40, preferably the mannanase variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 41,
d. the cellulase is a cellulase variant comprising an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 42.

The composition may comprise an amylase and a protease and a mannanase as described herein, an amylase and a protease and a cellulase as described herein, an amylase and a mannanase and a cellulase herein, or an amylase and a protease, a mannanase, and a cellulase herein, preferably the composition comprises an amylase and a protease, a mannanase, and a cellulase as described herein.

Thus, more preferably, the composition comprises an amylase, a protease, a mannanase and a cellulase and
a. the amylase is an amylase variant comprising
   (i) the amino acid substitution X4G according to the numbering of SEQ ID NO: 2,
   (ii) one or more, two or more, three or more, four or more, five or more, or all of the amino acid substitutions selected from the group consisting of X25H, X176K, X186E, X251E, X405M, and X482W according to the numbering of SEQ ID NO: 2,
   (iii) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1, and
   (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2),
   or wherein the amylase is an amylase variant comprising
   (i) the amino acid substitution X25H according to the numbering of SEQ ID NO: 2,
   (ii) the amino acid substitution either X195F or X206Y according to the numbering of SEQ ID NO: 2,
   (iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
   (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
   (v) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1,
b. the protease is a protease variant comprising
   (i) the amino acid substitution X101E according to the numbering of SEQ ID NO: 17, and
   (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 16,
c. the mannanase is a mannanase variant comprising
   (i) at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35, and
   (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35, and
d. the cellulase is a cellulase variant comprising an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 42.

Most preferably, the composition comprises an amylase, a protease, a mannanase and a cellulase and
a. the amylase variant comprises or consists of the amino acid sequence set forth in SEQ ID NO: 1 with the amino acid alterations X4Q+X25H+X176K+X186E+X251E+X405M+X482W, X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably with X4Q+X25H+X176K+X186E+X251E+X405M+X482W according to the numbering of SEQ ID NO: 2,
b. the protease is a protease variant comprising or consisting of the amino acid sequence set forth in SEQ ID NO: 16 with the amino acid substitution R101E according to the numbering of SEQ ID NO: 17, i.e., only one amino acid exchange compared to SEQ ID NO: 16,
c. the mannanase is a mannanase variant comprising or consisting of the amino acid sequence according to positions 31-490 of SEQ ID NO: 35 with the amino acid substitution Q89V, N96D, A119H, E264Q, W289M, N312Y, T348N, E349G, and S352G according to the numbering of SEQ ID NO: 35, and
d. the cellulase is a cellulase variant comprising or consisting of an amino acid sequence which is 100% identical to SEQ ID NO: 42.

### Further Enzymes

In another embodiment, the composition comprising the enzyme composition as described herein further comprises one or more further enzymes different from the enzymes of the enzyme composition described herein. Preferably, the further enzyme is selected from the group consisting of, proteases, amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, beta-glucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the further enzyme is selected from the group consisting of lipases, hemicellulases, xylanases, DNases, dispersins, pectinases, oxidoreductases, cutinases, phosphodiesterases, licheninases, xanthan-lyases, and hexosaminidases, and combinations of at least two of the foregoing types. Most preferably, the further enzyme is a lipase.

### Lipases

“Lipases”, “lipolytic enzyme”, “lipid esterase”, all refer to an enzyme of EC class 3.1.1 (“carboxylic ester hydrolase”). Lipase means active protein having lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50). Lipases include those of bacterial or fungal origin.

In one aspect of the invention, a suitable lipase is selected from the following: lipases from Humicola (synonym Thermomyces), e.g. from H. lanuginosa (T. lanuginosus) as described in EP 258068, EP 305216, WO 92/05249 and WO 2009/109500 or from H. insolens as described in WO 96/13580; lipases derived from Rhizomucor miehei as described in WO 92/05249; lipase from strains of Pseudomonas (some of these now renamed to Burkholderia), e.g. from P. alcaligenes or P. pseudoalcaligenes (EP 218272, WO 94/25578, WO 95/30744, WO 95/35381, WO 96/00292), P. cepacia (EP 331376), P. stutzeri (GB 1372034), P. fluorescens, Pseudomonas sp. strain SD705 (WO 95/06720 and WO 96/27002), P. wisconsinensis (WO 96/12012), Pseudomonas mendocina (WO 95/14783), P. glumae (WO 95/35381, WO 96/00292); lipase from Streptomyces griseus (WO 2011/150157) and S. pristinaespiralis (WO 2012/137147), GDSL-type Streptomyces lipases (WO 2010/065455); lipase from Thermobifida fusca as disclosed in WO 2011/084412; lipase from Geobacillus stearothermophilus as disclosed in WO 2011/084417; Bacillus lipases, e.g. as disclosed in WO 00/60063, lipases from B. subtilis as disclosed in Dartois et al. (1992), Biochemica et Biophysica Acta, 1131, 253-360 or WO 2011/084599, B. stearothermophilus (JP S64-074992) or B. pumilus (WO 91/16422); lipase from Candida antarctica as disclosed in WO 94/01541; cutinase from Pseudomonas mendocina (U.S. Pat. No. 5,389,536, WO 88/09367); cutinase from Magnaporthe grisea (WO 2010/107560); cutinase from Fusarium solani pisi as disclosed in WO 90/09446, WO 00/34450 and WO 01/92502; and cutinase from Humicola lanuginosa as disclosed in WO 00/34450 and WO 01/92502.

Such suitable lipase variants are e.g. those which are developed by methods as disclosed in WO 95/22615, WO 97/04079, WO 97/07202, WO 00/60063, WO 2007/087508, EP 407225 and EP 260105.

Commercially available lipase enzymes include but are not limited to those sold under the trade names Lipolase™, Lipex™, Lipolex™ and Lipoclean™ (Novozymes A/S), Lumafast (originally from Genencor), Preferenz L (DuPont), Biotouch LL100 (AB Enzymes), and Lipomax (GistBrocades/now DSM).

In one embodiment, lipase is selected from fungal triacylglycerol lipase (EC class 3.1.1.3). Fungal triacylglycerol lipase may be selected from lipases of Thermomyces lanuginosus. In one embodiment, the Thermomyces lanuginosa lipase is selected from triacylglycerol lipase according to amino acids 1-269 of SEQ ID NO: 2 of U.S. Pat. No. 5,869,438 and variants thereof having lipolytic activity.

Thermomyces lanuginosa lipase may be selected from variants having lipolytic activity which are at least 90%, at least 91%, at least 92%, at least 93%, at least 94%, at least 95%, at least 96%, at least 97%, at least 98%, at least 99%, or 100% identical when compared to the full length polypeptide sequence of amino acids 1-269 of SEQ ID NO: 2 of U.S. Pat. No. 5,869,438.

Thermomyces lanuginosa lipase may be selected from variants having lipolytic activity comprising conservative mutations only, which do not pertain the functional domain of amino acids 1-269 of SEQ ID NO: 2 of U.S. Pat. No. 5,869,438. Lipase variants of this embodiment having lipolytic activity may be at least 95%, at least 96%, at least 97%, at least 98%, or at least 99% similar when compared to the full-length polypeptide sequence of amino acids 1-269 of SEQ ID NO: 2 of U.S. Pat. No. 5,869,438.

Thermomyces lanuginosa lipase may be selected from variants having lipolytic activity comprising the following amino acid substitutions when compared to amino acids 1-269 of SEQ ID NO: 2 of U.S. Pat. No. 5,869,438: T231R and N233R. Said lipase variants may further comprise one or more of the following amino acid exchanges when compared to amino acids 1-269 of SEQ ID NO: 2 of U.S. Pat. No. 5,869,438: Q4V, V60S, A150G, L227G, P256K.

Thermomyces lanuginosa lipase may be selected from variants having lipolytic activity comprising the amino acid substitutions T231R, N233R, Q4V, V60S, A150G, L227G, P256K within the polypeptide sequence of amino acids 1-269 of SEQ ID NO: 2 of U.S. Pat. No. 5,869,438 and are at least 95%, at least 96%, or at least 97% similar when compared to the full length polypeptide sequence of amino acids 1-269 of SEQ ID NO: 2 of U.S. Pat. No. 5,869,438.

Thermomyces lanuginosa lipase may be selected from variants having lipolytic activity comprising the amino acid substitutions T231R and N233R within amino acids 1-269 of SEQ ID NO: 2 of U.S. Pat. No. 5,869,438 and are at least 95%, at least 96%, at least 97%, at least 98%, or at least 99% similar when compared to the full length polypeptide sequence of amino acids 1-269 of SEQ ID NO: 2 of U.S. Pat. No. 5,869,438.

Thermomyces lanuginosa lipase may be a variant of amino acids 1-269 of SEQ ID NO: 2 of U.S. Pat. No. 5,869,438 having lipolytic activity, wherein the variant of amino acids 1-269 of SEQ ID NO: 2 of U.S. Pat. No. 5,869,438 is characterized in containing the amino acid substitutions T231R and N233R.

Thermomyces lanuginosa lipase may be selected from variants having lipolytic activity preferably comprising at least one, preferably more than one, more preferably all of the following substitutions N11K, A18K, G23K, K24A, V771, D130A, V1541, V187T, T189Q within the polypeptide sequence of amino acids 1-269 of SEQ ID NO: 1 of WO2015/010009 and are at least 95%, at least 96%, or at least 97% similar when compared to the full length polypeptide sequence of amino acids 1-269 of SEQ ID NO: 1 of WO2015/010009.

### Methods of Obtaining the Enzyme and Making the Enzyme Composition

Another embodiment of the present invention is a method of making the enzyme composition comprising a protease, a mannanase and/or a cellulase as described herein, preferably comprising the steps of
a) obtaining a sequence of a variant of a protease, a mannanase and/or a cellulase as described herein,
b) producing said variant of a protease, a mannanase and/or a cellulase using the sequence obtained under a), as described herein, and
c) mixing at least two or more enzymes selected from the group consisting of protease, mannanase, and cellulase as described herein in one or more steps.

Ways of obtaining a specific enzyme variant are known to the skilled person. Ways of introducing amino acid alterations as described, e.g., a substitution or a deletion, into protein sequences are well known in the art. The variants of the enzymes as described herein may be prepared using any mutagenesis procedure known in the art, such as site-directed mutagenesis, synthetic gene construction, semi-synthetic gene construction, random mutagenesis, shuffling, etc. Site-directed mutagenesis is a technique in which one or more (several) mutations are created at one or more defined sites in a polynucleotide. Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the nucleotide to be mutated and subsequent ligation of an oligonucleotide containing the mutation in the polynucleotide. Usually, the restriction enzyme that digests the plasmid and the oligonucleotide is the same, permitting sticky ends of the plasmid and insert to ligate to one another. See, e.g., Scherer and Davis, 1979, Proc. Natl. Acad. Sci. USA 76: 4949-4955; and Barton et al., 1990, Nucleic Acids Res. 18: 7349-4966. Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16. Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semisynthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

The obtained enzyme can be produced in an industrial scale and subsequently purified. Industrial production of enzymes usually is done by cultivating a host cell (also called fermentation) which expresses the enzyme. Suitable host cells are described herein. A nucleic acid sequence encoding the respective enzyme described herein can be transformed into the host cell, which is subsequently cultivated under conditions suitable for the host cell to produce the enzyme. In a preferred embodiment, the enzyme is purified from the host cell.

Producing an enzyme, preferably comprises the steps of
(a) providing a host cell comprising a heterologous nucleic acid construct comprising a polynucleotide encoding the enzyme described herein by introducing the nucleic acid construct comprising the polynucleotide encoding the enzyme as described herein into the host cell;
(b) cultivating the recombinant host cell of step (a) under conditions conductive for the expression of the polynucleotide; and
(c) optionally, recovering the enzyme encoded by the polynucleotide.

Cultivation of the host cell normally takes place in a suitable nutrient medium allowing the recombinant cells to grow and express the desired protein. At the end of fermentation, the fermentation broth is collected and may be further processed, wherein the fermentation broth comprises a liquid fraction and a solid fraction. The enzyme of interest may be further purified from the fermentation broth.

The amylase, protease, mannanase and cellulase as described herein may be secreted (into the liquid fraction of the fermentation broth) or may not be secreted from the microbial cells (and therefore is comprised in the cells of the fermentation broth). Depending on this, the enzyme may be recovered from the liquid fraction of the fermentation broth or from cell lysates. Preferably, the enzyme is secreted from the cell into the fermentation broth, preferably by means of a secretion signal peptide added to the terminus of the amino acid sequence of the respective enzyme. Recovery of the enzyme can be achieved by methods known to those skilled in the art. Suitable methods for recovery of proteins from fermentation broth include but are not limited to collection, centrifugation, filtration, extraction, and precipitation. If the product of interest precipitates or crystallizes in the fermentation broth or binds at least in part to the particulate matter of the fermentation broth additional treatment steps might be needed to release the protein of interest from the biomass or to solubilize protein of interest crystals and precipitates. WO0043502A1, WO2008110498 A1, and WO2017097869A1 describe a method for recovering a protein of interest, which precipitates and/or crystallizes during fermentation, from the fermentation broth. In case the desired protein is comprised in the cells of the fermentation broth release of the product of interest from the cells might be needed. Release from the cells can be achieved for instance, but not being limited thereto, by cell lysis with techniques well known to the skilled person, e.g., lysozyme treatment, ultrasonic treatment, French press or combinations thereof.

The amylase, protease, mannanase and cellulase as described herein may be purified from the fermentation broth by methods known in the art. For example, the respective enzyme may be isolated from the fermentation broth by conventional procedures including, but not limited to, centrifugation, filtration, extraction, spray-drying, evaporation, or precipitation. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). The purified polypeptide may then be concentrated by procedures known in the art including, but not limited to, ultrafiltration and evaporation, in particular, thin film evaporation.

### Preparations Comprising Enzyme Composition

The enzyme composition described herein may be further processed to form an enzyme composition containing preparation. Hence, also claimed herein is a preparation comprising the enzyme composition described herein and at least one additional component.

Thus, the present invention therefore also refers to a method of making a preparation comprising the steps of mixing
a) an enzyme composition as described herein; and
b) one or more components described herein.

The preparation can be a non-complex formulation, e.g., an enzyme composition containing formulation, or a complex formulation, e.g., a detergent composition.

In one embodiment of the present invention, the enzyme composition is formulated as an enzyme composition containing formulation, preferably a concentrated enzyme composition containing formulation. The enzyme composition containing formulation can be either solid or liquid. Protein formulations can be obtained by using techniques known in the art. For instance, without being limited thereto, solid enzyme formulations can be obtained by extrusion or granulation. Suitable extrusion and granulation techniques are known in the art and are described for instance in WO 94/19444 A1 and WO 97/43482 A1.

Preferably, the enzyme formulation comprises the enzymes of the present invention in an amount of 2-120 mg active enzymes per g of enzyme formulation, preferably 6-80 mg/g, 6-60 mg/g, or 10-40 mg/g.

In one embodiment, the enzyme composition containing formulation, in particular the liquid enzyme formulation, comprises in addition one or more additional compounds selected from the group consisting of solvent, salt, pH regulator, preservative, enzyme stabilizer, and thickening agent. Preferably, the enzyme composition containing formulation is devoid of surfactants. The solvent may be water and/or an organic solvent. Aqueous enzyme composition containing formulations of the invention may comprise water in amounts of more than about 50% by weight, more than about 60% by weight, more than about 70% by weight, or more than about 80% by weight, all relative to the total weight of the enzyme formulation. The enzyme composition containing formulations of the invention may comprise an organic solvent in amounts of more than 30%, more than 40%, more than about 50% by weight, more than about 60% by weight, more than about 70% by weight, or more than about 80% by weight, all relative to the total weight of the enzyme formulation. The organic solvent may be a water-miscible solvent. The organic solvent may be one or more selected from the group consisting of glycerol, propanediol, polypropylene glycol, and polyethylene glycol.

In one embodiment, the enzyme composition containing formulation comprises at least one preservative. Preferably, preservative means substances that are added to a liquid composition for the purpose of preservation, meaning more preferably that compounds known to have preserving features comprised in a liquid composition formed in the production process are excluded from the term preservatives. In one embodiment, the preservative is selected from the group consisting of 2-phenoxyethanol, glutaraldehyde, 2-bromo-2-nitropropane-1,3-diol, and formic acid in acid form or as its salt, and 4,4′-dichloro 2-hydroxydiphenylether. Usually, the liquid compositions of the invention comprise at least one preservative in amounts below 10 ppm, such as in amounts ranging from 2 ppm to 5% by weight relative to the total weight of the liquid composition. Alternatively, the enzyme composition containing formulation is free from preservatives, meaning that preservatives are comprised in amounts less than 1 ppm, preferably 0 ppm. Preferably, the enzyme composition containing formulation comprises respective enzyme stabilizing systems. Preferably, the enzyme composition containing formulation described herein comprises from about 0.001% to about 10%, from about 0.005% to about 8%, or from about 0.01% to about 6%, by weight of the composition, of an enzyme stabilizing system. The enzyme stabilizing system can be any stabilizing system which is compatible with the enzymes of the enzyme composition. Preferably, the enzyme stabilizing system comprises at least one compound selected from the group consisting of polyols (preferably, 1,3-propanediol, ethylene glycol, glycerol, 1,2-propanediol, or sorbitol), inorganic salts (preferably, CaCl2), MgCl2, or NaCl), short chain (preferably, C₁-C₃) carboxylic acids or salts thereof (preferably, formic acid, formate (preferably, sodium formate), acetic acid, acetate, or lactate), borate, boric acid, boronic acids (preferably, 4-formyl phenylboronic acid (4-FPBA)), peptide aldehydes (preferably, Z-VAL-H or Z-GAY-H), peptide acetals, and peptide aldehyde hydrosulfite adducts, depending on the enzymes contained in the respective enzyme composition. Preferably, in case a protease is present the enzyme stabilizing system comprises a combination of at least two of the compounds selected from the group consisting of salts, polyols, and short chain carboxylic acids and preferably one or more of the compounds selected from the group consisting of borate, boric acid, boronic acids (preferably, 4-formyl phenylboronic acid (4-FPBA)), peptide aldehydes, peptide acetals, and peptide aldehyde hydrosulfite adducts. In case a protease is contained in the enzyme composition, it is preferred that the stabilizing system comprises a protease inhibitor, preferably selected from borate, boric acid, boronic acids (preferably, 4-FPBA), peptide aldehydes (preferably, peptide aldehydes like Z-VAL-H or Z-GAY-H), peptide acetals, and peptide aldehyde hydrosulfite adducts, preferably the protease inhibitor is a peptide aldehyde, preferably Z-VAL-H or Z-GAY-H. In one embodiment, the stabilizing system does not comprise a protease inhibitor. Preferably, the enzyme formulation is boron-free. In case an amylase and/or a protease is present the formulation may comprise a calcium salt, preferably calcium chloride.

Preferably, the liquid formulation containing the enzyme composition comprises or consists of the enzyme composition, a solvent, at least one enzyme stabilizing system, and optionally a preservative and optionally a further enzyme different from the enzymes of the enzyme composition described herein. Preferably, the amylase variant formulation is devoid of surfactants.

Thus, the present invention therefore also refers to a method for making an enzyme composition containing formulation, preferably a concentrated enzyme composition containing formulation, comprising the steps of mixing
a) an enzyme composition as described herein; and
b) one or more components selected from the group consisting of solvent, enzyme stabilizing system, preservative, and a further enzyme different from the enzymes of the enzyme composition described herein.

### Detergent Compositions

In one embodiment, the present invention is directed to the use of the enzyme composition in a detergent composition. Thus, the present invention is also directed to a detergent composition comprising the enzyme composition described herein and one or more detergent component. Thus, the present invention therefore also refers to a method for making a detergent composition comprising the steps of mixing
a) an enzyme composition as described herein; and
b) one or more detergent component described herein.

Further, the present invention therefore also refers to a method for making a detergent composition with improved amylase stability and/or for providing a detergent composition with improved wash performance comprising the steps of mixing
a) an enzyme composition as described herein; and
b) one or more detergent component described herein.

The one or more detergent component may be selected from the group consisting of additional enzymes different from the enzymes of the enzyme composition described herein, enzyme stabilizing system, surfactant, defoamer, builder, polymer, bleaching system (bleach), rheology modifier, hydrotrope, softening agent, desiccant, whitening agent, buffer, preservative, anti-corrosion additive, dyestuff and fragrance.

Preferably, at least one component of the detergent is selected from the group consisting of surfactant, builder, polymer, preservative, and further enzyme different from the enzymes of the enzyme composition described herein.

Preferably one or more of the detergent component, preferably the surfactant and/or the builder, is bio-degradable and/or bio-based.

Detergent components may have more than one function in the final application of a detergent composition, therefore any detergent component mentioned in the context of a specific function herein, may also have another function in the final application of a detergent composition. The function of a specific detergent component in the final application of a detergent composition usually depends on its amount within the detergent composition, i.e., the effective amount of a detergent component. Detergent components vary in type and/or amount in a detergent composition depending on the desired application such as laundering white textiles, colored textiles, and wool. The component(s) chosen further depends on physical form of a detergent composition (liquid, solid, gel, provided in pouches or as a tablet, etc.). The component(s) chosen e.g. for laundering formulations further depend on regional conventions which themselves are related to aspects like washing temperatures used, mechanics of laundry machine (vertical vs. horizontal axis machines), water consumption per wash cycle etc. and geographical characteristics like average hardness of water.

In one embodiment, a detergent composition is a formulation of more than two detergent components, wherein at least one component is effective in stain-removal, at least one component is effective in providing the optimal cleaning conditions, and at least one component is effective in maintaining the physical characteristics of the detergent.

The detergent composition can be a liquid or solid detergent composition or a combination of liquid and solid detergent composition. The liquid detergent composition is preferably a gel detergent composition. The solid detergent composition can be a soap bar or a powder detergent composition, preferably a powder detergent composition, wherein the powder detergent composition can be pressed to a tablet.

The detergent composition can be a single unit dose or multi unit dose composition. The detergent composition can be in the form of a pouch, including multi-compartment pouches. The detergent composition can be a laundry or dish washing detergent composition, suitable for home care and/or industrial and institutional (I&I) cleaning. Both laundry and dish wash composition can be in the form of a hand wash or automated wash composition. Preferably the dish wash composition is an Automatic Dish Wash (ADW).

Detergent pouches can be of any form, shape and material which is suitable for holding the composition, e.g., without allowing the release of the composition from the pouch prior to water contact. The pouch is made from water-soluble film, which encloses an inner volume. Said inner volume can be divided into compartments of the pouch. Preferred films are polymeric materials preferably polymers which are formed into a film or sheet, e.g., polyvinyl alcohol copolymers and hydroxypropyl methyl cellulose (HPMC). The pouches can comprise a solid laundry detergent composition or part components and/or a liquid detergent composition or part components separated by the water-soluble film. The compartment for liquid components can be different in composition from compartments containing solids (see e.g. US 2009/0011970).

Preferably, the enzyme composition according to the present invention may be added to a detergent composition in an amount corresponding to 0.002 to 6 mg of active enzyme variant per g of detergent composition, preferably, 0.005-5 mg/g, 0.005-3 mg/g, 0.01-2 mg/g, or 0.05-2 mg/g. In one embodiment, the detergent composition has a pH in the range of 5-12, preferably in the range of 6-11, more preferably in a range selected from 6-10, 7-9, and 7.5-8.5. In one embodiment, the formulation is a detergent composition, preferably a liquid detergent composition.

In one embodiment, the detergent compositions according to the invention comprise one or more surfactant(s). According to its ionic charge, a surfactant is called non-ionic, anionic, cationic, or amphoteric.

The detergent composition of the present invention may comprise one or more surfactants, which may be anionic and/or cationic and/or non-ionic and/or semi-polar and/or zwitterionic, or a mixture thereof. In preferred embodiments, the detergent compositions of the invention comprise at least one surfactant. In a particular embodiment, the detergent composition of the present invention includes a mixture of one or more nonionic surfactants and one or more anionic surfactants. The surfactant(s) is/are typically present at a level of from about 0.1 to 60 wt.-%, such as 1 to 40 wt.-%, 3 to 20 wt.-% or 3 to 10 wt.-%. The surfactant(s) is/are chosen based on the desired cleaning application, and includes any conventional surfactant(s) known in the art. Any surfactant known in the art for use in detergents may be utilized. Non-limiting examples of surfactants are disclosed McCutcheon's 2016 Detergents and Emulsifiers, and McCutcheon's 2016 Functional Materials, both North American and International Edition, MC Publishing Co, 2016 edition. Further useful examples are disclosed in earlier editions of the same publications which are known to those skilled in the art.

When included therein, the detergent will usually comprise from about 1 to 40 wt.-%, such as 5 to 30 wt.-%, 5 to 15 wt.-% or 20 to 25 wt.-%, of an anionic surfactant. Non-limiting examples of anionic surfactants include sulfates and sulfonates, in particular linear alkyl benzene sulfonates (LAS), isomers of LAS, branched alkyl benzene sulfonates (BABS), phenyl alkane sulfonates, alpha-olefin sulfonates (AOS), olefin sulfonates, alkene sulfonates, alkane-2,3-diylbis(sulfates), hydroxy alkane sulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxy sulfates or fatty alcohol ether sulfates), secondary alkane sulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenyl succinic acid, dodecenyl/tetradecenyl succinic acid (DTSA), fatty acid derivatives of amino acids, diesters and monoesters of sulfo succinic acid or soap, and combinations thereof.

When included therein, the detergent will usually comprise from about 0 to 10 wt.-% of a cationic surfactant. Non-limiting examples of cationic surfactants include alkyl dimethyl ethanolamine quat (ADMEAQ), cetyl trimethyl ammonium bromide (CTAB), dimethyl distearyl ammonium chloride (DSDMAC), and alkyl benzyl dimethyl ammonium, alkyl quaternary ammonium compounds, alkoxylated quaternary ammonium (AQA) compounds, and combinations thereof. When included therein, the detergent will usually comprise from about 0.2 to 40 wt.-% of a nonionic surfactant, e.g. 0.5 to 30 wt.-%, in particular 1 to 20 wt.-%, 3 to 10 wt.-%, 3 to 5 wt.-% or 8 to 12 wt.-%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkyl phenol ethoxylates (APE), nonyl phenol ethoxylates (NPE), alkyl polyglycosides (APG), alkoxylated amines, fatty acid monoethanolamides (FAM), fatty acid diethanol amides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually comprise from about 0 to 10 wt.-% of a semipolar surfactant. Non-limiting examples of semipolar surfactants include amine oxides (AO) such as alkyl dimethyl amine oxide, N-(coco alkyl)-N,N-dimethyl amine oxide and N-(tallow-alkyl)-N,N-bis-(2-hydroxy ethyl) amine oxide, fatty acid alkanol amides and ethoxylated fatty acid alkanol amides, and combinations thereof.

When included therein, the detergent will usually comprise from about 0 to 10 wt.-% of a zwitterionic surfactant. Non-limiting examples of zwitterionic surfactants include betaine, alkyl dimethyl betaine, sulfo betaine, and combinations thereof.

The detergent compositions according to the invention may comprise one or more compounds selected from complexing agents (chelating agents (chelants), sequestrating agents), precipitating agents, and ion exchange compounds which may form water-soluble complexes with calcium and magnesium. Such compounds may be called “builders” or “building agents” herein, without meaning to limit such compounds to this function in the final application of a detergent composition.

In one embodiment, the detergent composition of the invention comprises at least one builder selected from non-phosphate based builders such as sodium gluconate, citrate(s), silicate(s), carbonate(s), phosphonate(s), amino carboxylate(s), polycarboxylate(s), polysulfonate(s), and polyphosphonate(s). In one embodiment, the detergent composition of the invention comprises a strong sequestering builder. Preferably, detergent compositions of the current invention are free from phosphate, meaning essentially free from phosphate-based builders. Herein, “essentially free from phosphate” is to be understood as meaning that the content of phosphate and polyphosphate is in sum in the range of 10 ppm to 1% by weight, determined by gravimetry and referring to the respective inventive detergent composition. In another preferred embodiment, the detergent composition comprises phosphonate, wherein the phosphonate is preferably DTPMP and/or HEDP.

In one embodiment, the detergent compositions of the invention comprise at least one “citrate” selected from the mono- and the dialkali metal salts and in particular the mono- and preferably the trisodium salt of citric acid, ammonium or substituted ammonium salts of citric acid as well as citric acid as such. Citrate can be used as the anhydrous compound or as the hydrate, for example as sodium citrate dihydrate. The citrate may be comprised in a total amount in the range of 0% to about 20% by weight, in the range of about 0.5% to about 10% by weight, or in the range of 1-5% by weight, all relative to the total weight of the detergent composition. In one embodiment, the detergent composition of the invention comprises a total amount of citrate in the range of about 1-3% relative to the total weight of the detergent composition.

Detergent compositions of the invention may comprise one or more silicates. “Silicate(s)” in the context of the present invention include in particular sodium disilicate and sodium metasilicate, aluminosilicates such as sodium aluminosilicates like zeolith A (i.e. Na₁₂(AlO₂)₁₂(SiO₂)₁₂*27H₂O), and sheet silicates, in particular those of the formula alpha-Na₂Si₂O₅, beta-Na₂Si₂O₅, and delta-Na₂Si₂O₅.

Detergent compositions of the invention may comprise one or more carbonates. The term “carbonate(s)” includes alkali metal carbonates and alkali metal hydrogen carbonates, preferred are the sodium salts. Particularly suitable is sodium carbonate (Na₂CO₃).

Detergent compositions of the invention may comprise one or more phosphonates. “Phosphonates” include, but are not limited to 2-phosphinobutane-1,2,4-tricarboxylic acid (PBTC); ethylenediaminetetra(methylenephosphonic acid) (EDTMPA); 1-hydroxyethane-1,1-diphosphonic acid (HEDP), CH₂C(OH)[PO(OH)₂]₂; aminotris(methylenephosphonic acid) (ATMP), N[CH₂PO(OH)₂]₃; aminotris(methylenephosphonate), sodium salt (ATMP), N[CH₂PO(ONa)₂]₃; 2-hydroxyethyliminobis(methylenephosphonic acid), HOCH₂CH₂N[CH₂PO(OH)₂]₂; diethylenetriaminepenta(methylenephosphonic acid) (DTPMP), (HO)₂POCH₂N[CH₂CH₂N[CH₂PO(OH)₂]₂]₂; diethylenetriaminepenta(methylenephosphonate), sodium salt, C₉H₍₂₃₋ₓ₎N₃NaₓO₁₅P₅ (x=7); hexamethylenediamine(tetramethylenephosphonate), potassium salt, C₁₀H₍₂₈₋ₓ₎N₂KₓO₁₂P₄ (x=6); and bis(hexamethylene)triamine(pentamethylenephosphonic acid), (HO₂)POCH₂N[(CH₂)₂N[CH₂PO(OH)₂]₂]₂. Salts thereof may be suitable, too.

Detergent compositions of the invention may comprise one or more aminocarboxylates. Nonlimiting examples of suitable “amino carboxylates” include, but are not limited to: diethanol glycine (DEG), dimethylglycine (DMG), nitrilitriacetic acid (NTA), N-hydroxyethylaminodiacetic acid, ethylenediaminetetraacetic acid (EDTA), N-(2hydroxyethyl)iminodiacetic acid (HEIDA), hydroxyethylenediaminetriacetic acid, N-hydroxyethyl-ethylenediaminetriacetic acid (HEDTA), hydroxyethylenediaminetetraacetic acid, diethylenetriaminepentaacetic acid (DTPA), and methylglycinediacetic acid (MGDA), glutamic acid-diacetic acid (GLDA), iminodisuccinic acid (IDS), hydroxyiminodisuccinic acid, ethylenediaminedisuccinic acid (EDDS), aspartic acid-diacetic acid, and alkali metal salts or ammonium salts thereof. Further suitable are aspartic acid-N-monoacetic acid (ASMA), aspartic acid-N,N-diacetic acid (ASDA), aspartic acid-N-monopropionic acid (ASMP), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), alpha-alanine-N,N-diacetic acid (alpha-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts or ammonium salts thereof. Preferred are MGDA or EDDS. The term “ammonium salts” as used in in this context refers to salts with at least one cation that bears a nitrogen atom that is permanently or temporarily quaternized. Examples of cations that bear at least one nitrogen atom that is permanently quaternized include tetramethylammonium, tetraethylammonium, dimethyldiethyl ammonium, and n-C₁₀-C₂₀-alkyl trimethyl ammonium. Examples of cations that bear at least one nitrogen atom that is temporarily quaternized include protonated amines and ammonia, such as monomethyl ammonium, dimethyl ammonium, trimethyl ammonium, monoethyl ammonium, diethyl ammonium, triethyl ammonium, n-C₁₀-C₂₀-alkyl dimethyl ammonium 2-hydroxyethylammonium, bis(2-hydroxyethyl) ammonium, tris(2-hydroxyethyl)ammonium, N-methyl 2-hydroxyethyl ammonium, N,N-dimethyl-2-hydroxyethylammonium, and especially NH₄.

In one embodiment, detergent compositions of the invention comprise more than one builder. Preferably, inventive detergent compositions contain less than 0.2% by weight of nitrilotriacetic acid (NTA), or 0.01 to 0.1% NTA by weight relative to the total weight of the detergent composition.

In one embodiment, the detergent composition of the invention comprise at least one aminocarboxylate selected from methylglycine diacetate (MGDA), glutamic acid diacetate (GLDA), and the respective salts thereof, e.g., alkali (such as sodium) salts thereof in amounts in the range of 0.1% to 25.0% by weight, in the range of 1.0% to 18.0% by weight, in the range of 3.0% to 15.0% by weight, in the range of 3.0% to 10.0% by weight, or in the range of 5.0% to 8.0% by weight relative to the total weight of the detergent composition.

The detergent compositions of the invention may comprise one or more hydrotropes. One or more hydrotropes may be selected from organic solvents such as ethanol, isopropanol, ethylene glycol, 1,2-propylene glycol, and further organic solvents known in the art that are water-miscible under normal conditions without limitation. In one embodiment, the detergent composition of the invention comprises 1,2-propylene glycol in a total amount in the range of 5-10% by weight, preferably of about 6% by weight, all relative to the total weight of the detergent composition. Further non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycol ethers, sodium hydroxy naphthoate, sodium hydroxy naphthalene sulfonate, sodium ethyl hexyl sulfate, and combinations thereof. In one embodiment, the detergent composition comprises at least one preservative. Preferably, preservative means substances that are added to a liquid composition for the purpose of preservation, meaning more preferably that compounds known to have preserving features comprised in a liquid composition formed in the production process are excluded from the term preservatives. In one embodiment, the preservative is selected from the group consisting of 2-phenoxyethanol, glutaraldehyde, 2-bromo-2-nitropropane-1,3-diol, and formic acid in acid form or as its salt, and 4,4′-dichloro 2-hydroxydiphenylether. Usually, the liquid compositions of the invention comprise at least one preservative in amounts below 10 ppm, such as in amounts ranging from 2 ppm to 5% by weight relative to the total weight of the liquid composition. Alternatively, the detergent composition is free from preservatives, meaning that preservatives are comprised in amounts less than 1 ppm, preferably 0 ppm.

In one embodiment, the detergent composition comprising an enzyme composition as described herein further comprises one or more further enzyme different from the enzymes of the enzyme composition described herein. Preferably, the further enzyme is selected from the group consisting of, proteases, amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, beta-glucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the further enzyme is selected from the group consisting of lipases, hemicellulases, xylanases, DNases, dispersins, pectinases, oxidoreductases, cutinases, phosphodiesterases, licheninases, xanthan-lyases, and hexosaminidases, and combinations of at least two of the foregoing types. Most preferably, the further enzyme is a lipase.

Particular preferred additional enzymes are disclosed elsewhere herein and that description is incorporated by reference also to this part of the description.

The detergent compositions may comprise water-soluble sources of calcium and/or magnesium ions. In one embodiment, at the detergent composition comprises an enzyme stabilizing system as described herein. Preferably, in particular in the case of liquid detergent compositions and in case a protease is present, the detergent composition may comprise at least one protease inhibitor as described herein, preferably selected from boronic acid derivatives, preferably 4-FPBA, and peptide aldehyde, preferably Z-VAL-H or Z-GAY-H. Preferably, the detergent composition is boron-free.

In one embodiment, the invention relates to a method to provide a detergent composition, preferably a liquid detergent composition, more preferably a liquid laundering detergent composition, comprising the steps of mixing in one or more steps
(a) at least one enzyme composition according to the invention, preferably wherein the enzymes of the enzyme composition are provided with an enzyme formulation as described herein; and
(b) at least one detergent component, preferably selected from surfactant, builder, polymer, preservative, and an enzyme different from the enzymes of the enzyme composition described herein, present in amounts effective in cleaning performance and/or effective in maintaining the physical characteristics of the detergent.

In one embodiment, the present invention is directed to a detergent composition comprising
a) an enzyme composition as described herein;
b) one or more surfactant, preferably, in a concentration of 0.2-65%, preferably 0.2-40%,
c) one or more builder, preferably, in a concentration of 0.01-25%, and
d) optionally one or more additional compound selected from the group consisting of additional enzyme different from the enzymes of the enzyme composition described under a), defoamer, polymer, bleaching system (bleach), rheology modifier, hydrotrope, softening agent, desiccant, whitening agent, buffer, preservative, anti-corrosion additive, dyestuff and fragrance; preferably wherein detergent composition, is a liquid, powder, pouch, or capsule detergent composition.

Preferably the detergent composition, preferably powder detergent composition, of the present invention comprises in addition to the enzyme composition as described herein one or more of the compounds selected from the group consisting of alcohol ethoxylate 7EO, Coco fatty acid C12-18, C12-C14-fatty alcohol ether sulfate (1-3 EO, preferably 2 EO), Linear alkyl benzene sulphonic acid, AcetateNa, CitrateNa, Na Silicate, Na Carbonate, Na Phospahte, Na Hydrogencarbonate, Zeolite4A, HEDP, MGDA, Na Sulfate, Na Chloride, optical brightener, and polymers and optionally Bleach activator and Percarbonate.

Preferably the detergent composition, preferably powder detergent composition, of the present invention comprises in addition to the enzyme composition as described herein
b) one or more surfactant selected from the group consisting of Alcohol ethoxylate 7EO, Coco fatty acid C12-18, C12-C14-fatty alcohol ether sulfate (1-3 EO, preferably 2 EO), Linear alkyl benzene sulphonic acid, preferably, in a concentration of 0.2-65%,
c) one or more builder selected from the group consisting of HEDP, MGDA, GLDA, and DTPMP, preferably, in a concentration of 0.01-25%, and
d) one or more compound selected from the group consisting of AcetateNa, CitrateNa, Na Silicate, Na Carbonate, Na Phospahte, Na Hydrogencarbonate, Zeolite4A, Na Sulfate, Na Chloride, optical brightener, and polymers, and optionally Bleach activator and Percarbonate.

Preferably the detergent composition, preferably liquid detergent composition, of the present invention comprises in addition to the enzyme composition as described herein one or more of the compounds selected from the group consisting of alcohol ethoxylate 7EO, Coco fatty acid C12-18, C12-C14-fatty alcohol ether sulfate (1-3 EO, preferably 2 EO), Linear alkyl benzene sulphonic acid, sulphonic acid, 1,2 Propandiol, Triethanolamine, Monoethanolamine, NaOH, Glycerol, Ethanol, Na citrate, and Polymer.

Preferably the detergent composition, preferably liquid detergent composition, of the present invention comprises in addition to the enzyme composition as described herein
b) one or more surfactant selected from the group consisting of Alcohol ethoxylate 7EO, Coco fatty acid C12-18, C12-C14-fatty alcohol ether sulfate (1-3 EO, preferably 2 EO), Linear alkyl benzene sulphonic acid, preferably, in a concentration of 0.2-65%,
c) one or more builder selected from the group consisting of HEDP, MGDA, GLDA, and DTPMP, preferably, in a concentration of 0.01-25%, and
d) one or more compound selected from the group consisting of sulphonic acid, 1,2 Propandiol, Triethanolamine, Monoethanolamine, NaOH, Glycerol, Ethanol, Na citrate, and Polymer.

In one embodiment, the enzyme composition described herein is included in a detergent composition comprising one or more, preferably all, compounds selected from the group consisting of (all percentages are w/w):
A formulation comprising the enzyme composition described herein, from 0.5 to 4%;
Anionic detersive surfactant (such as alkyl benzene sulphonate, alkyl ethoxylated sulphate and mixtures), from 8% to 15%;
Non-ionic detersive surfactant (such as alkyl ethoxylated alcohol), from 0.5% to 4%;
Cationic detersive surfactant (such as quaternary ammonium compounds), from 0 to 4%;
Other detersive surfactant (such as zwitterionic detersive surfactants, amphoteric surfactants and mixtures thereof), from 0% to 4%;
Carboxylate polymer (such as co-polymers of maleic acid and acrylic acid), from 1% to 4%;
Polyethylene glycol polymer (such as a polyethylene glycol polymer comprising poly vinyl acetate side chains), from 0.5% to 4%;
Polyester soil release polymer (such as Repel-o-tex from and/or Texcare polymers), from 0.1 to 2%;
Cellulosic polymer (such as carboxymethyl cellulose, methyl cellulose and combinations thereof), from 0.5% to 2%;
Other polymer (such as amine polymers, dye transfer inhibitor polymers, hexamethylenediamine derivative polymers, and mixtures thereof), from 0% to 4%;
Zeolite builder and phosphate builder (such as zeolite 4A and/or sodium tripolyphosphate), from 0% to 4 wt %;
Other builder (such as sodium citrate and/or citric acid), from 0% to 3%;
Carbonate salt (such as sodium carbonate and/or sodium bicarbonate), from 15% to 30%;
Silicate salt (such as sodium silicate), from 0% to 10%;
Filler (such as sodium sulphate and/or bio-fillers), from 10% to 40%;
Source of available oxygen (such as sodium percarbonate), from 10% to 20%;
Bleach activator (such as tetraacetylethylene diamine (TAED) and/or nonanoyloxybenzenesulphonate (NOBS), from 2% to 8%;
Bleach catalyst (such as oxaziridinium-based bleach catalyst and/or transition metal bleach catalyst), from 0% to 0.1%;
Other bleach (such as reducing bleach and/or pre-formed peracid), from 0% to 10%;
Chelant (such as ethylenediamine-N′N′-disuccinic acid (EDDS) and/or hydroxyethane diphosphonic acid (HEDP), from 0.2% to 1%;
Photobleach (such as zinc and/or aluminium sulphonated phthalocyanine), from 0% to 0.1%;
Hueing agent (such as direct violet 99, acid red 52, acid blue 80, direct violet 9, solvent violet 13 and any combination thereof), from 0% to 1%;
Brightener (such as brightener 15 and/or brightener 49), from 0.1% to 0.4%;
Fabric softener (such as montmorillonite clay and/or polydimethylsiloxane (PDMS)), from 0% to 4%;
Flocculant (such as polyethylene oxide), from 0% to 1%;
Suds suppressor (such as silicone and/or fatty acid), from 0% to 0.1%;
Perfume (such as perfume microcapsule, spray-on perfume, starch encapsulated perfume accords, perfume loaded zeolite, and any combination thereof), from 0.1% to 1%; and
Aesthetics (such as colored soap rings and/or colored speckles/noodles), from 0% to 1%; and
Optionally additional enzyme different from the enzymes of the enzyme composition described herein.

In another embodiment, the enzyme composition described herein is included in a detergent composition comprising one or more, preferably all, compounds selected from the group consisting of (all percentages are w/w):
A formulation comprising the enzyme composition described herein, from 0.5 to 4%;
Carboxyl group-containing polymer (comprising from about 60% to about 70% by mass of an acrylic acid-based monomer (A); and from about 30% to about 40% by mass of a sulfonic acid group-containing monomer (B); and wherein the average molecular weight is from about 23,000 to about 50,000 preferably in the range of from about 25,000 to about 38,000 as described in WO2014032269), from about 0.5 wt % to ab out 1.5 wt %;
Anionic detersive surfactant (such as alkyl benzene sulphonate, alkyl ethoxylated sulphate and mixtures thereof), from about 8 wt % to about 15 wt %;
Non-ionic detersive surfactant (such as alkyl ethoxylated alcohol) from about 0.5 wt % to 4 wt %;
Cationic detersive surfactant (such as quaternary ammonium compounds), from about 0 wt % to about 4 wt %;
Other detersive surfactant (such as zwitterionic detersive surfactants, amphoteric surfactants and mixtures thereof), from about 0 wt % to 4 wt %;
Carboxylate polymer (such as co-polymers of maleic acid and acrylic acid) from about 1 wt % to about 4 wt %;
Polyethylene glycol polymer (such as a polyethylene glycol polymer comprising poly vinyl acetate side chains), from about 0 wt % to about 4 wt %;
Polyester soil release polymer (such as Repel-O-Tex® and/or Texcare® polymers), from about 0.1 wt % to about 2 wt %;
Cellulosic polymer (such as carboxymethyl cellulose, methyl cellulose and combinations thereof) from about 0.5 wt % to about 2 wt %;
Other polymer (such as amine polymers, dye transfer inhibitor polymers, hexamethylenediamine derivative polymers, and mixtures thereof), from about 0 wt % to about 4 wt %;
Zeolite builder and phosphate builder (such as zeolite 4A and/or sodium tripolyphosphate), from about 0 wt % to about 4 wt %;
Other builder (such as sodium citrate and/or citric acid), from about 0 wt % to about 3 wt %;
Carbonate salt (such as sodium carbonate and/or sodium bicarbonate), from about 15 t % to about 30 wt %;
Silicate salt (such as sodium silicate), from about 0 wt % to about 10 wt %;
Filler (such as sodium sulphate and/or bio-fillers), from about 10 wt % to about 40 wt %;
Source of available oxygen (such as sodium percarbonate), from about 10 wt % to about 20 wt %;
Bleach activator (such as tetraacetylethylene diamine (TAED) and/or nonanoyloxybenzenesulphonate (NOBS), from about 2 wt % to about 8 wt %;
Bleach catalyst (such as oxaziridinium-based bleach catalyst and/or transition metal bleach catalyst), from about 0 wt % to about 0. 1 wt %;
Other bleach (such as reducing bleach and/or pre-formed peracid), from about 0 wt % to about 10 wt %;
Chelant (such as ethylenediamine-N′N′-disuccinic acid (EDDS) and/or hydroxyethane diphosphonic acid (HEDP), from about 0.2 wt % to about 1 wt %;
Photobleach (such as zinc and/or aluminium sulphonated phthalocyanine), from about 0 wt % to about 0. 1 wt %;
Hueing agent (such as direct violet 99, acid red 52, acid blue 80, direct violet 9, solvent violet 13 and any combination thereof), from about 0 wt % to about 0.5 wt %;
Brightener (such as brightener 15 and/or brightener 49), from about 0.1 wt % to about 0.4 wt %;
Fabric softener (such as montmorillonite clay and/or polydimethylsiloxane (PDMS)), from 0 wt % to 15 wt %;
Flocculant (such as polyethylene oxide), from 0 wt % to 1 wt %;
Suds suppressor (such as silicone and/or fatty acid), from 0 wt % to 0.1 wt %;
Perfume (such as perfume microcapsule, spray-on perfume, starch encapsulated perfume accords, perfume loaded zeolite, and any combination thereof), from 0.1 wt % to 1 wt %; and
Aesthetics (such as colored soap rings and/or colored speckles/noodles), from 0 wt % to 1 wt %, Optionally additional enzyme different from the enzymes of the enzyme composition described herein.

Further preferred detergent compositions comprise the components listed below (all percentages are w/w):
Aqua, Alcohol Ethoxy Sulfate, Alcohol Ethoxylate, Amino Oxide, Citrid Acid, C12-18 topped palm kernel fatty acid, Ethanol, 1,2 Propanediol, Sodium Formate, Calcium Chloride, Sodium hydroxide, Silicone Emulsion, Trans-sulphated EHDQ, an enzyme composition as described herein;
Linear sodium alkyl benzene sulfonate 8.8%, Ethoxylated fatty alcohol C12-18 (7 EO) 4.7%, Sodium soap 3.2%, Antifoam DC2-4248S 3.9%, Sodium aluminium silicate zeolite 4A 28.3%, Sodium carbonate 11.6%, Sodium salt of a copolymer from acrylic and maleic acid (Sokalan CP5) 2.4%, Sodium silicate 3.0%, Carboxymethylcellulose 1.2%, Dequest 2066 2.8%, Optical whitener 0.2%, Sodium sulfate 6.5%, an enzyme composition as described herein;
12% LAS, 11% AEO Biosoft N25-7 (NI), 7% AEOS (SLES), 6% MPG (monopropylene glycol), 3% ethanol, 3% TEA, 2.75% cocoa soap, 2. 75% soya soap, 2% glycerol, 2% sodium hydroxide, 2% sodium citrate, 1% sodium formate, 0.2% DTM PA and 0.2% PCA, an enzyme composition as described herein;
5-15% Anionic surfactants; <5% Non-ionic surfactants, Phosphonates, Soap; Enzymes, Optical brighteners, Benzisothiazolinone, Methylisothiazolinone, Perfumes, Alpha-isomethyl ionone, Citronellol, Geraniol, Linalool, an enzyme composition as described herein;
Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, Hydrogenated Castor Oil, Sodium Diethylenetriamine Pentamethylene Phosphonate, PEG/PPG-10/2 Propylheptyl Ether, Butyophenyl Methylpropional, Polyvinylpyridine-N-Oxide, Sorbitol, Glycerin, Ethanolamine, Sodium Hydroxide, Alpha-dsomethyllonone, Calcium Chloride, Geraniol, Linalool, Citronelllol, Tripropylene Glycol, Benzisothiazolinone, Dimethicone, Sodium Acetate, Colorant, Glyceryl Stearate, Hydroxyethylcellulose, Silica, an enzyme composition as described herein;
Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Perfume, Polyvinylpyridine-N-Oxide, Sorbitol, Calcium Chloride, Glycerin, Sodium Acetate, Colorant, an enzyme composition as described herein;
Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Perfume, Sorbitol, Calcium Chloride, Glycerin, Sodium Acetate, Colorant, an enzyme composition as described herein;
Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Sorbitol, Calcium Chloride, Glycerin, Glycosidase, Sodium Acetate, Silica, an enzyme composition as described herein;
Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, C12-C14 Pareth-7, Hydrogenated Castor Oil, Sodium Diethylenetriamine Pentamethylene Phosphonate, PEG/PPG-10/2 Propylheptyl Ether, Butyophenyl Methylpropional, Fluorescent Brightener, Sorbitol, Glycerin, Ethanolamine, Sodium Hydroxide, Alpha-Isomethyl lonone, Calcium Chloride, Geraniol, Linalool, Citronelllol, Tripropylene Glycol, Sodium Chloride, Benzisothiazolinone, Dimethicone, Glycosidase, Sodium Acetate, Colorant, Glyceryl Stearate, Hydroxyethylcellulose, Silica, an enzyme composition as described herein;
15-30% Anionic surfactants, Non-ionic surfacts, 5-15% Soap, <5% Polycarboxylates, Perfume, Phosphates, Optical Brighteners, an enzyme composition as described herein;
15-30% Anionic Surfactants, 5-15% Non-Ionic Surfactants, Soap, Benzisothiazolinone, Methylisothiazolinone, Perfumes, an enzyme composition as described herein;
11% LAS, 2% AS/AEOS, 2% soap, 3% AEO, 15.15% sodium carbonate, 3% sodium silicate, 18.75% zeolite, 0.15% chelant, 2% sodium citrate, 1.65% AA/MA copolymer, 2.5% CMC and 0.5% SRP, an enzyme composition as described herein;
16.5% LAS, 15% zeolite, 12% sodium disilicate, 20% sodium carbonate, 1% sokalan, 35.5% sodium sulfate, an enzyme composition as described herein;
15-30% Anionic surfactants, <5% Nonionic surfactants, Phosphonates, Polycarboxylates, Zeolites;
Enzymes, Perfumes, Hexyl cinnamal, an enzyme composition as described herein;
15-30% of the following: anionic surfactants, oxygen-based bleaching agent and zeolites, less than 5% of the following: non-ionic surfactants, phosphonates, polycarboxylates, soap, Further ingredients: Perfumes, Hexyl cinnamal, Benzyl salicylate, Linalool, optical brighteners, Enzymes and Citronellol, an enzyme composition as described herein;
Water, Alcohol Ethoxysulfate, Diethylene Glycol, Alcohol Ethoxylate, Ethanolamine, Linear Alkyl Benzene Sulfonate, Sodium Fatty Acids, Polyethyleneimine Ethoxylate, Citric Acid, Sodium Cumene Sulfonate, Propylene Glycol, DTPA, Disodium Diaminostilbene Disulfonate, Dipropylethyl Tetramine, Sodium Hydroxide, Sodium Formate, Calcium Formate, Dimethicone, Liquitint™, Hydrogenated Castor Oil, Fragrance, an enzyme composition as described herein;
Linear alkylbenzene sulfonate, propylene glycol, citric acid, sodium hydroxide, ethanolamine, ethanol, alcohol sulfate, polyethyleneimine ethoxylate, sodium fatty acids, diquaternium ethoxysulfate, diethylene glycol, laureth-9, alkyldimethylamine oxide, fragrance, disodium diaminostilbene disulfonate, DTPA, sodium formate, calcium formate, polyethylene glycol 4000, Liquitint™ Blue, dimethicone, an enzyme composition as described herein;
Water, sodium alcoholethoxy sulfate, propylene glycol, ethanol, linear alkylbenzene sulfonate sodium, salt, polyethyleneimine ethoxylate, diethylene glycol, trans sulfated & ethoxylated hexamethylene diamine, alcohol ethoxylate, linear alkylbenzene sulfonate, MEA salt, sodium formate, sodium alkyl sulfate, DTPA, amine oxide, calcium formate, disodium diaminostilbene, disulfonate, dimethicone, benzisothiazolinone, an enzyme composition as described herein;
Water, alcoholethoxy sulfate, linear alkylbenzene sulfonate, diethylene glycol, propylene glycol, ethanolamine, citric acid, alcohol sulfate, sodium hydroxide, polyethyleneimine, ethoxylate, sodium fatty acids, ethanol, Laureth-9, diquaternium ethoxysulfate, lauramine oxide, sodium cumene, sulfonate, fragrance, DTPA, disodium, diaminostilbene, disulfonate, sodium formate, disodium distyrylbiphenyl, disulfonate, calcium formate, polyethylene glycol 4000, pectinase, Liquitint™ Blue, dimethicone, an enzyme composition as described herein;
Water, alcoholethoxy sulfate, propylene glycol, sodium fatty acids, laurtrimonium chloride, ethanol, sodium hydroxide, sodium cumene sulfonate, citric acid, ethanolamine, diethylene glycol, silicone polyether, fragrance, polyethylene-imine ethoxylate, Laureth-9, DTPA, polyacrylamide quaternium chloride, disodium diaminostilbene disulfonate, sodium formate, Liquitint™ Orange, dipropylethyl tetraamine, dimethicone, an enzyme composition as described herein;
Water, sodium alcoholethoxy sulfate, sodium alkyl sulfate, MEA citrate, linear alkylbenzene sul-fonate, MEA salt, propylene glycol, diethylene glycol, polyethyleneimine ethoxylate, ethanol, sodium fatty acids, ethanolamine, lauramine oxide, Laureth-9, DTPA, sodium cumene sulfonate, sodium formate, calcium formate, linear alkylbenzene sulfonate, sodium salt, alcohol sulfate, sodium hydroxide, diquaternium ethoxysulfate, fragrance, pectinase, disodium diaminostilbene disulfonate, benzisothiazolinone, Liquitint™ Blue, dimethicone, dipropylethyl tetraamine, an enzyme composition as described herein;
Water, Sodium alcoholethoxy sulfate, MEA citrate, Sodium Alkyl Sulfate, alcohol ethoxylate, linear alkylbenzene sulfonate, MEA salt, sodium fatty acids, polyethyleneimine ethoxylate, diethylene glycol, propylene glycol, diquaternium ethoxysulfate, polyethyleneimine, ethoxylate propoxylate, ethanol, sodium cumene sulfonate, fragrance, DTPA, disodium diaminostilbene disulfonate, sodium formate, calcium formate, Lauramine oxide, Liquitint™ Blue, Dimethicone/polydimethyl silicone, an enzyme composition as described herein;
Water, alcoholethoxy sulfate, linear alkylbenzene sulfonate, alcohol ethoxylate, citric acid, Ethanolamine, sodium fatty acids, diethylene glycol, propylene glycol, sodium hydroxide, polyethyleneimine ethoxylate, silicone polyether, ethanol, sodium cumene sulfonate, diquaternium ethoxysulfate, Laureth-9, fragrance, DTPA, disodium diaminostilbene disulfonate, disodium distyrylbiphenyl disulfonate, sodium formate, calcium formate, Liquitint™ Orange, dimethicone, polyacrylamide quaternium chloride, dipropylethyl tetraamine, an enzyme composition as described herein;
Water, alcoholethoxy sulfate, diethylene glycol, monoethanolamine citrate, sodium formate, propylene glycol, linear alkylbenzene sulfonates, ethanolamine, ethanol, polyethyleneimine ethoxylate, benzisothiazolin, calcium formate, citric acid, diethylenetriamine pentaacetate sodium, dimethicone, diquaternium ethoxysulfate, disodium dia-minostilbene disulfonate, Laureth-9, sodium cumene sulfonate, sodium fatty acids, an enzyme composition as described herein;
Water, alcoholethoxy sulfate, MEA Citrate, alcohol sulfate, Alcohol ethoxylate, Linear alkylbenzene sulfonate MEA, sodium fatty acids, polyethyleneimine ethoxylate, diethylene glycol, propylene glycol, diquaternium ethoxysulfate, polyethyleneimine ethoxylate propoxylate, ethanol, sodium cumene sulfonate, fragrance, DTPA, disodium diaminostilbene disulfonate, sodium formate, calcium formate, lauramine oxide, Liquitint™ Blue, dimethicone, an enzyme composition as described herein;
Water, sodium alcoholethoxy sulfate, MEA Citrate, Linear alkylbenzene sulfonate: sodium salt, Alcohol ethoxylate, Linear alkylbenzene sulfonate: MEA salt, sodium fatty acids, polyethyleneimine ethoxylate, diethylene glycol, propylene glycol, diquaternium ethoxysulfate, polyethyleneimine ethoxylate propoxylate, ethanol, sodium cumene sulfonate, citric acid, DTPA, disodium diaminostilbene disulfonate, sodium formate, calcium formate, dimethicone, an enzyme composition as described herein;
Water, alcohol ethoxylate sulfate, linear alkylbenzene sulfonate Sodium/Mea salts, propylene glycol, diethylene glycol, sodium formate, ethanol, sodium fatty acids, fragrance, lauramine oxide, DTPA, Polyethylene amine ethoxylate, calcium formate, disodium diaminostilbene disulfonate, dimethicone, tetramine, Liquitint™ Blue, an enzyme composition as described herein;
Linear alkylbenzene sulfonates, C12-16 Pareth-9, propylene glycol, alcoholethoxy sulfate, water, polyethyleneimine ethoxylate, glycerine, fatty acid salts, PEG-136 polyvinyl acetate, ethylene Diamine disuccinic salt, monoethanolamine citrate, sodium bisulfite, diethylenetriamine pentaacetate sodium, disodium distyrylbiphenyl disulfonate, calcium formate, exyloglucanase, sodium formate, hydrogenated castor oil, dyes, benzisothiazolin, perfume, an enzyme composition as described herein;
Deionized water, Dipropylene Glycol Butyl Ether, Sodium Alkyl Sulfate, Hydrogen Peroxide, Ethanol, Magnesium Sulfate, Alkyl Dimethyl Amine Oxide, Citric Acid, Sodium Hydroxide, Trimethoxy Benzoic Acid, Fragrance, an enzyme composition as described herein;
Water, Alkyl Ethoxylate, Linear Alkylbenzenesulfonate, Hydrogen Peroxide, Diquaternium Ethoxysulfate, Ethanolamine, Disodium Distyrylbiphenyl Disulfonate, tetrabutyl Ethylidinebisphenol, F&DC Yellow 3, Fragrance, an enzyme composition as described herein;
Sodium percarbonate, sodium sulfate, sodium carbonate, sodium aluminosilicate, nonanoyloxy benzene sulfonate, sodium polyacrylate, water, sodium alkylbenzenesulfonate, DTPA, polyethylene glycol, sodium palmitate, modified starch, FD&C Blue 1, fragrance, an enzyme composition as described herein;
Water, Alkyl Ethoxylate, MEA Borate, Linear Alkylbenzenesulfonate, Propylene Glycol, Diquaternium Ethoxysulfate, Calcium Chlorideenzyme, Ethanolamine, Benzoisothiazolinone, Sodium Citrate, Sodium Hydroxide, Fragrance, an enzyme composition as described herein;
Water, Alkyl Amine Oxide, Dipropylene Glycol Phenyl Ether, Hydrogen Peroxide, Citric Acid, Ethylene Diamine Disuccinic Acid Sodium salt, Sodium Alkyl Sulfate, Fragrance, an enzyme composition as described herein;
Sodium bicarbonate, sodium carbonate, sodium percarbonate, alcohol ethoxylate, sodium chloride, maleic/acrylic copolymer, nonanoyloxy benzene sulfonate, sodium sulfate, colorant, diethylenetriamine pentaacetate sodium salt, hydrated aluminosilicate (zeolite), polyethylene glycol, sodium alkylbenzene sulfonate, sodium palmitate, starch, water, fragrance, an enzyme composition as described herein;
Polyvinyl Alcoholpouch film, wherein there is packed a liquid part and a powder part: Liquid Ingredients: Dipropylene Glycol, diquaternium Ethoxysulfate, Water, Glycerin, Liquitint™ Orange, an enzyme composition as described herein;
power ingredients: sodium percarbonate, nonanoyloxy benzene sulfonate, sodium carbonate, sodium sulfate, sodium aluminosilicate, sodium polyacrylate, sodium alkylbenzenesulfonate, maleic/acrylic copolymer, water, polyethylene glycol, sodium palmitate, modified starch, glycerine, DTPA, fragrance, an enzyme composition as described herein;
Water, sodium alcoholethoxy sulfate, linear alkyl benzene sulfonate, sodium/MEA salts, MEA citrate, propylene glycol, polyethyleneimine ethoxylate, ethanol, diethylene glycol, polyethyleneimine propoxyethoxylate, sodium fatty acids, sodium cumene sulfonate, DTPA, fragrance, disodium diaminostilbene disulfonate, calcium formate, sodium formate, dimethicone, Liquitint™ Blue, an enzyme composition as described herein;
Sodium Carbonate, Sodium Aluminosilicate, Sodium Sulfate, Linear Alkylbenzene Sulfonate, Bentonite, Water, Sodium Percarbonate, Sodium Polyacrylate, Silicate, Alkyl Sulfate, Nonanoyloxybenzenesulfonate, DTPA, Polyethylene Glycol 4000, Silicone, Ethoxylate, fragrance, Polyethylene Oxide, Palmitic Acid, Disodium Diaminostilbene Disulfonate, Liquitint™ Red, FD&C Blue 1, an enzyme composition as described herein;
Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimine, propoxyethoxylate, diquaternium ethoxysulfate, alcohol sulfate, dimethicone, fragrance, sodium fatty acids, DTPA, sodium bisulfite, disodium diaminostilbene disulfonate, castor oil, calcium formate, MEA, styrene acrylate copolymer, sodium formate, Liquitint™ Blue, an enzyme composition as described herein;
Water, sodium alcoholethoxy sulfate, MEA citrate, linear alkyl benzene sulfonate: sodium/MEA salts, propylene glycol, ethanol, diethylene glycol, polyethyleneimine propoxyethoxylate, polyethyleneimine ethoxylate, alcohol sulfate, dimethicone, fragrance, sodium fatty acids, DTPA, sodium bisulfite, disodium diaminostilbene disulfonate, castor oil, calcium formate, MEA, styrene acrylate copolymer, propanaminium propanamide, sodium formate, Liquitint™ Blue, an enzyme composition as described herein;
Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimin propoxyethoxylate, diquaternium ethoxysulfate, alcohol sulfate, dimethicone, fragrance, sodium fatty acids, DTPA, sodium bisulfite, disodium diaminostilbene disulfonate, castor oil, calcium formate, MEA, styrene acrylate copolymer, propanaminium propanamide, sodium formate, Liquitint™ Blue, an enzyme composition as described herein;
Sodium Carbonate, Sodium Aluminosilicate, Alkyl Sulfate, Sodium Sulfate, Linear Alkylbenzene Sulfonate, Water, Sodium polyacrylate, Silicate, Ethoxylate, Sodium percarbonate, Polyethylene Glycol 4000, Disodium Diaminostilbene Disulfonate, Silicone, an enzyme composition as described herein;
Sodium Carbonate, Sodium Aluminosilicate, Sodium Sulfate, Linear Alkylbenzene Sulfonate, Alkyl Sulfate, Sodium Percarbonate, Water, Sodium Polyacrylate, Silicate, Nonanoyloxybenzenesulfonate, Ethoxylate, Polyethylene Glycol 4000, Fragrance, DTPA, Disodium Diaminostilbene Disulfonate, Palmitic Acid, Silicone, an enzyme composition as described herein;
Sodium Carbonate, Sodium Aluminosilicate, Sodium Sulfate, Linear Alkylbenzene Sulfonate, Water, Nonanoyloxybenzenesulfonate, Alkyl Sulfate, Sodium Polyacrylate, Silicate, Sodium Percarbonate, Ethoxylate, Polyethylene Glycol 4000, Fragrance, DTPA, Palmitic Acid, Disodium Diaminostilbene Disulfonate, Silicone, an enzyme composition as described herein;
Sodium Carbonate, Sodium Aluminosilicate, Sodium Sulfate, Sodium Percarbonate, Alkyl Sulfate, Linear Alkylbenzene Sulfonate, Water, Nonanoyloxybenzenesulfonate, Sodium Polyacrylate, Silicate, Ethoxylate, Polyethylene Glycol 4000, DTPA, Fragrance, Palmitic Acid, Disodium, Diaminostilbene Disulfonate, FD&C Blue 1, Silicone, Alkyl Ether Sulfate, an enzyme composition as described herein;
Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate, Ethoxylate, Polyethylene Glycol 4000, Fragrance, DTPA, Palmitic Acid, Disodium Diaminostilbene Disulfonate, Silicone, FD&C Blue 1, Alkyl Ether Sulfate, an enzyme composition as described herein;
Sodium Carbonate, Sodium Aluminosilicate, Sodium Sulfate, Linear Alkylbenzene Sulfonate, Sodium Percarbonate, Alkyl Sulfate, Water, Sodium Polyacrylate, Silicate, Nonanoyloxybenzenesulfonate, Ethoxylate, Polyethylene Glycol 4000, DTPA, Fragrance, Disodium Diaminostilbene Disulfonate, Silicone, FD&C Blue 1, an enzyme composition as described herein;
Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, Ethanol, sodium cumene sulfonate, fragrance, DTPA, Sodium bisulfate, disodium diaminostilbene disulfonate, sodium formate, calcium formate, Lauramine oxide, Liquitint™ Blue, Dimethicone/polydimethyl silicone, an enzyme composition as described herein;
Water, sodium alcoholethoxy sulfate, linear alkyl benzene sulfonate: sodium/MEA salts, MEA citrate, propylene glycol, polyethyleneimine ethoxylate, fragrance, ethanol, diethylene glycol, polyethyleneimine propoxyethoxylate, alcohol sulfate, sodium fatty acids, DTPA, disodium diaminostilbene disulfonate, MEA, sodium formate, dimethicone, Liquitint™ Blue, tetramine, an enzyme composition as described herein;
Water, Sodium alco-holethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, ethanol, sodium cumene sulfonate, fragrance, DTPA, Sodium bisulfate, disodium diaminostilbene disulfonate, sodium formate, calcium formate, Lauramine oxide, Liquitint™ Blue, Dimethicone/polydimethyl silicone, an enzyme composition as described herein;
Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate Ethoxylate, Polyethylene Glycol 4000, Fragrance, DTPA, Palmitic Acid, Disodium Diaminostilbene Disulfonate, Silicone, FD&C Blue 1, Alkyl Ether Sulfate, an enzyme composition as described herein; or
Aqua, dodecylbenzenesulfonsaure, laureth-11, peg-75 lanolin, propylene glycol, alcohol denat., potassium soyate, potassium hydroxide, disodium cocoamphodiacetate, ethylendiamine triacetate cocosalkyl acetamide, parfum, zinc ricinoleate, sodium chloride, benzisothiazolinone methylisothiazolinone, ci 16255, benzyl alcohol, an enzyme composition as described herein;
Preferably, the compositions including a protease further comprise 4-FPBA and/or a peptide aldehyde protease inhibitor, most preferably Z-GAY or Z-VAL.

The enzyme composition described herein can be comprised in one of the following detergent compositions.

| | | wt % in |
|---|---|---|
| ES1-C | | formulation |
| Maranil DBS/LC | linear alkylbenzene sulfonate, | 5.5 |
| | anionic surfactant | |
| Edenor coco fatty acid | C₁₂-C₁₈ coco fatty acid | 2.4 |
| Lutensol AO7 | alkyl polyethyleneglycol ether, | 5.5 |
| | non-ionic surfactant | |
| Texapon N70 | sodium laureth sulfate 2EO, | 5.5 |
| | anionic surfactant | |
| 1,2 propylene glycol | | 6.0 |
| C₂H₅OH | | 2.0 |
| NaOH | | 2.2 |
| Citrate | | 3.0 |
| a formulation comprising | | 0.5 to 4 |
| the enzyme composition | | |
| described herein | | |
| pH | | 8-8.5 |
| Water | | up to 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| Na-Citrat | 1.5 |
| NaOH 50% | |
| Propylenglykol | 10.0 |
| a formulation comprising the | 0.5 to 4 |
| enzyme composition described herein | |
| water | Ad 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| MGDA | 1.5 |
| NaOH 50% | |
| Propylenglykol | 10.0 |
| a formulation comprising the enzyme | 0.5 to 4 |
| composition described herein | |
| Water | Ad 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| GLDA | 1.5 |
| NaOH 50% | |
| Propylenglykol | 10.0 |
| a formulation comprising the enzyme | 0.5 to 4 |
| composition described herein | |
| Water | Ad 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| Phosphonate (e.g., HEDP or DTPMP) | 1.5 |
| NaOH 50% | |
| Propylenglykol | 10.0 |
| a formulation comprising the enzyme | 0.5 to 4 |
| composition described herein | |
| Water | Ad 100% |

% wt in formulation

| | | | | | | | | | | | | |
|---|---|---|---|---|---|---|---|---|---|---|---|---|
| Alcohol ethoxylate 7EO | 0.6 | 0 | 1 | 0 | 0 | 5.2 | 1.2 | 5 | 4 | 0.5 | 0.5 | 0 |
| Coco fatty acid C12-18 | 1.2 | 0 | 0 | 0 | 0 | 0 | 0 | 0 | 0 | 0.3 | 0 | 0.6 |
| C12-C14-fatty alcohol | 1.5 | 0 | 0 | 0 | 0 | 6 | 0 | 3.9 | 4.4 | 1.6 | 0 | 0 |
| ether sulfate (2 EO) | | | | | | | | | | | | |
| Linear alkyl benzene | 12.1 | 11.2 | 13.6 | 21.9 | 18.7 | 12.7 | 7.6 | 12.1 | 11.5 | 12.2 | 6.5 | 10.4 |
| sulphonic acid | | | | | | | | | | | | |
| Bleach activator | 0 | 0 | 0 | 0 | 0 | 0 | 0.2 | 9.5 | 9.5 | 0.5 | 0.8 | 2.2 |
| Percarbonate | 0 | 0 | 0 | 0 | 0 | 0 | 3.6 | 19.4 | 16.6 | 2.2 | 11.5 | 5.8 |
| AcetateNa | 0 | 0 | 0 | 0.1 | 0 | 0.1 | 0 | 6.7 | 7.1 | 0.3 | 1 | 0.7 |
| CitrateNa | 0 | 0 | 0 | 0 | 0 | 14 | 0 | 1.6 | 8.2 | 0.3 | 0.9 | 1.7 |
| Na Silicate | 27.9 | 5.8 | 6.6 | 2 | 15 | 20.3 | 3.6 | 11.3 | 16.4 | 10.2 | 9.1 | 16.5 |
| Na Carbonate | 17.2 | 35 | 37.3 | 30.1 | 37 | 1 | 21.6 | 8.7 | 1.4 | 8 | 22.9 | 14.8 |
| Na Phospahte | 0 | 0 | 0 | 14 | 0.3 | 0 | 0 | 0 | 0 | 0 | 0 | 0 |
| Na Hydrogencarbonate | 0.7 | 0.9 | 0.5 | 2.7 | 0.4 | 10.5 | 0.2 | 2.8 | 1.6 | 0.8 | 0.5 | 0.5 |
| Zeolite4A | 4.2 | 0.1 | 5.1 | 10.2 | 1.8 | 11.6 | 1.6 | 1.4 | 2.4 | 1.6 | 1.8 | 2.3 |
| HEDP | 0 | 0 | 0 | 0 | 0 | 0.13 | 0 | 0.27 | 0.16 | 0 | 0 | 0.17 |
| MGDA | 0 | 1.1 | 0 | 0 | 0 | 0 | 0 | 0 | 0 | 0 | 0 | 0 |
| Cellulase * | 0-0.5 | 0-0.5 | 0-0.5 | 0-0.5 | 0-0.5 | 0-0.5 | 0-0.5 | 0-0.5 | 0-0.5 | 0-0.5 | 0-0.5 | 0-0.5 |
| Lipase | 0-0.4 | 0-0.4 | 0-0.4 | 0-0.4 | 0-0.4 | 0-0.4 | 0-0.4 | 0-0.4 | 0-0.4 | 0-0.4 | 0-0.4 | 0-0.4 |
| Mannanase * | 0-0.4 | 0-0.4 | 0-0.4 | 0-0.4 | 0-0.4 | 0-0.4 | 0-0.4 | 0-0.4 | 0-0.4 | 0-0.4 | 0-0.4 | 0-0.4 |
| Protease */** | 0-1.5 | 0-1.5 | 0-1.5 | 0-1.5 | 0-1.5 | 0-1.5 | 0-1.5 | 0-1.5 | 0-1.5 | 0-1.5 | 0-1.5 | 0-1.5 |
| Amylase * | 0-0.5 | 0-0.5 | 0-0.5 | 0-0.5 | 0-0.5 | 0-0.5 | 0-0.5 | 0-0.5 | 0-0.5 | 0-0.5 | 0-0.5 | 0-0.5 |
| Na Sulfate | 30.8 | 1.3 | 33 | 11 | 22 | 3 | 51 | 4 | 6 | 57 | 38 | 37 |
| Na Chloride | 0.2 | 43 | 0.1 | 0 | 0.1 | 0.1 | 1 | 1 | 0.5 | 1.2 | 0.2 | 1 |
| optical brightener | 0.02 | 0 | | 0.1 | 0.06 | | | 0.29 | 0.1 | 0.23 | 0.13 | 0.19 |
| Polymers | 1 | 0 | 0.2 | 2 | 0.5 | 3 | 2.2 | 9.2 | 2.2 | 0.7 | | 0.4 |

** preferably, in case a protease is present, the composition preferably comprises a protease inhibitor, preferably selected from phenylboronic acid (preferably 4-FPBA) or a peptide aldehyde or a bisulfite adduct or acetal thereof (preferably a tripeptide aldehyde, preferably, Z-GAY or Z-VAL.
* preferably, amylase, protease, mannanase, and cellulase as described herein for the enzyme composition.

% wt in
formulation

| | | | | | | | | | | | | | | |
|---|---|---|---|---|---|---|---|---|---|---|---|---|---|---|
| alcohol | 5.40 | 10.80 | 12.40 | 7.30 | 1.60 | 7.60 | 3.80 | 0.30 | 13.30 | 8.00 | 5.70 | 20.00 | 9.20 | 29.00 |
| ethoxylat 7EO | | | | | | | | | | | | | | |
| Coco fatty | 2.40 | 3.10 | 3.20 | 3.20 | 3.50 | 6.40 | 2.80 | 3.00 | 1.70 | 1.80 | 2.50 | 5.00 | 8.60 | 10.40 |
| acid C12-18 | | | | | | | | | | | | | | |
| C12-C14-fatty | 5.40 | 8.80 | 7.10 | 7.10 | 5.40 | 14.00 | 2.80 | 4.50 | 3.90 | 4.10 | | 10.00 | 22.20 | |
| alcohol ether | | | | | | | | | | | | | | |
| sulfate (2 EO) | | | | | | | | | | | | | | |
| Linear alkyl | 5.50 | 0.00 | 14.50 | 15.50 | 10.70 | 0.00 | 6.30 | 5.43 | 11.45 | 5.90 | 10.10 | 10.00 | 28.00 | 27.00 |
| benzene | | | | | | | | | | | | | | |
| sulphonic acid | | | | | | | | | | | | | | |
| 1,2 Propandiol | 6.00 | 3.50 | 8.70 | 8.70 | 1.10 | 7.80 | 0.50 | | 2.50 | 0.40 | 6.00 | 10.00 | 7.00 | 7.00 |
| Triethanol- | | | | | | | | | | 8.10 | | | | |
| amine | | | | | | | | | | | | | | |
| Monoethanol- | | | 4.00 | 4.30 | 0.30 | | 0.40 | 1.80 | | | | | 8.00 | 7.00 |
| amine | | | | | | | | | | | | | | |
| NaOH | 2.20 | 1.10 | | | | 1.00 | | | 2.20 | | 3.30 | 1.50 | | |
| Glycerol | | 0.80 | 3.00 | 2.80 | | | | 0.60 | 0.20 | 1.90 | | | 7.00 | 10.00 |
| Ethanol | 2.00 | | | | 0.38 | 0.39 | | | 1.84 | | | | | |
| Na citrate | 3.00 | 2.80 | 3.40 | 2.10 | 7.40 | 5.40 | 4.60 | 3.30 | 3.30 | 1.40 | | 1.50 | | |
| Polymer | 0-5% | 0-5% | 0-5% | 0-5% | 0-5% | 0-5% | 0-5% | 0-5% | 0-5% | 0-5% | 0-5% | 0-5% | 0-5% | 0-5% |
| Protease */** | 0-1% | 0-1% | 0-1% | 0-1% | 0-1% | 0-1% | 0-1% | 0-1% | 0-1% | 0-1% | 0-1% | 0-3% | 0-3% | 0-3% |
| Amylase * | 0-0.5% | 0-0.5% | 0-0.5% | 0-0.5% | 0-0.5% | 0-0.5% | 0-0.5% | 0-0.5% | 0-0.5% | 0-0.5% | 0-0.5% | 0-0.5% | 0-0.5% | 0-0.5% |
| Cellulase * | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% |
| Lipase | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% |
| Mannanase * | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% |
| Pectat | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% |
| Lyase | | | | | | | | | | | | | | |
| water | to 100 | to 100 | to 100 | to 100 | to 100 | to 100 | to 100 | to 100 | to 100 | to 100 | to 100 | to 100 | to 100 | to 100 |

** preferably, in case a protease is present, the composition preferably comprises a protease inhibitor, preferably selected from phenylboronic acid (preferably 4-FPBA) or a peptide aldehyde or a bisulfite adduct or acetal thereof (preferably a tripeptide aldehyde, preferably, Z-GAY or Z-VAL.
* preferably, amylase, protease, mannanase, and cellulase as described herein for the enzyme composition.

### METHODS OF USE

The present invention is also directed to the use of an enzyme composition as described herein in a cleaning process such as laundry or hard surface cleaning, preferably for home care or I&I cleaning including manual and automated dish washing as well as medical device cleaning. Thus, the present invention also refers to the use of an enzyme composition as described herein for providing a detergent composition with improved enzyme stability and/or for providing a detergent composition with improved wash performance, preferably on complex stains.

Thus, the present invention therefore also refers to a method for cleaning, preferably laundry or hard surface cleaning, comprising the step of contacting on object, preferably a textile or a hard surface, with a composition comprising an enzyme composition as described herein, preferably wherein the composition comprises at least one additional detergent component, preferably a surfactant and/or a builder.

Further, the present invention also refers to a method for improving enzyme stability in a detergent composition and/or for improving wash performance of a detergent composition, preferably on complex stains, preferably on complex stains on a textile or fabric, comprising the step of formulating an enzyme composition as described herein in a detergent composition, preferably compared to an enzyme composition different to the one as described herein, preferably at low temperature, preferably at 30 °C or at 40 °C, preferably at 30 °C, preferably determined in ES1-C detergent as described herein.

Further, the present invention also refers to a method of removal of complex stains using the composition as described herein, preferably from a textile or fabric, preferably at low temperature, preferably at 30 °C or at 40 °C, preferably at 30 °C.

Further, the present invention also refers to a method of using the composition as described herein for providing a synergistic cleaning effect, preferably on complex stains, preferably at low temperature, preferably at 30 °C or at 40 °C, preferably at 30 °C, preferably at low temperature, preferably at 30 °C or at 40 °C, preferably at 30 °C, preferably as determined in ES1-C detergent as described herein, preferably compared to an enzyme composition different to the one as described herein.

Further, the present invention also refers to a method of using the composition as described herein for providing a synergistic cleaning effect on complex stains on a textile or fabric, preferably at low temperature, preferably at 30 °C or at 40 °C, preferably at 30 °C, preferably at low temperature, preferably at 30 °C or at 40 °C, preferably at 30 °C, preferably as determined in ES1-C detergent as described herein, preferably compared to an enzyme composition different to the one as described herein.

### PREFERRED EMBODIMENTS

Particularly, preferred herein is:
1. A composition comprising an amylase and one or more, preferably two or more enzymes selected from the group consisting of protease, mannanase, and cellulase, wherein
   a. the amylase is an amylase variant of a parent amylase, wherein said amylase variant comprises
      (i) an amino acid substitution at position 25 according to the numbering of SEQ ID NO: 2,
      (ii) an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2,
      (iii) at least 80%, preferably at least 91% identity, but less than 100% sequence identity with any of SEQ ID NO: 1-15, preferably SEQ ID NO: 1,
   b. the protease is selected from
      I. a protease variant of a parent protease, wherein the protease variant comprises
         (i) an amino acid sequence comprising compared to SEQ ID NO: 16 at least two additional negative charges in the loop region of residues 98 to 104 according to the numbering of SEQ ID NO: 17, and
         (ii) an amino acid sequence which is at least 80%, but less than 100% identical to any of SEQ ID NO: 16-34, preferably SEQ ID NO: 16, or
      II. a protease variant of a parent protease, wherein the protease variant comprises
         (i) an amino acid substitution at one or more amino acid positions corresponding to amino acid positions selected from the group consisting of 3, 4, 9, 19, 22, 43, 62, 63, 76, 87, 99, 101, 103, 104, 116, 118, 128, 129, 130, 156, 160, 167, 170, 182, 185, 188, 194, 205, 213, 217, 218, 232, 245, 256, 259, 262, and 271 according to the numbering of SEQ ID NO: 17 and an amino acid sequence which is at least 60%, but less than 100% sequence identity with SEQ ID NO: 22, or
         (ii) an amino acid substitution at one or more amino acid positions corresponding to amino acid positions selected from the group consisting of 24, 53, 76, 78, 101, 128, and 217 according to the numbering of SEQ ID NO: 17 and an amino acid sequence which is at least 80%, but less than 100% sequence identity with SEQ ID NO: 17, or
         (iii) an amino acid substitution at one or more amino acid positions corresponding to amino acid positions selected from the group consisting of 9, 89, 130, 133, 144, 159, 183, 189. 217, 224, 238, 243, 252, 259, 270 and 271 according to the numbering of SEQ ID NO: 19 and an amino acid sequence which is at least 80%, but less than 100% sequence identity with SEQ ID NO: 19, or
         (iv) an amino acid sequence which has at least 80% sequence identity with SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31,
   c. the mannanase is selected from
      I. a mannanase variant of a parent mannanase, wherein the mannanase variant comprises
         (i) one or more amino acid substitution selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and
         (ii) an amino acid sequence which is at least 80%, but less than 100% identical to a sequence according to positions 31-490 of SEQ ID NO: 35, or
      II. a mannanase variant of a parent mannanase, wherein the mannanase variant comprises
         (i) an amino acid substitution at one or more amino acid positions corresponding to amino acid positions selected from the group consisting of 24, 123, and 256 according to the numbering of SEQ ID NO: 39 and an amino acid sequence which is at least 80%, but less than 100% identical to SEQ ID NO: 39, or
         (ii) an amino acid substitution at one or more amino acid positions corresponding to amino acid positions selected from the group consisting of 10, 19, 30, 38, 59, 60, 67, 97, 103, 129, 143, 167, 168, 184, 225, 228, 235, 244, 258, 261, and 298 according to the numbering of SEQ ID NO: 38 and an amino acid sequence which is at least 80%, but less than 100% identical to SEQ ID NO: 38, or
         (iii) an amino acid sequence which is at least 60% identical to SEQ ID NO: 40, or
         (iv) an amino acid substitution at one or more amino acid positions corresponding to amino acid positions selected from the group consisting of 30, 81, 93, 95, 118, 138, 139, 200, 202, 210, 260, 283, 288, 294, and 295 according to the numbering of SEQ ID NO: 40 and an amino acid sequence which is at least 80%, but less than 100% identical to SEQ ID NO: 40,
   d. the cellulase is selected from
      I. a cellulase variant comprising an amino acid sequence which is at least 80% identical to SEQ ID NO: 42, or
      II. a cellulase variant comprising an amino acid sequence which is at least 80% identical to SEQ ID NO: 46, SEQ ID NO: 43, SEQ ID NO: 45, SEQ ID NO: 44, or SEQ ID NO: 47.
2. The composition according to embodiment 1, wherein the composition comprises an amylase and one or more, preferably two or more enzymes selected from the group consisting of protease, mannanase, and cellulase, wherein
   a. the amylase is an amylase variant of a parent amylase, wherein said amylase variant comprises
      (i) an amino acid substitution at position 25 according to the numbering of SEQ ID NO: 2,
      (ii) an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2, and
      (iii) at least 80%, preferably at least 91% identity, but less than 100% sequence identity with any of SEQ ID NO: 1,
   b. the protease a protease variant of a parent protease, wherein the protease variant comprises
      (i) an amino acid sequence comprising compared to SEQ ID NO: 16 at least two additional negative charges in the loop region of residues 98 to 104 according to the numbering of SEQ ID NO: 17, and
      (ii) an amino acid sequence which is at least 80%, but less than 100% identical to SEQ ID NO: 16,
   c. the mannanase is a mannanase variant of a parent mannanase, wherein the mannanase variant comprises
      (i) one or more amino acid substitution selected from the group consisting of 86, 89, 96, 101, 103, 107, 108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and
      (ii) an amino acid sequence which is at least 80%, but less than 100% identical to a sequence according to positions 31-490 of SEQ ID NO: 35, and
   d. the cellulase is a cellulase variant comprising an amino acid sequence which is at least 80% identical to SEQ ID NO: 42.
3. The composition according to any of the preceding embodiments, wherein the amylase variant comprises the amino acid substitution X25H and one or more amino acid substitutions selected from the group consisting of X4Q, X116K, X176K, X181T, X186E, X195F, X206Y, X225A, X251E, X320K, X405M, and X482W according to the numbering of SEQ ID NO: 2.
4. The composition according to any of the preceding embodiments, wherein the amylase comprises a deletion at one or more amino acids corresponding to positions selected from the group consisting of 181, 182, 183 and 184, preferably a deletion of two or more amino acids corresponding to positions selected from the group consisting of 181, 182, 183 and 184, preferably a deletion of amino acids corresponding to positions 181 and 182, 182 and 183, or 183 and 184, preferably of two or more, most preferably of two, amino acids corresponding to positions selected from the group consisting of R181, G182, D183, and G184, preferably D183* and G184*, wherein the numbering is according to the amino acid sequence set forth in SEQ ID NO: 2.
5. The composition according to any of the preceding embodiments, wherein the amylase variant has at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 2, preferably SEQ ID NO: 1.
6. The composition according to any of the preceding embodiments, wherein the amylase variant comprises the amino acid substitution X4G according to the numbering of SEQ ID NO: 2 and one or more, two or more, three or more, four or more, five or more, or all of the amino acid substitutions selected from the group consisting of X25H, X176K, X186E, X251E, X405M, and X482W according to the numbering of SEQ ID NO: 2, preferably comprising X4G+X176K+X186E or X4G+25H+X176K+X186E according to the numbering of SEQ ID NO: 2.
7. The composition according to any of the preceding embodiments, wherein the amylase variant comprises the amino acid substitution X25H according to the numbering of SEQ ID NO: 2 and one or more, two or more, three or more, four or more, five or more, six or more, seven or more, eight or more, or all of the amino acid substitutions selected from the group consisting of X116K, X176K, X181T, X186E, X195F, X206Y, X225A, X320K, and X482W according to the numbering of SEQ ID NO: 2, preferably comprising X25H+X176K+X186E according to the numbering of SEQ ID NO: 2.
8. The composition according to any of the preceding embodiments, wherein the amylase variant comprises the amino acid substitution X25H according to the numbering of SEQ ID NO: 2 and the amino acid substitution either X195F or X206Y according to the numbering of SEQ ID NO: 2, and one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2, preferably comprising X25H+X176K+X186E+X195F or X25H+X176K+X186E+X206Y according to the numbering of SEQ ID NO: 2.
9. The composition according to any of the preceding embodiments, wherein the amylase variant comprises the combination of amino acid substitutions (according to the numbering of SEQ ID NO: 2): X25H+X176K+X186E, X4Q+X176K+X186E, X4Q+X25H+X176K+X186E, X4Q+X25H+X176K+X186E+X251E+X405M+X482W, X25H+X176K+X186E+X195F, X25H+X176K+X186E+X206Y, X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably X4Q+X25H+X176K+X186E+X251E+X405M+X482W, and a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2).
10. The composition according to any of the preceding embodiments, wherein the amylase variant comprises or consists of the amino acid sequence set forth in SEQ ID NO: 1 with the amino acid alterations X4Q+X25H+X176K+X186E+X251E+X405M+X482W, X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably X4Q+X25H+X176K+X186E+X251E+X405M+X482W according to the numbering of SEQ ID NO: 2.
11. The composition according to any of the preceding embodiments, wherein the protease variant has at least 80%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 16 and comprises compared to SEQ ID NO: 16 the amino acid substitution X101D or X101E, preferably X101E, preferably R101E, according to the numbering of SEQ ID NO: 17.
12. The composition according to any of the preceding embodiments, wherein the protease variant comprises the amino acid substitution X101E and optionally the amino acid substitutions X3T, X4I, and X205I or the amino acid substitutions X156D and X262E according to the numbering of SEQ ID NO: 17, and an amino acid sequence which is at least 90%, but less than 100% identical to SEQ ID NO: 16.
13. The composition according to any of the preceding embodiments, wherein the protease variant comprises the amino acid substitution X101E according to the numbering of SEQ ID NO: 17, and an amino acid sequence which is at least 90%, but less than 100% identical to SEQ ID NO: 16.
14. The composition according to any of the preceding embodiments, wherein the protease variant comprises or consists of the amino acid sequence set forth in SEQ ID NO: 16 with the amino acid substitution R101E according to the numbering of SEQ ID NO: 17, i.e., only one amino acid exchange compared to SEQ ID NO: 16.
15. The composition according to any of the preceding embodiments, wherein the protease variant comprises or consists of the amino acid sequence set forth in SEQ ID NO: 16 with the amino acid substitution R101E and S3T, V41, and V205I according to the numbering of SEQ ID NO: 17.
16. The composition according to any of the preceding embodiments, wherein the protease variant comprises or consists of the amino acid sequence set forth in SEQ ID NO: 16 with the amino acid substitution R101E and S156D and L262E according to the numbering of SEQ ID NO: 17.
17. The composition according to any of the preceding embodiments, wherein the mannanase variant comprises one or more amino acid substitution selected from the group consisting of X86N, X89V/L, X96D, X101T/V, X103Y/E/A, X107N, X108G, X109Q/A, X112N, X119Y/H/T, X122S, X124E/C/D, X126E, X127A, X129M/L/F, X231Q/K/L/P/Y, X233V, X235H/R/L/Q/N/Y, X244I N/N, X254W, X255Y/H/R, X264Q/V, X270T, X272I, X273T, X274E/C/Q, X281L, X286E/L/Q/A, X289F/M/H, X290A, X296H/F/Y, X301E/C/T, X309L, X312F/Y, X314P, X317T, X319D/E, X341F, X346T, X348S/R/N/M/G, X349T/S/G/D, X352N/G, X356Y/V/T/Q/H/C, and X379V, preferably selected from X31V, X89V, X96D, X119Y/H/T, X264Q/V, X289F/M/H, X312F/Y, X348S/R/N/M/G, X349T/S/D/G, X352N/G, and X379V, according to the numbering of SEQ ID NO: 35.
18. The composition according to any of the preceding embodiments, wherein the mannanase variant comprises at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.
19. The composition according to any of the preceding embodiments, wherein the mannanase variant comprises the amino acid substitutions X89V, X96D, X119H, X264Q, X289M, X312Y, X348N, X349G, and X352G according to the numbering of SEQ ID NO: 35 and an amino acid sequence which is at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to the amino acid sequence according to positions 31-490 of SEQ ID NO: 35.
20. The composition according to any of the preceding embodiments, wherein the mannanase variant comprises or consists of the amino acid sequence according to positions 31-490 of SEQ ID NO: 35 with the amino acid substitution Q89V, N96D, A119H, E264Q, W289M, N312Y, T348N, E349G, and S352G according to the numbering of SEQ ID NO: 35.
21. The composition according to any of the preceding embodiments, wherein the cellulase variant comprises an amino acid sequence which is at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 42.
22. The composition according to any of the preceding embodiments, wherein the cellulase comprises or consists of an amino acid sequence which is 100% identical to SEQ ID NO: 42.
23. The composition according to any of the preceding embodiments, wherein the protease variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 22 and one or more amino acid substitutions selected from the group consisting of X3T, X4I, X9D/E/R, X19L, X22R/A, X43R, X62D, X63D/E, X76D, X87N, X99A/D, X101M/R/E, X103A, X1041, X116L, X118V, X128L, X129Q, X130A, X156D/E, X160D/S, X167A, X170S, X182E/D, X185E/D, X188D, X194P, X205I, X213A, X217E/D/Q, X218D, X232V, X245R, X256D/E, X259D/E, X262D/E, and X271F according to the numbering of SEQ ID NO: 17, preferably the protease variant further comprises an insertion of D and E directly adjacent C-terminally of the position 99 according to the numbering of SEQ ID NO: 17.
24. The composition according to any of the preceding embodiments, wherein the protease variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to any of SEQ ID NO: 24.
25. The composition according to any of the preceding embodiments, wherein the protease variant comprises an amino acid sequence which is at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with SEQ ID NO: 19 and one or more, preferably all substitutions selected from the group consisting of X159T, X183S, X238N, X243N, X259N and X270A, more preferably one or more, preferably all substitutions selected from the group consisting of X9T, X130D, X133A, X144K, X159T, X183S, X189T, X217M, X224A, X238N, X243N, X252T, X259N, X270A and X271E, even more preferably one or more, preferably all substitutions selected from the group consisting of X9T, X89A, X130D, X133A, X144K, X159T, X183S, X189T, X217M, X224A, X238N, X243N, X252T, X259N, X270A and X271E according to the numbering of SEQ ID NO: 19.
26. The composition according to any of the preceding embodiments, wherein the mannanase variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 40 and comprises one or more amino acid substitutions selected from the group consisting of X30T, X81P, X93Q, X95D, X118K, X138P, X139R, X200T, X202R, X210G, X260F, X283H, X2881, X294P, and X295V, preferably one or more amino acid substitutions selected from the group consisting of X260F, X2881, X294P, and X295V, according to the numbering of SEQ ID NO: 40.
27. The composition according to any of the preceding embodiments, wherein the mannanase variant comprises an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 41.
28. The composition according to any of the preceding embodiments, wherein the composition comprises an amylase and one or more, preferably two or more enzymes selected from the group consisting of protease, mannanase, and cellulase, wherein
   a. the amylase is an amylase variant comprising
      (i) the amino acid substitution X4G according to the numbering of SEQ ID NO: 2,
      (ii) one or more, two or more, three or more, four or more, five or more, or all of the amino acid substitutions selected from the group consisting of X25H, X176K, X186E, X251E, X405M, and X482W according to the numbering of SEQ ID NO: 2,
      (iii) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1, and
      (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2),
      or wherein the amylase is an amylase variant comprising
      (i) the amino acid substitution X25H according to the numbering of SEQ ID NO: 2,
      (ii) the amino acid substitution either X195F or X206Y according to the numbering of SEQ ID NO: 2,
      (iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
      (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
      (v) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1,
   b. the protease is a protease variant comprising
      (i) the amino acid substitution X101E according to the numbering of SEQ ID NO: 17, and
      (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 16,
   c. the mannanase is a mannanase variant comprising
      (i) at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35, and
      (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35, and
   d. the cellulase is a cellulase variant comprising an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 42.
29. The composition according to any of the preceding embodiments, wherein the composition comprises an amylase and one or more, preferably two or more enzymes selected from the group consisting of protease, mannanase, and cellulase, wherein
   a. the amylase variant comprises or consists of the amino acid sequence set forth in SEQ ID NO: 1 with the amino acid alterations X4Q+X25H+X176K+X186E+X251E+X405M+X482W, X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably X4Q+X25H+X176K+X186E+X251E+X405M+X482W according to the numbering of SEQ ID NO: 2,
   b. the protease is a protease variant comprising or consisting of the amino acid sequence set forth in SEQ ID NO: 16 with the amino acid substitution R101E according to the numbering of SEQ ID NO: 17, i.e., only one amino acid exchange compared to SEQ ID NO: 16,
   c. the mannanase is a mannanase variant comprising or consisting of the amino acid sequence according to positions 31-490 of SEQ ID NO: 35 with the amino acid substitution Q89V, N96D, A119H, E264Q, W289M, N312Y, T348N, E349G, and S352G according to the numbering of SEQ ID NO: 35,
   d. the cellulase is a cellulase variant comprising or consisting of an amino acid sequence which is 100% identical to SEQ ID NO: 42.
30. The composition according to any of the preceding embodiments, wherein the composition comprises an amylase and a protease, an amylase and a mannanase, or an amylase and a cellulase as described herein, preferably the composition comprises an amylase and a protease or amylase and mannanase, preferably an amylase and a mannanase as described in any of the preceding embodiments.
31. The composition according to any of the preceding embodiments, wherein the composition comprises an amylase and a protease and a mannanase as described in any of the preceding embodiments, an amylase and a protease and a cellulase as described in any of the preceding embodiments, an amylase and a mannanase and a cellulase as described in any of the preceding embodiments, preferably an amylase and a protease and a mannanase as described herein, or an amylase and a protease, a mannanase, and a cellulase as described in any of the preceding embodiments, preferably the composition comprises an amylase and a protease, a mannanase, and a cellulase as described in any of the preceding embodiments.
32. The composition according to any of the preceding embodiments, wherein the composition comprises an amylase, a protease, a mannanase, and a cellulase, wherein
   a. the amylase is an amylase variant comprising
      (i) the amino acid substitution X4G according to the numbering of SEQ ID NO: 2,
      (ii) one or more, two or more, three or more, four or more, five or more, or all of the amino acid substitutions selected from the group consisting of X25H, X176K, X186E, X251E, X405M, and X482W according to the numbering of SEQ ID NO: 2,
      (iii) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1, and
      (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2),
   or wherein the amylase is an amylase variant comprising
      (i) the amino acid substitution X25H according to the numbering of SEQ ID NO: 2,
      (ii) the amino acid substitution either X195F or X206Y according to the numbering of SEQ ID NO: 2,
      (iii) one or more, two or more, three or more, four or more, five or more, six or more, or all of the amino acid substitutions selected from the group consisting of X116K, X176K, X181T, X186E, X225A, X320K, and X482W according to the numbering of SEQ ID NO: 2,
      (iv) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 2), and
      (v) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% sequence identity with any of SEQ ID NO: 1,
   b. the protease is a protease variant comprising
      (i) the amino acid substitution X101E according to the numbering of SEQ ID NO: 17, and
      (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to SEQ ID NO: 16,
   c. the mannanase is a mannanase variant comprising
      (i) at least one, at least two, at least three, at least four, at least five, at least six, at least seven, at least eight or all amino acid substitutions X89V, X96D, X119H, X264Q, X289M, X312Y, X348N, X349G, and X352G according to the numbering of SEQ ID NO: 35, and
      (ii) an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, but less than 100% identical to a sequence according to positions 31-490 of SEQ ID NO: 35, and
   d. the cellulase is a cellulase variant comprising an amino acid sequence which is at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% identical to SEQ ID NO: 42.
33. The composition according to any of the preceding embodiments, wherein the composition comprises an amylase, a protease, a mannanase, and a cellulase, wherein
   a. the amylase variant comprises or consists of the amino acid sequence set forth in SEQ ID NO: 1 with the amino acid alterations X4Q+X25H+X176K+X186E+X251E+X405M+X482W, X25H+X116K+X176K+X181T+X186E+X195F+X225A+X320K+X482W, X25H+X116K+X176K+X181T+X186E+X206Y+X225A+X320K, or X25H+X116K+X176K+X181T+X186E+X206Y+X225A+X320K+X482W, preferably with X4Q+X25H+X176K+X186E+X251E+X405M+X482W according to the numbering of SEQ ID NO: 2,
   b. the protease is a protease variant comprising or consisting of the amino acid sequence set forth in SEQ ID NO: 16 with the amino acid substitution R101E according to the numbering of SEQ ID NO: 17, i.e., only one amino acid exchange compared to SEQ ID NO: 16,
   c. the mannanase is a mannanase variant comprising or consisting of the amino acid sequence according to positions 31-490 of SEQ ID NO: 35 with the amino acid substitution Q89V, N96D, A119H, E264Q, W289M, N312Y, T348N, E349G, and S352G according to the numbering of SEQ ID NO: 35, and
   d. the cellulase is a cellulase variant comprising or consisting of an amino acid sequence which is 100% identical to SEQ ID NO: 42.
34. The composition according to any of the preceding embodiments, wherein the composition comprises one or more additional enzyme different from the enzymes in the enzyme composition referred to in any of the preceding claims, preferably one or more additional enzyme selected from the group consisting of lipases, hemicellulases, xylanases, DNases, dispersins, pectinases, oxidoreductases, cutinases, phosphodiesterases, licheninases, xanthan-lyases, and hexosaminidases, preferably lipase.
35. The composition according to any of the preceding embodiments, wherein the combination of the amylase and the at least one enzyme selected from protease, mannanase, and cellulase in the enzyme composition provides a synergistic cleaning effect, preferably at low temperature, preferably at 30 °C or at 40 °C, preferably at 30 °C, preferably as determined in ES1-C detergent as described herein, preferably compared to an enzyme composition different to the one as described herein.
36. A formulation comprising the enzyme composition according to any one of embodiments 1 to 35 and at least one additional component, preferably a solvent.
37. The formulation according to embodiment 36, wherein the preparation comprises one or more additional enzyme different from the enzymes of the enzyme composition referred to in any of the preceding claims, preferably one or more additional enzyme selected from the group consisting of lipases, hemicellulases, xylanases, DNases, dispersins, pectinases, oxidoreductases, cutinases, phosphodiesterases, licheninases, xanthan-lyases, and hexosaminidases, preferably lipases.
38. The formulation according to embodiment 36 or 37, comprising an enzyme stabilizing system, preferably comprising a calcium salt and in case a protease is present a protease inhibitor.
39. The formulation according to any of embodiment 36 to 38, wherein the enzyme formulation comprises a protease inhibitor in case a protease is present in the enzyme preparation, preferably wherein the protease inhibitor is a phenyboronic acid derivative (preferably 4-FPBA) or a peptide aldehyde (preferably Z-GAY-H or Z-VAL-H).
40. Method of making a composition or formulation according to any of the preceding embodiments comprising the step of mixing the amylase with at least one enzyme selected from protease, mannanase, and cellulase.
41. A detergent composition comprising the amylase variant according to any one of embodiments 1 to 35, preferably a laundry detergent composition or a hard surface cleaning detergent composition, most preferably laundry detergent composition.
42. The detergent composition according to embodiment 41, wherein the detergent composition comprises a component selected from the group consisting of an additional enzyme, enzyme stabilizing system, surfactant, defoamer, builder, polymer, bleaching system (bleach), rheology modifier, hydrotrope, softening agent, desiccant, whitening agent, buffer, preservative, anti-corrosion additive, dyestuff and fragrance.
43. The detergent composition according to embodiment 41 or 42, wherein the detergent composition comprises one or more additional enzyme different from the enzymes in the enzyme composition referred to in any of the preceding claims, preferably one or more additional enzyme selected from the group consisting of lipases, hemicellulases, xylanases, DNases, dispersins, pectinases, oxidoreductases, cutinases, phosphodiesterases, licheninases, xanthan-lyases, and hexosaminidases, preferably lipases.
44. The detergent composition according to any of embodiment 41 to 43, comprising a builder, wherein the builder is selected from MDGA, GLDA, DTPMP, HEDP, and EDDS, preferably MDGA or EDDS.
45. The detergent composition according to embodiment 44, wherein the builder is a non-phosphate based builder.
46. The detergent composition according to any of embodiment 41 to 45, comprising a surfactant, wherein the surfactant is selected from non-ionic, anionic, cationic, amphoteric, and combinations thereof.
47. The detergent composition according to any of embodiment 41 to 46, wherein the detergent composition is devoid of anionic surfactants.
48. The detergent composition according to any of embodiment 41 to 47, wherein the surfactant and/or the builder is bio-degradable and/or bio-based.
49. The detergent composition according to any of embodiment 41 to 48, wherein the detergent composition is liquid or solid.
50. The detergent composition according to any of embodiment 41 to 49, wherein the detergent composition is in the form of a pouch.
51. The detergent composition according to any of embodiment 41 to 50, wherein the detergent composition is a liquid laundry detergent composition.
52. The detergent composition according to any of embodiment 41 to 51, wherein the detergent composition is boron-free.
53. The detergent composition according to any of embodiment 41 to 52, wherein the detergent composition does not comprise a preservative.
54. Method for cleaning comprising the step of contacting a textile or a hard surface, preferably textile, with a composition according to any of the preceding embodiments 1-35, preferably at low temperature, preferably at 30 °C or at 40 °C, preferably at 30 °C.
55. The method according to embodiment 54, wherein the method for cleaning is for cleaning a complex stain from a textile or a hard surface.
56. Use of a composition according to any of the preceding embodiments 1-35 for providing a synergistic cleaning effect, preferably on complex stains, preferably at low temperature, preferably at 30 °C or at 40 °C, preferably at 30 °C, preferably as determined in ES1-C detergent as described herein, preferably compared to an enzyme composition different to the one as described herein.
57. Use of a composition according to any of the preceding embodiments 1-35 for removal of a complex stain, preferably at low temperature, preferably at 30 °C or at 40 °C, preferably at 30 °C.
58. Use of a composition according to any of the preceding embodiments 1-35 for providing an improved wash performance, preferably on complex stains, preferably at low temperature, preferably at 30 °C or at 40 °C, preferably at 30 °C, preferably as determined in ES1-C detergent as described herein, preferably compared to an enzyme composition different to the one as described herein.

### Examples

### Example 1: Synergies in Wash Performance when Using Enzyme Blends

### Application Tests

Several soiled swatches were washed together with cotton ballast fabric and 20 steel balls at 30 °C in a liquid laundry formulation with different selected single enzymes (amylase, protease, cellulase, and mannanase) and combinations of said enzymes (i.e., enzyme blends). Each stain was washed in duplicates. After the wash, the fabrics were rinsed, spin-, and air-dried. The washing performance was determined by measuring the CIELab values of the soiled fabrics before and after wash using the MACH5 multi area color measurement. For data evaluation mean values were determined, ΔE was calculated between unwashed and washed stains and ΔΔE was calculated between detergent with enzymes and detergent without enzymes. Synergies of using blends vs. using single enzymes were determined by calculating the difference between the wash performance of an enzyme blend and the wash performance of the added single enzyme performances relevant for the specific blend.

TABLE 1
Washing conditions

| | |
|---|---|
| Test equipment | Launder-o-meter, LP2 Typ, |
| | SDL Atlas Inc., USA |
| Washing liquor | 250 ml |
| Washing time and temperature | 20 min at 30 °C |
| Detergent dosage | 3 g/L |
| Fabric/liquor ratio | 1:14 |
| Washing cycles | 1 |
| Water hardness | 2.5 mmol/l Ca²⁺:Mg²⁺:HCO₃⁻ 4:1:8 |
| Ballast fabric | 10 g cotton fabric |
| Sum ballast + soiled fabric | 17.5 g |
| Soiled fabric | 2 pieces 5 × 5 cm CFT-CS 06 ¹⁾ |
| | 2 pieces 5 × 5 cm EMPA 163 ²⁾ |
| | 2 pieces 5 × 5 cm EMPA 165 ³⁾ |
| Laundry formulation | ES1C formulation: |
| | Maranil DBS/LC: 5.5 w/w % |
| | Edenor coco fatty acid K12-18: 2.4 w/w % |
| | Texapon N70: 5.4 w/w % |
| | NaOH: 2.2 w/w % |
| | Lutensol AO 7: 5.4 w/w % |
| | 1.2 Propylene glycol: 6 w/w % |
| | Ethanol: 2 w/w % |
| | Sodium citrate: 3 w/w % |
| | Water up to 90% |
| Enzyme concentrations | Protease: 1.13 ppm per wash |
| | Amylase: 0.3 ppm per wash |
| | Mannanase: 0.03 ppm per wash |
| | Cellulase: 0.15 ppm per wash |

¹⁾ CFT-CS 06, Producer: Center for Testmaterials BV, NL-3130 AC Vlaardingen
²⁾ EMPA 163, Producer: Swissatest Testmaterialien AG, Mövenstrasse 12, 9015 St. Gallen, Schweiz
³⁾ EMPA 165, Producer: Swissatest Testmaterialien AG, Mövenstrasse 12, 9015 St. Gallen, Schweiz

TABLE 2
Synergistic effects when using enzyme blends including Amylase A

| | | | | Synergy | Synergy | Synergy |
|---|---|---|---|---|---|---|
| | | | | CFT-CS | EMPA | EMPA |
| Amylase* | Protease* | Mannanase* | Cellulase* | 06 | 163 | 165 |
| Amylase A | Protease A | — | — | — | 2.2 | 1.6 |
| Amylase A | — | Mannanase A | — | 1.6 | — | 3.2 |
| Amylase A | — | — | Cellulase A | — | 2.4 | 2.2 |
| Amylase A | Protease A | Mannanase A | — | 2.5 | 3.8 | 4.4 |
| Amylase A | Protease A | — | Cellulase A | — | 3.7 | 1.9 |
| Amylase A | — | Mannanase A | Cellulase A | 1.2 | 3.0 | 2.9 |
| Amylase A | Protease A | Mannanase A | Cellulase A | 1.8 | 5.1 | 4.9 |
| Amylase A | Protease B | Mannanase A | Cellulase A | 0.7 | 2.2 | 1.5 |
| Amylase A | Protease A | Mannanase B | — | — | 1.9 | 0.5 |
| Amylase A | Protease B | Mannanase B | — | — | 1.0 | — |
| Amylase A | Protease B | — | Cellulase A | — | 1.3 | — |

*Amylase A = SEQ ID NO: 1 with N25H and in addition G4Q, R176K, G186E, T251E, L405M, Y482W, Protease A = SEQ ID NO: 16 with R101E (numbering of SEQ ID NO: 17), Mannanase A = amino acids 31-490 if SEQ ID NO: 35 with Q89V, N96D, A119H, E264Q, W289M, N312Y, T348N, E349G, and S352G (numbering of SEQ ID NO: 35), Cellulase A = SEQ ID NO: 42, Protease B = SEQ ID NO: 24, Mannanase B = SEQ ID NO: 41

As can be derived from Table 2, using enzyme blends including Amylase A provides a synergistic cleaning effect.

TABLE 3
Synergistic effects when using enzyme blends including Amylase A
in comparison to blends including Amylase B (reference amylase)

| | | | | Synergy | Synergy | Synergy |
|---|---|---|---|---|---|---|
| | | | | CFT-CS | EMPA | EMPA |
| Amylase* | Protease* | Mannanase* | Cellulase* | 06 | 163 | 165 |
| Amylase A | Protease A | Mannanase A | — | 2.5 | 3.8 | 4.4 |
| Amylase B | Protease A | Mannanase A | — | — | 1.4 | — |
| Amylase A | Protease A | — | Cellulase A | — | 3.7 | 1.9 |
| Amylase B | Protease A | — | Cellulase A | — | 1.9 | — |
| Amylase A | Protease B | — | Cellulase A | — | 1.3 | — |
| Amylase B | Protease B | — | Cellulase A | — | 1.1 | 0.5 |
| Amylase A | Protease A | Mannanase A | Cellulase A | 1.8 | 5.1 | 4.9 |
| Amylase B | Protease A | Mannanase A | Cellulase A | 1.8 | 0.5 | 4.4 |

*Amylase A = SEQ ID NO: 1 with N25H and in addition G4Q, R176K, G186E, T251E, L405M, Y482W (numbering of SEQ ID NO: 2), Protease A = SEQ ID NO: 16 with R101E (numbering of SEQ ID NO: 17), Mannanase A = amino acids 31-490 if SEQ ID NO: 35, Cellulase A = SEQ ID NO: 42, Amylase B (reference amylase) = SEQ ID NO: 6, Protease B = SEQ ID NO: 24

As can be derived from Table 3, using enzyme blends including Amylase A provides an improved synergistic cleaning effect compared to using the reference Amylase B.

1. A composition comprising an amylase and one or more enzymes selected from the group consisting of protease, mannanase, and cellulase, wherein
a. the amylase is an amylase variant of a parent amylase, wherein said amylase variant
(i) comprises an amino acid substitution at position 25 according to the numbering of SEQ ID NO: 2,
(ii) comprises an amino acid substitution at one or more amino acid positions corresponding to amino acid positions selected from the group consisting of 4, 116, 176, 181, 186, 195, 206, 225, 251, 320, 405, and 482 according to the numbering of SEQ ID NO: 2, and
(iii) has at least 60%, but less than 100% sequence identity with any of SEQ ID NO: 1-15,
b. the protease is selected from
I. a protease variant of a parent protease, wherein the protease variant comprises
(i) an amino acid sequence comprising compared to SEQ ID NO: 16 at least two additional negative charges in the loop region of residues 98 to 104 according to the numbering of SEQ ID NO: 17, and
(ii) an amino acid sequence which is at least 60%, but less than 100% identical to any of SEQ ID NO: 16-34, or
II. a protease variant of a parent protease, wherein the protease variant comprises
(i) an amino acid substitution at one or more amino acid positions corresponding to amino acid positions selected from the group consisting of 3, 4, 9, 19, 22, 43, 62, 63, 76, 87, 99, 101, 103, 104, 116, 118, 128, 129, 130, 156, 160, 167, 170, 182, 185, 188, 194, 205, 213, 217, 218, 232, 245, 256, 259, 262, and 271 according to the numbering of SEQ ID NO: 17 and an amino acid sequence which has at least 60%, but less than 100% sequence identity with SEQ ID NO: 22, or
(ii) an amino acid substitution at one or more amino acid positions corresponding to amino acid positions selected from the group consisting of 24, 53, 76, 78, 101, 128, and 217 according to the numbering of SEQ ID NO: 17 and an amino acid sequence which has at least 60%, but less than 100% sequence identity with SEQ ID NO: 17, or
(iii) an amino acid substitution at one or more amino acid positions corresponding to amino acid positions selected from the group consisting of 9, 89, 130, 133, 144, 159, 183, 189, 217, 224, 238, 243, 252, 259, 270 and 271 according to the numbering of SEQ ID NO: 19 and an amino acid sequence which has at least 60%, but less than 100% sequence identity with SEQ ID NO: 19, or
(iv) an amino acid sequence which has at least 60% sequence identity with SEQ ID NO: 22, SEQ ID NO: 21, SEQ ID NO: 30, SEQ ID NO: 20, SEQ ID NO: 25, SEQ ID NO: 18, SEQ ID NO: 19, or SEQ ID NO: 31,
c. the mannanase is selected from
I. a mannanase variant of a parent mannanase, wherein the mannanase variant comprises
(i) one or more amino acid substitution selected from the group consisting of 86, 89, 96, 101, 103, 107,108, 109, 112, 119, 122, 124, 126, 127, 129, 231, 233, 235, 244, 254, 255, 264, 270, 272, 273, 274, 281, 286, 289, 290, 296, 301, 309, 312, 314, 317, 319, 341, 346, 348, 349, 352, 356, and 379 according to the numbering of SEQ ID NO: 35, and
(ii) an amino acid sequence which is at least 60%, but less than 100% identical to a sequence according to positions 31-490 of SEQ ID NO: 35, or
II. a mannanase variant of a parent mannanase, wherein the mannanase variant comprises
(i) an amino acid substitution at one or more amino acid positions corresponding to amino acid positions selected from the group consisting of 24, 123, and 256 according to the numbering of SEQ ID NO: 39 and an amino acid sequence which is at least 60%, but less than 100% identical to SEQ ID NO: 39, or
(ii) an amino acid substitution at one or more amino acid positions corresponding to amino acid positions selected from the group consisting of 10, 19, 30, 38, 59, 60, 67, 97, 103, 129, 143, 167, 168, 184, 225, 228, 235, 244, 258, 261, and 298 according to the numbering of SEQ ID NO: 38 and an amino acid sequence which is at least 60%, but less than 100% identical to SEQ ID NO: 38, or
(iii) an amino acid sequence which is at least 60% identical to SEQ ID NO: 40, or
(iv) an amino acid substitution at one or more amino acid positions corresponding to amino acid positions selected from the group consisting of 30, 81, 93, 95, 118, 138, 139, 200, 202, 210, 260, 283, 288, 294, and 295 according to the numbering of SEQ ID NO: 40 and an amino acid sequence which is at least 60%, but less than 100% identical to SEQ ID NO: 40,
d. the cellulase is selected from
I. a cellulase variant comprising an amino acid sequence which is at least 60% identical to SEQ ID NO: 42, or
II. a cellulase variant comprising an amino acid sequence which is at least 60% identical to SEQ ID NO: 46, SEQ ID NO: 43, SEQ ID NO: 45, SEQ ID NO: 44, or SEQ ID NO: 47.

2. The composition according to claim 1, wherein the amylase variant has at least 80%, but less than 100% sequence identity with SEQ ID NO: 1 and comprises the amino acid substitution X25H and one or more amino acid substitutions selected from the group consisting of X4Q, X116K, X176K, X181T, X186E, X195F, X206Y, X225A, X251E, X320K, X405M, and X482W according to the numbering of SEQ ID NO: 2.

3. The composition according to claim 1, wherein the amylase comprises a deletion at one or more amino acids corresponding to positions selected from the group consisting of 181, 182, 183 and 184, optionally a deletion of two or more amino acids corresponding to positions selected from the group consisting of 181, 182, 183 and 184, optionally a deletion of amino acids corresponding to positions 181 and 182, 182 and 183, or 183 and 184, wherein the numbering is according to the amino acid sequence set forth in SEQ ID NO: 2.

4. The composition according to claim 1, wherein the protease variant has at least 80%, but less than 100% sequence identity with SEQ ID NO: 16 and comprises an amino acid substitution at position 101, according to the numbering of SEQ ID NO: 17.

5. The composition according to claim 1, wherein the mannanase variant has at least 80%, but less than 100% sequence identity with SEQ ID NO: 35 and comprises one or more amino acid substitution selected from the group consisting of X86N, X89V/L, X96D, X101T/V, X103Y/E/A, X107N, X108G, X109Q/A, X112N, X119Y/H/T, X122S, X124E/C/D, X126E, X127A, X129M/L/F, X231Q/K/L/P/Y, X233V, X235H/R/L/Q/N/Y, X244I/V/N, X254W, X255Y/H/R, X264Q/V, X270T, X272I, X273T, X274E/C/Q, X281L, X286E/L/Q/A, X289F/M/H, X290A, X296H/F/Y, X301E/C/T, X309L, X312F/Y, X314P, X317T, X319D/E, X341F, X346T, X348S/R/N/M/G, X349T/S/G/D, X352N/G, X356Y/V/T/Q/H/C, and X379V, according to the numbering of SEQ ID NO: 35.

6. The composition according to claim 1, wherein the cellulase variant comprises an amino acid sequence which is at least 65%, at least 70%, at least 75%, at least 80%, at least 85%, at least 90%, at least 95%, at least 98% or 100% identical to SEQ ID NO: 42.

7. The composition according to claim 1, wherein the composition comprises one or more additional enzymes selected from the group consisting of lipases, hemicellulases, xylanases, DNases, dispersins, pectinases, oxidoreductases, cutinases, phosphodiesterases, licheninases, xanthan-lyases, and hexosaminidases.

8. The composition according to claim 1, wherein the composition is a detergent composition, optionally a laundry detergent composition or a hard surface cleaning detergent composition.

9. The composition according to claim 8, wherein the detergent composition comprises a detergent component selected from the group consisting of enzyme stabilizing system, surfactant, defoamer, builder, polymer, bleaching system (bleach), rheology modifier, hydrotrope, softening agent, desiccant, whitening agent, buffer, suds suppressors, preservative, anti-corrosion additive, dyestuff and fragrance.

10. The composition according to claim 1, wherein the combination of the amylase and the at least one enzyme selected from protease, mannanase, and cellulase in the enzyme composition provides a synergistic cleaning effect.

11. A method of making a composition according to claim 1 comprising the step of mixing the amylase with at least one enzyme selected from protease, mannanase, and cellulase.

12. A method for cleaning comprising the step of contacting a textile or a hard surface with a composition according to claim 1, optionally at low temperature.

13. The method according to claim 12, wherein the method for cleaning is for cleaning a complex stain from a textile or a hard surface.

14. A method for providing a synergistic cleaning effect, optionally at low temperature comprising applying the composition according to claim 1 to a surface.

15. A method for removal of a complex stain, optionally at low temperature comprising applying the composition according to claim 1 to a complex stain.

16. The composition according to claim 1, wherein the composition comprises amylase and two or more enzymes selected from the group consisting of protease, mannanase, and cellulase.
